(19) United States
(12) Patent Application Publication
(10) Pub. No.: US 2023/0247997 A1
(43) Pub. Date: Aug. 10, 2023

(54) NEMATICIDE COMPOUNDS, COMPOSITIONS, AND METHODS OF THEIR MAKING AND USE

(71) Applicant: IOWA STATE UNIVERSITY RESEARCH FOUNDATION, INC., Ames, IA (US)

(72) Inventors: James KLIMAVICZ, Ames, IA (US); Joel COATS, Ames, IA (US)

(21) Appl. No.: 18/004,687

(22) PCT Filed: Jul. 14, 2021
(86) PCT No.: PCT/US2021/041582

§ 371 Date: Jan. 9, 2023

Related U.S. Application Data
(60) Provisional application No. 63/051,735, filed on Jul. 14, 2020.
(60) Provisional application No. 63/051,692, filed on Jul. 14, 2020.

(51) Int. Cl. A01N 43/82; C07D 285/08; C07D 251/22; A01N 43/66; A01P 5/00

(52) U.S. Cl. CPC A01N 43/82; C07D 285/08; C07D 251/22; A01N 43/66; A01P 5/00

(57) ABSTRACT

The present application relates to compounds of formulae (I)-(VII) as defined herein, compositions containing these compounds, methods of their use, and methods of making. The compounds have nematacide activity.

This application claims the priority benefit of U.S. Provisional Patent Application Ser. No. 63/051,692 filed Jul. 14, 2020 and U.S. Provisional Patent Application Ser. No. 63/051,735, filed Jul. 14, 2020, which are hereby incorporated by reference in their entirety.

### FIELD

The present application relates to compounds, compositions containing the compounds, methods of their use, and methods of making compounds.

### BACKGROUND

1,2,4-Thiadiazoles

Over the past decade, 1,2,4-thiadiazoles have gained significant attention as potential building blocks for the development of new biologically active compounds (Frija et al., “Building 1,2,4-thiadiazole: Ten Years of Progress,” Eur. J. Org. Chem. 2017:2670-2682 (2017)). FIG. 1 shows several 1,2,4-thiadiazoles, including cefozopran, a cephalosporin antibiotic with improved activity against methicillin-resistant Staphylococcus aureus (Miyake et al., “Studies on Condensed-heterocyclic Azolium Cephalosporins,” J. Antibiot. 45:709-720 (1992)), an inhibitor of the protease cathepsin B (Leung-Toung et al., “1,2,4-Thiadiazole: A Novel Cathepsin B Inhibitor,” Biorg. Med. Chem. 11:5529-5537 (2003)), and the agriculturally important fungicide etridiazole (Radzuhn & Lyr, “On the Mode of Action of the Fungicide Etridiazole,” Pestic. Biochem. Physiol. 22:14-23 (1984); Rochester et al., “Etridiazole May Conserve Applied Nitrogen and Increase Yield of Irrigated Cotton,” Soil Res. 32:1287-1300 (1994)). The first natural product containing the 1,2,4-thiadiazole structure, dendroine (FIG. 1) was isolated in 1980 (Heitz et al., “Nouveau Derive Indolique du Thiadiazole-1,2,4, Isole d'un Tunicier (Dendrodoa grossularia),” Tetrahedron Lett. 21:1457-1458 (1980)). Since this time, several other natural 1,2,4-thiadiazole derivatives have been identified (Chen et al., “Antiviral Stereoisomers of 3,5-bis(2-hydroxybut-3-en-1-yl)-1,2,4-Thiadiazole From the Roots of Isatis indigotica,” Chin. Chem. Lett. 27:643-648 (2016); Pham et al., “New Cytotoxic 1,2,4-thiadiazole Alkaloids From the Ascidian Polycarpa aurata,” Org. Lett. 15:2230-2233 (2013)). Recently, π-conjugated molecules containing a 1,2,4-thiadiazole core have been reported for use in phosphorescent organic light-emitting diodes (Jin et al., “Construction of High Tg Bipolar Host Materials With Balanced Electron-hole Mobility Based on 1,2,4-thiadiazole for Phosphorescent Organic Light-emitting Diodes,” Chem. Mater. 26:2388-2395 (2014)). Additionally, peptide mimics containing 1,2,4-thiadiazole moieties have been proposed as chelating compounds for Cu^{II} ions (Xie et al., “Contemplating 1,2,4-thiadiazole-inspired Cyclic Peptide Mimics: A Computational Investigation,” Aust. J. Chem. 72:894-899 (2019)).

As a bioisostere of carboxylic acid esters (Lassalas et al., “Structure Property Relationships of Carboxylic Acid Isosteres,” J. Med. Chem. 59:3183-3203 (2016)), the 1,2,4-thiadiazole ring remains an attractive target for the development of new pharmaceutical compounds due to their biological activity. 5-Alkylthio-1,2,4-thiadiazoles (“ATTD”s) have also be used as versatile intermediates in the synthesis of other 1,2,4-thiadiazoles (Park et al., “Parallel Synthesis of Drug-like 5-amino-substituted 1,2,4-thiadiazole Libraries Using Cyclization Reactions of a Carboxamidine Dithiocarbamate Linker,” Synthesis 2009:913-920 (2009); Baumann & Baxendale, “A Continuous Flow Synthesis and Derivatization of 1,2,4-thiadiazoles,” Biorg. Med. Chem. 25:6218-6223 (2017)). While synthetic methods for producing 1,2,4-thiadiazoles have been comprehensively documented in several reviews (Frija et al., “Building 1,2,4-thiadiazole: Ten Years of Progress,” Eur. J. Org. Chem. 2017:2670-2682 (2017); Franz & Dhingra, “4.25-1,2,4-Thiadiazoles,” In Comprehensive Heterocyclic Chemistry, Katritzky, A. R.; Rees, C. W., Eds. Pergamon: Oxford, pp 463-511 (1984); Kurzer, F., “1,2,4-Thiadiazoles,” Adv. Heterocycl. Chem. 5:119-204 (1965); Wilkins & Bradley, 4.08—“1,2,4-Thiadiazoles,” In Comprehensive Heterocyclic Chemistry II, Katritzky, A. R.; Rees, C. W.; Scriven, E. F. V., Eds. Pergamon: Oxford, pp 307-354 (1996); Wilkins, D. J., 5.08—“1,2,4-Thiadiazoles,” In Comprehensive Heterocyclic Chemistry III, Katritzky, A. R.; Ramsden, C. A.; Scriven, E. F. V.; Taylor, R. J. K., Eds. Elsevier: Oxford, pp 487-513 (2008)), the chemical space surrounding 1,2,4-thiadiazoles remains underexplored compared to other thiadiazoles and oxadiazoles. Methods for producing ATTDs are particularly uncommon in the literature. Dipotassium cyanodithioiminocarbonate can be oxidized with chlorine gas to produce 3-chloro-1,2,4-thiadiazol-5-ylsulfenyl chloride (Thaler & McDivitt, “Synthesis and Some Reactions of 1,2,4-thiadiazolylsulfenyl Chlorides,” J. Org. Chem. 36:14-18 (1971)). Alternatively, refluxing amidines or amidoximes with carbon disulfide, elemental sulfur, and sodium methoxide yields the corresponding 1,2,4-thiadiazole-5-thione (U.S. Pat. No. 3,478,045 to Hahn & Seefelder; Tiemann, F., “Ueber Schwefelhaltige Umwandlungs-producte der Amidoxime,” Ber. Dtsch. Chem. Ges. 24:369-377 (1891)), which may then be deprotonated and S-alkylated (FIG. 2, a)) (Agirbag et al., “Synthesis and Methylation of Some 1,2,4-thiadiazole-5-thiones,” Phosphorus, Sulfur, Silicon Relat. Elem. 66:321-324 (1992)). Amidines can also be reacted with trichloromethylsulfenyl chloride, producing 5-chloro-1,2,4-thiadiazoles in modest yield, which react with thiourea to give 1,2,4-thiadiazole-5-thiones, or with thiolates to give the 5-thiolated-1,2,4-thiadiazole (FIG. 2, b)) (Goerdeler et al., “Über 1.2.4-Thiodiazole, VIII. Synthese von 5-Chlor-1.2.4-Thiodiazolen aus Perchlormethylmercaptan und Amidinen,” Chem. Ber. 90:182-187 (1957); Noguchi et al., “Studies on the Selective Toxicity. VIII.: Biological activities of 1, 2, 4-thiadiazole Derivatives,” Yakugaku Zasshi 88:1437-1449 (1968)).

It appears that Park et al., “Parallel Synthesis of Drug-like 5-amino-substituted 1,2,4-thiadiazole Libraries Using Cyclization Reactions of a Carboxamidine Dithiocarbamate Linker,” Synthesis 2009:913-920 (2009) have published the only synthesis of an ATTD from an amidine, carbon disulfide, and an alkyl halide that does not use a 1,2,4-thiadiazole-5-thione intermediate. In this case, S-benzyl N-(iminobenzyl)dithiocarbamate was synthesized from the benzamidine hydrochloride hydrate in the presence of benzyl chloride, three equivalents of cesium carbonate, three equivalents of carbon disulfide, and catalytic tetrabutylammonium iodide (TBAI) in tetrahydrofuran (THF) for 24 hours. The dithiocarbamate was then isolated and purified in 74% yield, and 5-benzylthio-3-phenyl-1,2,4-thiadiazole was produced in 99% yield after 24 hours at 60° C. with excess tosyl chloride and pyridine in dichloroethane (FIG. 2, c)) (Park et al., “Parallel Synthesis of Drug-like 5-amino-substituted 1,2,4-thiadiazole Libraries Using Cyclization Reactions of a Carboxamidine Dithiocarbamate Linker,” Synthesis 2009:913-920 (2009)).

Ketene Dithioacetals

Ketene dithioacetals are irrefutably valuable building blocks in organic synthesis (Kolb, M., “Ketene Dithioacetals in Organic Synthesis: Recent Developments,” Synthesis 1990:171-190 (1990); Metwally & Abdel-Latif, “Versatile α-Oxoketene Dithioacetals and Analogues in Heterocycle Synthesis,” Journal of Sulfur Chemistry 25:359-379 (2004); Pan et al., “Recent Developments of Ketene Dithioacetal Chemistry,” Chem. Soc. Rev. 42:1251-1286 (2013); Huang et al., “Ketene Dithioacetals in Organic Synthesis,” Tetrahedron Lett. 61:151363 (2020)), serving as intermediates for the synthesis of various derivatized benzenes (Wang et al., “Tandem [4+1+1] Annulation and Metal-free Aerobic Oxidative Aromatization: Straightforward Synthesis of Highly Substituted Phenols From One Aldehyde and Two Ketones,” Chem. Commun. 46:9061-9063 (2010); Mellor et al., “Reactions of Ketene Dithioacetals With Bis-nucleophiles: Synthesis of Novel Heterocyclic Thiols,” Tetrahedron 53:17163-17170 (1997)), azoles (Sharma et al., “Efficient Trifluoromethylation of C(sp2)-H Functionalized α-Oxoketene Dithioacetals: A Route to the Regioselective Synthesis of Functionalized Trifluoromethylated Pyrazoles,” RSC Advances 7:10150-10153 (2017); Shahcheragh et al., “Straightforward Synthesis of Novel Substituted 1,3,4-thiadiazole Derivatives in Choline Chloride-based Deep Eutectic Solvent,” Tetrahedron Lett. 58:855-859 (2017); Wu et al., “Regiodivergent Heterocyclization: A Strategy for the Synthesis of Substituted Pyrroles and Furans Using α-Formyl Ketene Dithioacetals as Common Precursors,” Chem. Commun. 50:1797-1800 (2014); Jedinák et al., “The Synthesis and Biological Evaluation of N-Substituted 1H-Benzimidazol-2-yl-1H-pyrazole-3,5-diamines,” J. Heterocycl. Chem. 53:499-507 (2016); Dieter & Chang, “Synthesis of Isoxazoles and Isothiazoles From α-oxo ketene Dithioacetals,” J. Org. Chem. 54:1088-1092 (1989)), azines (Kumar et al., “A Simple and General Approach for the Synthesis of Highly Functionalized 6-oxo-1,6-dihydropyridines,” Tetrahedron 69:5112-5118 (2013); Yang et al., “A Facile and Efficient Synthesis of Fully Substituted Pyridin-2(1H)-ones From α-Oxoketene-S,S-acetals,” Tetrahedron 67:8343-8347 (2011); Verma et al., “Heteroaromatic Annulation Studies on 2-[bis(methylthio)methylene]-1,3-indanedione: Efficient Routes to Indenofused Heterocycles,” Tetrahedron 66:7389-7398 (2010); Liu et al., “Facile Synthesis of 3-aryl-5-cyano-6-methylthio-pyrimidine-2,4-diones,” Synth. Commun. 29:3143-3147 (1999)), pyrones (Kumar et al., “Highly Convenient Regioselective Synthesis of Functionalized Arylated Benzene From Ketene-S,S-acetal Under Mild Conditions at Room Temperature,” Tetrahedron Lett. 50:680-683 (2009)), and other heterocycles (Zheng et al., “Iodine-catalyzed Intramolecular Oxidative Thiolation of Vinylic Carbon-Hydrogen Bonds Via Tandem Iodocyclization and Dehydroiodination: Construction of 2-methylene-3-thiophenones,” Adv. Synth. Catal. 356:743-748 (2014); Okazaki et al., “Reactions of α-oxo Ketene Dithioacetals With Dimethylsulfonium Methylide: A New Versatile Synthesis of Furans and Butenolides,” J. Org. Chem. 49:3819-3824 (1984)); as masked carbonyl moieties or carbonyl homologation intermediates (Suzuki et al., “Stereocontrolled Asymmetric Total Synthesis of Protomycinolide IV,” Journal of the American Chemical Society 108:5221-5229 (1986); Saito et al., “Total Synthesis of the Furaquinocins,” Journal of the American Chemical Society 120:11633-11644 (1998); Hanessian et al., “Substrate-Controlled and Organocatalytic Asymmetric Synthesis of Carbocyclic Amino Acid Dipeptide Mimetics,” J. Org. Chem. 75:2861-2876 (2010); Myrboh et al., Polarized Ketene Dithioacetals, “A New General Highly Stereoselective and Regiospecific Method for Homologation of Ketones to α,β-Unsaturated Esters Via α-Oxoketene Dithioacetals.” J. Org. Chem. 48:5327-5332 (1983); Carey & Neergaard, “Reactions of Ketene Thioacetals With Electrophiles. Homologation of Aldehydes,” J. Org. Chem. 36:2731-2735 (1971); Shingate et al., “Ionic Hydrogenation of C-20, 22-ketene Dithioacetal: Stereoselective Synthesis of Steroidal C (20R) Adehydes,” Chem. Commun. 2194-2195 (2004)), or as nucleophilic building blocks (Yuan et al., “Copper(II)-catalyzed C—C Bond-forming Reactions of α-electron-Withdrawing Group-substituted Ketene S,S-acetals With Carbonyl Compounds and a Facile Synthesis of Coumarins,” Adv. Synth. Catal. 351:112-116 (2009); Yuan et al., “Unexpected Hydrobromic Acid-catalyzed C—C Bond-forming Reactions and Facile Synthesis of Coumarins and Benzofurans Based on Ketene Dithioacetals,” Chem. Eur. J. 16:13450-13457 (2010)). Ketene dithioacetals possessing an electron-withdrawing group (EWG)-including esters, nitriles, ketones, phosphonates, sulfoxides, or nitro groups—on the α-carbon are frequently encountered due to their ease of synthesis from the base-mediated addition of carbon disulfide to an activated carbon, followed by the addition of an alkyl halide (Freund, E., “Uber die Einwirkung von Schwefelkohlenstoff auf Nitro-methan,” Ber. Dtsch. Chem. Ges. 52:542-544 (1919)). Despite their utility, ketene dithioacetals without an α-EWG (“KDTA”s) are less prevalent. KDTAs have been used as reagents in a variety of cycloadditions, including inverse electron demand Diels-Alder reactions with substrates including pyrones (Bates et al., “Diels-Alder Reactions of 1,1-IPDR Bis(methylthio)ethene with Pyran-2-ones,” Aust. J. Chem. 51:383-388 (1998)), tropone (Dahnke & Paquette, “Inverse Electron-Demand Diels-Alder Cycloaddition of a Ketene Dithioacetal. Copper Hydride-Promoted Reduction of a Conjugated Enone. 9-Dithiolanobicyclo[3.2.2]Non-6-En-2-One from Tropone,” Org. Synth. 71:181 (1993)), and α,β-unsaturated carbonyls (Denmark & Sternberg, “Intramolecular [4+2] Cycloadditions of (Z)-α,β-unsaturated Aldehydes with Vinyl Sulfides and Ketene Dithioacetals,” Journal of the American Chemical Society 108:8277-8279 (1986)), the aza-Diels-Alder reaction (Cheng et al., “Ketene Dithioacetals in the Aza-Diels-Alder Reaction with N-Arylimines: A Versatile Approach to Tetrahydroquinolines, 2,3-Dihydro-4-quinolones, and 4-Quinolones,” Org. Lett. 4:4411-4414 (2002)), titanium-catalyzed [2+2] cycloadditions (Narasaka et al., “Asymmetric [2+2] Cycloaddition Reaction Catalyzed by a Chiral Titanium Reagent,” Journal of the American Chemical Society 114:8869-8885 (1992)), and [3+2] dipolar additions with nitrile oxides and nitrones (Yamamoto et al., “1,3-Dipolar Cycloaddition of N-oxide With 2-methylene-1,3-dithiane,” Heterocycles 26:755-758 (1987)). Additionally, KDTAs have served as precursors to electron-deficient ketene dithioacetal S,S′-dioxides (Wedel & Podlech, “Alkylidene[1,3]dithiolane-1,3-dioxides as Potent Michael-type Acceptors,” Synlett 2006:2043-2046 (2006)), difluorooxymethylene-bridge compounds via oxidative alkoxydifluorodesulfuration (Kirsch et al., “Difluorooxymethylene-Bridged Liquid Crystals: A Novel Synthesis Based on the Oxidative Alkoxydifluorodesulfuration of Dithianylium Salts,” Angew. Chem. Int. Ed. 40:1480-1484 (2001)), and amines and cyclic amino acids from the addition to azides (Moss et al., “Ketene-S,S-acetals as 1,3-dipolarophiles Towards Azides. A New Synthetic Entry Into Cyclic Amino Acids,” Tetrahedron 48:7551-7564 (1992)).

Methods for the synthesis of KDTAs are somewhat limited. The most common method is the Horner-Wadsworth-Emmons (“HWE”) reaction using dialkyl bis(alkylthio)methylphosphonates (“DBMP”s) (FIG. 123 a),b)) (Mikolajczyk et al., “A New General Synthesis of Ketene Thioacetals,” Tetrahedron Lett. 17:2731-2734 (1976); Kruse et al., “Synthetic Applications of 2-chloro-1,3-dithiane Preparation of Ketene Dithioacetals,” Tetrahedron Lett. 18:885-888 (1977)). Alternatively, these compounds may be prepared from 2-trimethylsilyl-1,3-dithiane via Peterson olefination (FIG. 123, c)) (Aggarwal et al., “Highly Enantioselective Oxidations of Ketene Dithioacetals Leading to Trans Bis-sulfoxides,” J. Org. Chem. 68:4087-4090 (2003)), from chloroalkyl alkanedithioates (FIG. 123, d)) (Meijer, “Preparation of Chloroalkyl Alkanedithioates and Their Conversion Into 2-alkylidene-1,3-dithiacycloalkanes,” Recl. Trav. Chim. Pays-Bas 94:83 (1975)), from α-chlorodithioacetals (Bellesia et al., “Ketene Thioacetals from α-Chloromercaptals,” Synth. Commun. 23:3179-3184 (1993)), or from the corresponding 1,1-dibromoalkenes and the appropriate thiol using excess DBU in DMSO (FIG. 123, e)) (Jin et al., “Room-Temperature and Metal-Free Synthesis of 1,1-Dithio-1-alkenes from 1,1-Dibromo-1-alkenes and Thiols,” Synlett 2011:2886-2890 (2011)).

While the HWE method for forming KDTAs is attractive due to its simplicity and generally high yields, DBMPs are not readily available. Even the simplest DBMP, dimethyl bis(methylthio)methylphosphonate, is not readily commercially available, and must be synthesized by the Arbuzov reaction of triethyl phosphite with bis(methylthio)chloromethane, the latter of which also lacks commercial availability (Abell & Taylor, “Dimethyl Bis(methylthio)methylphosphonate,” in Encyclopedia of Reagents for Organic Synthesis, doi:10.1002/047084289X.rd313 (2001)). DBMPs have also been synthesized previously from methylthiomethylphosphonates, and there is a single instance of a multi-step, one-pot reaction of the synthesis of diethyl bis(phenylthio)methylphosphonate from diethyl methylphosphonate, produced by alternating the addition of butyllithium and diphenyl disulfide (Mikolajczyk et al., “Sulphenylation of Phosphonates. A Facile Synthesis of α-phosphoryl Sulphides and S,S-acetals of Oxomethanephosphonates,” Synthesis 1980:127-129 (1980)). The latter approach to the synthesis of DBMPs is attractive because methylphosphonates are readily available, and avoid the necessity of synthesizing chlorodithioacetals.

The present application is directed to overcoming deficiencies in the art.

### SUMMARY

One aspect of the present application relates to a compound of formula (I) having the following structure:

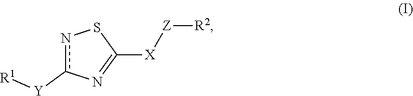

or a stereoisomer, salt, oxide, or solvate thereof, wherein

Y is a bond; —(CH₂)ₙ—; or —CH═;

R¹ is phenyl optionally substituted one or more times with halogen, CF₃, alkoxy, C₁-C₆ alkyl, NO₂, —OCF₃, —OCF₂H, —CN, alkylamine, or alkylthio; pyridine; pyrazine; carboxyalkyl; —CONH₂; alkoxy; alkylsulfide; alkylamine; benzylpiperazine; H; CF₃; and O- and N^{α}-protected amino acids comprising a carboxylate protected by a first protecting group and an amino group protected by a second protecting group; and

X is O or S;

Z is —(CH₂)ₙ—;

R² is selected from the group consisting of C₁-C₆ alkyl optionally substituted one or more times with halogen, alkoxy, or NH with an amine protecting group; C₂-C₁₀ alkenyl; phenyl optionally substituted one or more times with halogen or alkoxy; —COO(CH₂)ₙCH₃; —CO(CH₂)ₙCH₃; and an alcohol; and

n is an integer selected from 0-3,

with the following provisos:

when Y is a bond, R¹ is phenyl, X is S, and n is 0, R₂ is not methyl, ethyl, or C₃ alkylene;

when Y is a bond, R¹ is phenyl, X is S, and n is 1, R² is not phenyl;

when Y is a bond, R¹ is phenyl, X is O and n is 0, R² is not methyl;

when Y is a bond, R¹ is phenyl substituted with halogen, and n is 0, R² is not methyl;

when Y is a bond, R¹ is alkylsulfide, X is S, and n is 0, R² is not ethyl; and

when Y is a bond, R¹ is methyl, X is S, and n is 0, R² is not ethyl.

Another aspect of the present application relates to a method of treating a plant or a growing media for a nematode. This method involves contacting a plant or a growing media with a compound of formula (I) having the following structure:

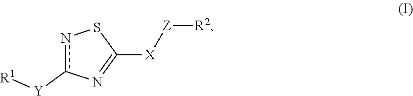

or a stereoisomer, salt, oxide, or solvate thereof, wherein

Y is a bond; —(CH₂)ₙ—; or —CH═;

R¹ is phenyl optionally substituted one or more times with halogen, CF₃, alkoxy, C₁-C₆ alkyl, NO₂, —OCF₃, —OCF₂H, —CN, alkylamine, or alkylthio; pyridine; pyrazine; carboxyalkyl; —CONH₂; alkoxy; alkylsulfide; alkylamine; benzylpiperazine; H; CF₃; and O- and N^{α}-protected amino acids comprising a carboxylate protected by a first protecting group and an amino group protected by a second protecting group; and

X is O or S;

Z is —(CH₂)ₙ—;

R² is selected from the group consisting of C₁-C₆ alkyl optionally substituted one or more times with halogen, alkoxy, or NH with an amine protecting group; C₂-C₁₀ alkenyl; phenyl optionally substituted one or more times with halogen or alkoxy; —COO(CH₂)ₙCH₃; —CO(CH₂)ₙCH₃; and an alcohol; and

n is an integer selected from 0-3,

to treat the plant or growing media for a nematode.

A further aspect of the present application relates to a composition comprising a compound of formula (I) and an agriculturally acceptable carrier.

Another aspect of the present application relates to a compound of formula (II) having the following structure:

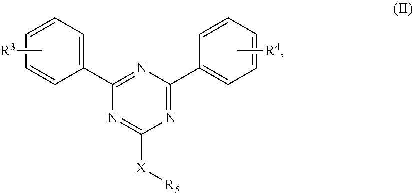

or a stereoisomer, salt, oxide, or solvate thereof, wherein

R³ and R⁴ are independently present or absent, and when present are independently halogen, alkoxy, or C₁-C₆ alkyl;

X is O or S; and

R⁵ is C₁-C₆ alkyl.

A further aspect of the present application relates to a method of treating a plant or a growing media for a nematode. This method involves contacting a plant or a growing media with a compound of formula (II) described herein to treat the plant or growing media for a nematode.

Another aspect of the present application relates to a composition comprising a compound of formula (II) as described herein and an agriculturally acceptable carrier.

A further aspect of the present application relates to a compound of formula (III) having the following structure:

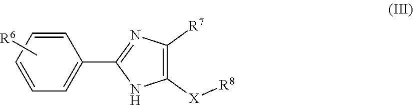

or a stereoisomer, salt, oxide, or solvate thereof, wherein

R⁶ is present or absent and when present is a halogen, alkoxy, or C₁-C₆ alkyl;

R⁷ is selected from H, C₁-C₆ alkyl, alkoxy, and —COO(CH₂)ₙCH₃;

X is S or O;

R⁸ is —(CH₂)ₙCOO(CH₂)ₙCH₃; and

n is an integer between 0-3.

Another aspect of the present application relates to a method of treating a plant or a growing media for a nematode. This method involves contacting a plant or a growing media with a compound for formula (III) to treat the plant or growing media for a nematode.

A further aspect of the present application relates to a composition comprising a compound of formula (III) as described herein and an agriculturally acceptable carrier.

Another aspect of the present application relates to a method of making a compound formula (I) having the following structure:

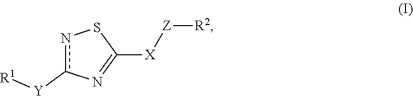

or a stereoisomer, salt, oxide, or solvate thereof, wherein

Y is a bond; —(CH₂)ₙ—; or —CH═;

R¹ is phenyl optionally substituted one or more times with halogen, CF₃, alkoxy, C₁-C₆ alkyl, NO₂, —OCF₃, —OCF₂H, —CN, alkylamine, or alkylthio; pyridine; pyrazine; carboxyalkyl; —CONH₂; alkoxy; alkylsulfide; alkylamine; benzylpiperazine; H; CF₃; and O- and N^{α}-protected amino acids comprising a carboxylate protected by a first protecting group and an amino group protected by a second protecting group; and

X is O or S;

Z is —(CH₂)ₙ—;

R² is selected from the group consisting of C₁-C₆ alkyl optionally substituted one or more times with halogen, alkoxy, or NH with an amine protecting group; C₂-C₁₀ alkenyl; phenyl optionally substituted one or more times with halogen or alkoxy; —COO(CH₂)ₙCH₃; —CO(CH₂)ₙCH₃; and an alcohol; and

n is an integer selected from 0-3. This method involves providing a starting material comprising an amidine, isourea, guanidine, or isothiourea molecule; reacting the starting material with carbon disulfide or an alkyl imidazole-1-carbodithioate molecule to form a dithiocarbamate compound; and converting the dithiocarbamate compound to a compound of formula (I).

For the synthesis of ATTDs to be competitive with this literature method, the simplicity of a mild, one-pot reaction, building the heterocyclic ring from amidines, readily accessible from a variety of methods (Boere et al., “Preparation of N,N,N′-tris(trimethylsilyl)amidines; A Convenient Route to Unsubstituted Amidines,” J. Organomet. Chem. 331:161-167 (1987); Pinner & Klein, “Umwandlung der Nitrile in Imide,” Ber. Dtsch. Chem. Ges. 10:1889-1897 (1877); Sahay et al., “Revisiting Aryl Amidine Synthesis Using Metal Amide and/or Ammonia Gas: Novel Molecules and Their Biological Evaluation,” Synth. Commun. 47:1400-1408 (2017), which are hereby incorporated by reference in their entirety), and carbon disulfide was prioritized. Furthermore, it was also desirable to keep the reaction time relatively short under mild conditions, while keeping yields comparable to previous approaches. Here, such a one-pot synthesis, performed at room temperature and in under 24 hours, is described, producing ATTDs in modest to good yields (FIG. 2, d)).

A further aspect of the present application relates to a compound of formula (IV) having the following structure:

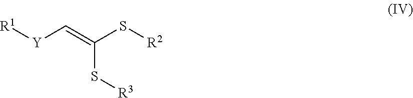

or a stereoisomer, salt, oxide, or solvate thereof, wherein

Y is a bond or C₂-C₁₀ alkylene;

R¹ is optional, and when present is phenyl optionally substituted at one or more positions with halogen, CF₃, NO₂, —CN, C₁-C₆ alkyl, C₁-C₆ alkoxy optionally substituted one or more times with halogen, C₁-C₆ alkylthio, —SCF₃, C₁-C₆ alkylamine; benzodioxolyl optionally substituted one or more times with halogen; naphthalene; thiophene; indole optionally substituted one or more times with C₁-C₆ alkyl; and pyridine optionally substituted one or more times with halogen; and

R² and R³ are independently selected from C₁-C₈ alkyl and phenyl,

with the following provisos:

when Y is a bond and R¹ is phenyl or phenyl substituted one time with a halogen, NO₂ or MeO, R² and R³ are not ethyl;

when Y is a bond and R¹ is naphthalene, R² and R³ are not methyl; and

when Y is C₂ alkylene and R¹ is phenyl, R² and R³ are not ethyl.

Another aspect of the present application relates to a method of treating a plant or growing media for a nematode. This method involves contacting a plant or growing media with a compound of formula (IV) having the following structure:

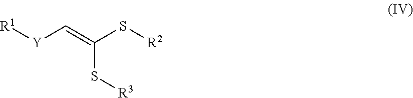

or a stereoisomer, salt, oxide, or solvate thereof, wherein

Y is a bond or C₂-C₁₀ alkylene;

R¹ is optional, and when present is phenyl optionally substituted at one or more positions with halogen, CF₃, NO₂, —CN, C₁-C₆ alkyl, C₁-C₆ alkoxy optionally substituted one or more times with halogen, C₁-C₆ alkylthio, —SCF₃, C₁-C₆ alkylamine; benzodioxolyl optionally substituted one or more times with halogen; naphthalene; thiophene; indole optionally substituted one or more times with C₁-C₆ alkyl; and pyridine optionally substituted one or more times with halogen; and

R² and R³ are independently selected from C₁-C₈ alkyl and phenyl to treat the plant or growing media for a nematode.

A further aspect of the present application relates to a method of treating a plant or growing media for a nematode. This method involves contacting a plant or growing media with a compound of formula (V) having the following structure:

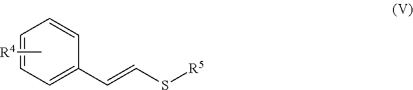

or a stereoisomer, salt, oxide, or solvate thereof, wherein

R⁴ is selected from C₁-C₆ alkyl, alkoxy, CF₃, and halogen and

R⁵ is C₁-C₆ alkyl

to treat the plant or growing media for a nematode.

Another aspect of the present application relates to a method of forming a ketene dithioacetal compound having a structure of formula (VI)

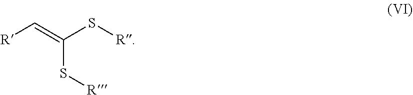

This method involves providing a dimethyl methyl phosphonate compound having a structure of

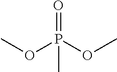

and
reacting the dimethyl methyl phosphonate compound with a disulfide compound and an aldehyde to produce the compound of formula (VI), wherein R′, R″, and R′″ are any compatible substituent.

A further aspect of the present application relates to a composition comprising a compound of formula (IV) as described herein and an agriculturally acceptable carrier.

By simplifying the one-pot synthesis of DBMPs over the previous procedure, the work herein allows for the synthesis of KDTAs from dimethyl methylphosphonate (1), disulfides, and aldehydes in one pot.

### BRIEF DESCRIPTION OF THE DRAWINGS

FIG. 1 is a schematic illustration showing illustrative examples of previously reported 1,2,4-thiadiazoles.

FIG. 2 is a schematic illustration of Scheme 1 showing synthesis of 5-alkylthiodiazoles from amidines and amidoximes.

FIG. 3 is a schematic illustration of Scheme 2 showing synthesis of compound 4a from benzamidine using ethyl imidazole-1-carbodithioate.

FIG. 4 is a schematic illustration showing the scope and yields of one embodiment of the one-pot synthesis of ATTDs. Reactions were performed with 1 mmol amidine hydrochloride and an alkyl bromide except where noted; percentages refer to isolated yields. ^{a}Yield in parentheses refers to yield using ethyl iodide instead of ethyl bromide. ^{b}Alkyl iodide used instead of alkyl halide. ^{c}Performed on a 5-mmol scale. ^{d}Hemisulfate salt used. ^{e}Yield in parentheses refers to yield in 10% DMPU in ACN was used as the solvent. ^{f}Mixture of products obtained; no yield recorded. ^{g}Hydrobromide salt used. ^{h}Hydroiodide salt used. ⁱAcetate salt used. ^{j}Free base used.

FIG. 5 is a schematic illustration of Scheme 3 showing DBU-mediated scrambling in dialkyl (dithiocarboxy)isothiourea to produce a mixture of bis(alkylthio)-1,2,4-thiadiazoles.

FIG. 6 is a schematic illustration of Scheme 4 showing formation of imidazole 6 from a thiadiazonium salt.

FIG. 7 is a schematic illustration of Scheme 5 showing addition of electrophiles other than alkyl halides to the 1c-CS₂ adduct.

FIG. 8 is a schematic illustration of Scheme 6 showing proposed routes to observed 1,3,5-triazines (11b and 14) and 5-methoxy-1,2,4-thiadiazoles (10b) (R=4-chlorophenyl).

FIG. 9 is a schematic illustration of Scheme 7 showing synthesis of 5-ethoxy-3-phenyl-1,2,4-thiadiazole (15). Conditions: DBU (2.05 eq), CS₂ (1.5 eq) and 1 (1 eq) in 1:1 acetonitrile:ethanol, 22° C., 120 min; then EtBr (1.5 eq), 22° C., 6 hr, then NCS (1.1 eq), 0° C., 30 min.

FIG. 10 is a graph showing ¹H NMR (400 MHz, CDCl₃) data for ethyl 1-imidazolecarbodithioate (2).

FIG. 11 is a graph showing ¹³C NMR (101 MHz, CDCl₃) data for ethyl 1-imidazolecarbodithioate (2).

FIG. 12 is a graph showing ¹H NMR (400 MHz, CDCl₃) data for S-Ethyl N-(α-iminobenzyl)dithiocarbamate (3a).

FIG. 13 is a graph showing ¹H NMR (400 MHz, DMSO-d₆) data for S-Ethyl N-(α-iminobenzyl)dithiocarbamate (3a).

FIG. 14 is a graph showing ¹³C NMR (101 MHz, CDCl₃) data for S-Ethyl N-(α-iminobenzyl)dithiocarbamate (3a).

FIG. 15 is a graph showing ¹³C NMR (101 MHz, DMSO-d₆) data for S-Ethyl N-(α-iminobenzyl)dithiocarbamate (3a).

FIG. 16 is a graph showing ¹H NMR (600 MHz, CDCl₃) data for S-Ethyl N-(α-imino-4-chlorobenzyl)dithiocarbamate (3b).

FIG. 17 is a graph showing ¹H NMR (600 MHz, CDCl₃) data for S-Ethyl N-(α-imino-4-chlorobenzyl)dithiocarbamate (3b).

FIG. 18 is a graph showing ¹H NMR (400 MHz, CDCl₃) data for 5-Ethylthio-3-phenyl-1,2,4-thiadiazole (4a).

FIG. 19 is a graph showing ¹³C NMR (101 MHz, CDCl₃) data for 5-Ethylthio-3-phenyl-1,2,4-thiadiazole (4a).

FIG. 20 is a graph showing ¹H NMR (400 MHz, CDCl₃) data for 5-Methylthio-3-phenyl-1,2,4-thiadiazole (4b).

FIG. 21 is a graph showing ¹³C NMR (101 MHz, CDCl₃) data for 5-Methylthio-3-phenyl-1,2,4-thiadiazole (4b).

FIG. 22 is a graph showing ¹H NMR (400 MHz, CDCl₃) data for 3-Phenyl-5-propylthio-1,2,4-thiadiazole (4c).

FIG. 23 is a graph showing ¹³C NMR (101 MHz, CDCl₃) data for 3-Phenyl-5-propylthio-1,2,4-thiadiazole (4c).

FIG. 24 is a graph showing ¹H NMR (400 MHz, CDCl₃) data for 5-Isopropylthio-3-phenyl-1,2,4-thiadiazole (4d).

FIG. 25 is a graph showing ¹³C NMR (101 MHz, CDCl₃) data for 5-Isopropylthio-3-phenyl-1,2,4-thiadiazole (4d).

FIG. 26 is a graph showing ¹H NMR (400 MHz, CDCl₃) data for 5-Isobutylthio-3-phenyl-1,2,4-thiadiazole (4e).

FIG. 27 is a graph showing ¹H NMR (400 MHz, CDCl₃) data for 5-(Cyclopropylmethylthio)-3-phenyl-1,2,4-thiadiazole (4f).

FIG. 28 is a graph showing ¹H NMR (400 MHz, CDCl₃) data for 5-(Cyclopropylmethylthio)-3-phenyl-1,2,4-thiadiazole (4f).

FIG. 29 is a graph showing ¹³C NMR (101 MHz, CDCl₃) data for 5-(Cyclopropylmethylthio)-3-phenyl-1,2,4-thiadiazole (4f).

FIG. 30 is a graph showing ¹H NMR (400 MHz, CDCl₃) data for 5-Allylthio-3-phenyl-1,2,4-thiadiazole (4g).

FIG. 31 is a graph showing ¹³C NMR (101 MHz, CDCl₃) data for 5-Allylthio-3-phenyl-1,2,4-thiadiazole (4g).

FIG. 32 is a graph showing ¹H NMR (400 MHz, CDCl₃) data for 5-Benzylthio-3-phenyl-1,2,4-thiadiazole (4h).

FIG. 33 is a graph showing ¹³C NMR (101 MHz, CDCl₃) data for 5-Benzylthio-3-phenyl-1,2,4-thiadiazole (4h).

FIG. 34 is a graph showing ¹H NMR (600 MHz, CDCl₃) data for 5-Citronellylthio-3-phenyl-1,2,4-thiadiazole (4i).

FIG. 35 is a graph showing ¹³C NMR (151 MHz, CDCl₃) data for 5-Citronellylthio-3-phenyl-1,2,4-thiadiazole (4i).

FIG. 36 is a graph showing ¹H NMR (400 MHz, CDCl₃) data for 5-Ethylthio-3-(4-fluorophenyl)-1,2,4-thiadiazole (4j).

FIG. 37 is a graph showing ¹³C NMR (101 MHz, CDCl₃) data for 5-Ethylthio-3-(4-fluorophenyl)-1,2,4-thiadiazole (4j).

FIG. 38 is a graph showing ¹⁹F NMR (376 MHz, CDCl₃) data for 5-Ethylthio-3-(4-fluorophenyl)-1,2,4-thiadiazole (4j).

FIG. 39 is a graph showing ¹H NMR (400 MHz, CDCl₃) data for 3-(4-Chlorophenyl)-5-ethylthio-1,2,4-thiadiazole (4k).

FIG. 40 is a graph showing ¹³C NMR (101 MHz, CDCl₃) data for 3-(4-Chlorophenyl)-5-ethylthio-1,2,4-thiadiazole (4k).

FIG. 41 is a graph showing ¹H NMR (400 MHz, CDCl₃) data for 3-(4-Chlorophenyl)-5-methylthio-1,2,4-thiadiazole (4l).

FIG. 42 is a graph showing ¹³C NMR (101 MHz, CDCl₃) data for 3-(4-Chlorophenyl)-5-methylthio-1,2,4-thiadiazole (4l).

FIG. 43 is a graph showing ¹H NMR (600 MHz, CDCl₃) data for 3-(4-Chlorophenyl)-5-(3-chloroprop-1-yl)-1,2,4-thiadiazole (4m).

FIG. 44 is a graph showing ¹³C NMR (151 MHz, CDCl₃) data for 3-(4-Chlorophenyl)-5-(3-chloroprop-1-yl)-1,2,4-thiadiazole (4m).

FIG. 45 is a graph showing ¹H NMR (600 MHz, CDCl₃) data for 5-((2-bromo-5-methoxybenzyl)thio)-3-(4-chlorophenyl)-1,2,4-thiadiazole (4n).

FIG. 46 is a graph showing ¹³C NMR (151 MHz, CDCl₃) data for 5-((2-bromo-5-methoxybenzyl)thio)-3-(4-chlorophenyl)-1,2,4-thiadiazole (4n).

FIG. 47 is a graph showing ¹H NMR (400 MHz, CDCl₃) data for 3-(4-Bromophenyl)-5-ethylthio-1,2,4-thiadiazole (4o).

FIG. 48 is a graph showing ¹³C NMR (101 MHz, CDCl₃) data for 3-(4-Bromophenyl)-5-ethylthio-1,2,4-thiadiazole (4o).

FIG. 49 is a graph showing ¹H NMR (400 MHz, CDCl₃) data for 5-Ethylthio-3-(4-(trifluoromethyl)phenyl)-1,2,4-thiadiazole (4p).

FIG. 50 is a graph showing ¹³C NMR (101 MHz, CDCl₃) data for 5-Ethylthio-3-(4-(trifluoromethyl)phenyl)-1,2,4-thiadiazole (4p).

FIG. 51 is a graph showing ¹⁹F NMR (376 MHz, CDCl₃) data for 5-Ethylthio-3-(4-(trifluoromethyl)phenyl)-1,2,4-thiadiazole (4p).

FIG. 52 is a graph showing ¹H NMR (400 MHz, CDCl₃) data for 5-Ethylthio-3-(4-methoxyphenyl)-1,2,4-thiadiazole (4q).

FIG. 53 is a graph showing ¹³C NMR (101 MHz, CDCl₃) data for 5-Ethylthio-3-(4-methoxyphenyl)-1,2,4-thiadiazole (4q).

FIG. 54 is a graph showing ¹H NMR (400 MHz, CDCl₃) data for 5-Ethylthio-3-(3-methoxyphenyl)-1,2,4-thiadiazole (4r).

FIG. 55 is a graph showing ¹³C NMR (101 MHz, CDCl₃) data for 5-Ethylthio-3-(3-methoxyphenyl)-1,2,4-thiadiazole (4r).

FIG. 56 is a graph showing ¹H NMR (600 MHz, CDCl₃) data for 5-((2-Methoxyethyl)thio)-3-(3-methoxyphenyl)-1,2,4-thiadiazole (4s).

FIG. 57 is a graph showing ¹³C NMR (151 MHz, CDCl₃) data for 5-((2-Methoxyethyl)thio)-3-(3-methoxyphenyl)-1,2,4-thiadiazole (4s).

FIG. 58 is a graph showing ¹H NMR (600 MHz, CDCl₃) data for tert-Butyl (2-((3-phenyl-1,2,4-thiadiazol-5-yl)thio)ethyl)carbamate (4t).

FIG. 59 is a graph showing ¹³C NMR (151 MHz, CDCl₃) data for tert-Butyl (2-((3-phenyl-1,2,4-thiadiazol-5-yl)thio)ethyl)carbamate (4t).

FIG. 60 is a graph showing ¹H NMR (400 MHz, CDCl₃) data for 5-Ethylthio-3-(3-toluyl)-1,2,4-thiadiazole (4u).

FIG. 61 is a graph showing ¹³C NMR (101 MHz, CDCl₃) data for 5-Ethylthio-3-(3-toluyl)-1,2,4-thiadiazole (4u).

FIG. 62 is a graph showing ¹H NMR (400 MHz, CDCl₃) data for 5-Ethylthio-3-(4-nitrophenyl)-1,2,4-thiadiazole (4v).

FIG. 63 is a graph showing ¹³C NMR (101 MHz, CDCl₃) data for 5-Ethylthio-3-(4-nitrophenyl)-1,2,4-thiadiazole (4v).

FIG. 64 is a graph showing ¹H NMR (400 MHz, CDCl₃) data for 5-Ethylthio-3-(pyridin-2-yl)-1,2,4-thiadiazole (4w).

FIG. 65 is a graph showing ¹³C NMR (101 MHz, CDCl₃) data for 5-Ethylthio-3-(pyridin-2-yl)-1,2,4-thiadiazole (4w).

FIG. 66 is a graph showing ¹H NMR (400 MHz, CDCl₃) data for 5-Ethylthio-3-(pyridin-4-yl)-1,2,4-thiadiazole (4x).

FIG. 67 is a graph showing ¹³C NMR (101 MHz, CDCl₃) data for 5-Ethylthio-3-(pyridin-4-yl)-1,2,4-thiadiazole (4x).

FIG. 68 is a graph showing ¹H NMR (400 MHz, CDCl₃) data for 5-Ethylthio-3-(pyrazin-2-yl)-1,2,4-thiadiazole (4y).

FIG. 69 is a graph showing ¹³C NMR (101 MHz, CDCl₃) data for 5-Ethylthio-3-(pyrazin-2-yl)-1,2,4-thiadiazole (4y).

FIG. 70 is a graph showing ¹H NMR (400 MHz, CDCl₃) data for 5-Ethylthio-3-methyl-1,2,4-thiadiazole (5a).

FIG. 71 is a graph showing ¹³C NMR (101 MHz, CDCl₃) data for 5-Ethylthio-3-methyl-1,2,4-thiadiazole (5a).

FIG. 72 is a graph showing ¹H NMR (400 MHz, CDCl₃) data for 3-Cyclopropyl-5-ethylthio-1,2,4-thiadiazole (5b).

FIG. 73 is a graph showing ¹³C NMR (101 MHz, CDCl₃) data for 3-Cyclopropyl-5-ethylthio-1,2,4-thiadiazole (5b).

FIG. 74 is a graph showing ¹H NMR (400 MHz, CDCl₃) data for 3-(2,6-Dichlorobenzyl)-5-ethylthio-1,2,4-thiadiazole (5c).

FIG. 75 is a graph showing ¹³C NMR (101 MHz, CDCl₃) data for 3-(2,6-Dichlorobenzyl)-5-ethylthio-1,2,4-thiadiazole (5c).

FIG. 76 is a graph showing ¹H NMR (400 MHz, CDCl₃) data for Ethyl 2-(5-ethylthio-1,2,4-thiadiazol-3-yl)acetate (5d).

FIG. 77 is a graph showing ¹³C NMR (101 MHz, CDCl₃) data for Ethyl 2-(5-ethylthio-1,2,4-thiadiazol-3-yl)acetate (5d).

FIG. 78 is a graph showing ¹H NMR (400 MHz, CDCl₃) data for 2-(5-(Ethylthio)-1,2,4-thiadiazol-3-yl)acetamide (5e).

FIG. 79 is a graph showing ¹³C NMR (101 MHz, CDCl₃) data for 2-(5-(Ethylthio)-1,2,4-thiadiazol-3-yl)acetamide (5e).

FIG. 80 is a graph showing ¹H NMR (400 MHz, CDCl₃) data for 5-Ethylthio-3-methoxy-1,2,4-thiadiazole (5f).

FIG. 81 is a graph showing ¹³C NMR (101 MHz, CDCl₃) data for 5-Ethylthio-3-methoxy-1,2,4-thiadiazole (5f).

FIG. 82 is a graph showing ¹H NMR (400 MHz, CDCl₃) data for 3,5-Bis(ethylthio)-1,2,4-thiadiazole (5h).

FIG. 83 is a graph showing ¹³C NMR (101 MHz, CDCl₃) data for 3,5-Bis(ethylthio)-1,2,4-thiadiazole (5h).

FIG. 84 is a graph showing ¹H NMR (400 MHz, CDCl₃) data for 5-Ethylthio-3-dimethylamino-1,2,4-thiadiazole (5i).

FIG. 85 is a graph showing ¹³C NMR (101 MHz, CDCl₃) data for 5-Ethylthio-3-dimethylamino-1,2,4-thiadiazole (5i).

FIG. 86 is a graph showing ¹H NMR (600 MHz, CDCl₃) data for 3-(4-Benzylpiperazin-1-yl)-5-ethylthio-1,2,4-thiadiazole (5j).

FIG. 87 is a graph showing ¹³C NMR (151 MHz, CDCl₃) data for 3-(4-Benzylpiperazin-1-yl)-5-ethylthio-1,2,4-thiadiazole (5j).

FIG. 88 is a graph showing ¹H NMR (400 MHz, CDCl₃) data for N^{α}-Benzoyl-O ethyl-N^{δ}-(5-ethylthio-1,2,4-thiadiazol-3-yl)ornithine (5k).

FIG. 89 is a graph showing ¹H NMR (400 MHz, CDCl₃) data for N^{α}-Benzoyl-O-ethyl-N^{δ}-(5-ethylthio-1,2,4-thiadiazol-3-yl)ornithine (5k).

FIG. 90 is a graph showing HMQC (400 MHz, CDCl₃) data for N^{α}-Benzoyl-O-ethyl-N^{δ}-(5-ethylthio-1,2,4-thiadiazol-3-yl)ornithine (5k).

FIG. 91 is a graph showing ¹H NMR (400 MHz, CDCl₃) data for 3-Phenyl-5-methoxy-1,2,4-thiadiazole (10a).

FIG. 92 is a graph showing ¹³C NMR (101 MHz, CDCl₃) data for 3-Phenyl-5-methoxy-1,2,4-thiadiazole (10a).

FIG. 93 is a graph showing ¹H NMR (400 MHz, CDCl₃) data for 3-(4-Fluorophenyl)-5-methoxy-1,2,4-thiadiazole (10b).

FIG. 94 is a graph showing ¹³C NMR (101 MHz, CDCl₃) data for 3-(4-Fluorophenyl)-5-methoxy-1,2,4-thiadiazole (10b).

FIG. 95 is a graph showing ¹⁹F NMR (379 MHz, CDCl₃) data for 3-(4-Fluorophenyl)-5-methoxy-1,2,4-thiadiazole (10b).

FIG. 96 is a graph showing ¹H NMR (600 MHz, CDCl₃) data for 3-(4-Chlorophenyl)-5-methoxy-1,2,4-thiadiazole (10c).

FIG. 97 is a graph showing ¹³C NMR (151 MHz, CDCl₃) data for 3-(4-Chlorophenyl)-5-methoxy-1,2,4-thiadiazole (10c).

FIG. 98 is a graph showing ¹H NMR (600 MHz, CDCl₃) data for 5-Methoxy-3-(3-methoxyphenyl)-1,2,4-thiadiazole (10d).

FIG. 99 is a graph showing ¹³C NMR (151 MHz, CDCl₃) data for 5-Methoxy-3-(3-methoxyphenyl)-1,2,4-thiadiazole (10d).

FIG. 100 is a graph showing ¹H NMR (600 MHz, CDCl₃) data for 5-Methoxy-3-(4-nitrophenyl)-1,2,4-thiadiazole (10e).

FIG. 101 is a graph showing ¹³C NMR (151 MHz, CDCl₃) data for 5-Methoxy-3-(4-nitrophenyl)-1,2,4-thiadiazole (10e).

FIG. 102 is a graph showing ¹H NMR (400 MHz, CDCl₃) data for. 2-(4-Chlorophenyl)-4-carbmethoxy-5-((carbmethoxymethyl)thio)imidazole (6)

FIG. 103 is a graph showing ¹³C NMR (101 MHz, CDCl₃) data for 2-(4-Chlorophenyl)-4-carbmethoxy-5-((carbmethoxymethyl)thio)imidazole (6).

FIG. 104 is a graph showing ¹H NMR (400 MHz, CDCl₃) data for 5-(2-(Carbmethoxy)ethylthio)-3-(4-chlorophenyl)-1,2,4-thiadiazole (9a).

FIG. 105 is a graph showing ¹³C NMR (101 MHz, CDCl₃) data for 5-(2-(Carbmethoxy)ethylthio)-3-(4-chlorophenyl)-1,2,4-thiadiazole (9a).

FIG. 106 is a graph showing ¹H NMR (600 MHz, CDCl₃) data for 5-((3-Oxobut-1-yl)thio)-3-(4-chlorophenyl)-1,2,4-thiadiazole (9b).

FIG. 107 is a graph showing ¹³C NMR (151 MHz, CDCl₃) data for 5-((3-Oxobut-1-yl)thio)-3-(4-chlorophenyl)-1,2,4-thiadiazole (9b).

FIG. 108 is a graph showing ¹H NMR (400 MHz, CDCl₃) data for 5-(2-Hydroxy-1-butylthio)-3-(4-chlorophenyl)-1,2,4-thiadiazole (9c).

FIG. 109 is a graph showing ¹³C NMR (101 MHz, CDCl₃) data for 5-(2-Hydroxy-1-butylthio)-3-(4-chlorophenyl)-1,2,4-thiadiazole (9c).

FIG. 110 is a graph showing ¹H NMR (400 MHz, CDCl₃) data for 2-Methoxy-4,6-diphenyl-1,3,5-triazine (11a).

FIG. 111 is a graph showing ¹³C NMR (101 MHz, CDCl₃) data for 2-Methoxy-4,6-diphenyl-1,3,5-triazine (11a).

FIG. 112 is a graph showing ¹H NMR (600 MHz, CDCl₃) data for 4,6-Bis(4-fluorophenyl)-2-methoxy-1,3,5-triazine (11b).

FIG. 113 is a graph showing ¹³C NMR (151 MHz, CDCl₃) data for 4,6-Bis(4-fluorophenyl)-2-methoxy-1,3,5-triazine (11b).

FIG. 114 is a graph showing ¹⁹F{¹H} NMR (565 MHz, CDCl₃) data for 4,6-Bis(4-fluorophenyl)-2-methoxy-1,3,5-triazine (11b).

FIG. 115 is a graph showing ¹H NMR (400 MHz, CDCl₃) data for 4,6-Bis(4-chlorophenyl)-2-methoxy-1,3,5-triazine (tic).

FIG. 116 is a graph showing ¹³C NMR (101 MHz, CDCl₃) data for 4,6-Bis(4-chlorophenyl)-2-methoxy-1,3,5-triazine (tic).

FIG. 117 is a graph showing ¹H NMR (400 MHz, CDCl₃) data for 4,6-Bis(3-methoxyphenyl)-2-methoxy-1,3,5-triazine (11d).

FIG. 118 is a graph showing ¹³C NMR (101 MHz, CDCl₃) data for 4,6-Bis(3-methoxyphenyl)-2-methoxy-1,3,5-triazine (11d).

FIG. 119 is a graph showing ¹H NMR (400 MHz, CDCl₃) data for 4,6-Bis(4-chlorophenyl)-2-ethylthio-1,3,5-triazine (14).

FIG. 120 is a graph showing ¹³C NMR (101 MHz, CDCl₃) data for 4,6-Bis(4-chlorophenyl)-2-ethylthio-1,3,5-triazine (14).

FIG. 121 is a graph showing ¹H NMR (400 MHz, CDCl₃) data for 3-Phenyl-5-ethoxy-1,2,4-thiadiazole (15).

FIG. 122 is a graph showing ¹³C NMR (101 MHz, CDCl₃) data for 3-Phenyl-5-ethoxy-1,2,4-thiadiazole (15).

FIG. 123 is a schematic illustration showing selected methods for the synthesis of ketene dithioacetals lacking an electron-withdrawing group on the α-carbon.

FIG. 124 is a schematic illustration of Scheme 8, showing stepwise synthesis of compound 4a from dimethyl methylphosphonate (1).

FIG. 125 is a schematic illustration of Scheme 9, showing scope and yields of the one-pot synthesis of ketenedithioacetals, according to one embodiment of the present application.

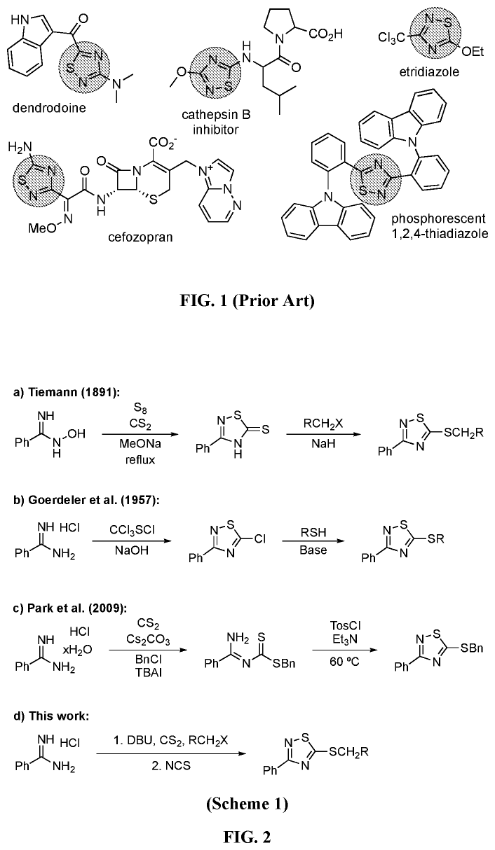

### DETAILED DESCRIPTION

The present application relates to 1,2,4-thiadiazole and ketene dithioacetal compounds as defined herein, compositions containing these compounds, methods of their use, and methods of making. The compounds have nematacide activity.

1,2,4-Thiadiazoles

One aspect of the present application relates to a compound of formula (I) having the following structure:

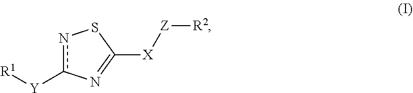

or a stereoisomer, salt, oxide, or solvate thereof, wherein

Y is a bond; —(CH₂)ₙ—; or —CH═;

R¹ is phenyl optionally substituted one or more times with halogen, CF₃, alkoxy, C₁-C₆ alkyl, NO₂, —OCF₃, —OCF₂H, —CN, alkylamine, or alkylthio; pyridine; pyrazine; carboxyalkyl; —CONH₂; alkoxy; alkylsulfide; alkylamine; benzylpiperazine; H; CF₃; and O- and N^{α}-protected amino acids comprising a carboxylate protected by a first protecting group and an amino group protected by a second protecting group; and

X is O or S;

Z is —(CH₂)ₙ—;

R² is selected from the group consisting of C₁-C₆ alkyl optionally substituted one or more times with halogen, alkoxy, or NH with an amine protecting group; C₂-C₁₀ alkenyl; phenyl optionally substituted one or more times with halogen or alkoxy; —COO(CH₂)ₙCH₃; —CO(CH₂)ₙCH₃; and an alcohol; and

n is an integer selected from 0-3,

with the following provisos:

when Y is a bond, R¹ is phenyl, X is S, and n is 0, R² is not methyl, ethyl, or C₃ alkylene;

when Y is a bond, R¹ is phenyl, X is S, and n is 1, R² is not phenyl;

when Y is a bond, R¹ is phenyl, X is O and n is 0, R² is not methyl;

when Y is a bond, R¹ is phenyl substituted with halogen, and n is 0, R² is not methyl;

when Y is a bond, R¹ is alkylsulfide, X is S, and n is 0, R² is not ethyl; and

when Y is a bond, R¹ is methyl, X is S, and n is 0, R² is not ethyl.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs.

By “compound(s) of the present application” and equivalent expressions, it is meant compounds herein described, which expression includes the prodrugs, the pharmaceutically acceptable salts, the oxides, and the solvates, e.g. hydrates, where the context so permits.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. The present application is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. All tautomeric forms are also intended to be included.

As would be understood by a person of ordinary skill in the art, the recitation of “a compound” is intended to include salts, solvates, oxides, and inclusion complexes of that compound as well as any stereoisomeric form, or a mixture of any such forms of that compound in any ratio. Thus, in accordance with some embodiments of the application, a compound as described herein, including in the contexts of agricultural compositions, methods of use, and compounds per se, is provided as the salt form.

The term “solvate” refers to a compound in the solid state, where molecules of a suitable solvent are incorporated in the crystal lattice. Examples of suitable solvents are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

As used herein, the term “halogen” means fluoro, chloro, bromo, or iodo.

The term “alkoxy” or “C₁-C₆ alkoxy” means groups of from 1 to 6 carbon atoms of a straight, branched, or cyclic configuration and combinations thereof attached to the parent structure through an oxygen. Examples include methoxy, ethoxy, propoxy, isopropoxy, butoxy, cyclopropyloxy, cyclohexyloxy, and the like. Alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

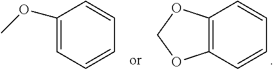

The term “alkyl” means an aliphatic hydrocarbon group which may be straight or branched having about 1 to about 6 carbon atoms in the chain (or the number of carbons designated by “Cₙ-Cₙ”, where n is the numerical range of carbon atoms). Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

As used herein, the term “alkylamine” refers to a structure in which a branched or unbranched aliphatic hydrocarbon group is connected to an amine group by a carbon-nitrogen bond (e.g., C—N bonding).

As used herein, the terms “alkylthio” or “C₁-C₆ alkylthio” and “alkylsulfide” refer to a structure in which a branched or unbranched aliphatic hydrocarbon group is connected to a sulfur atom by a carbon-sulfur bond (e.g., C—S bonding).

The term “carboxyalkyl” refers to the groups —C(O)O-alkyl and —C(O)O-substituted alkyl.

The term “alkylene” refers to a saturated, branched, or straight chain hydrocarbon group having two monovalent radical centers derived by the removal of two hydrogen atoms from the same or two different carbon atoms of a parent alkane. In one example, the divalent alkylene group is two to ten carbon atoms (C₂-C₁₀).

In one embodiment, the compound of formula (I) is a compound where Y is a bond and R¹ is phenyl.

In another embodiment, the compound of formula (I) is a compound where Y is a bond and R¹ is phenyl optionally substituted one or more times with halogen, CF₃, alkoxy, C₁-C₆ alkyl, NO₂, —OCF₃, —OCF₂H, —CN, alkylamine, or alkylthio.

In another embodiment, the compound of formula (I) is a compound where Y is a bond and R¹ is pyridine.

In one embodiment, the compound of formula (I) is a compound where Y is a bond and R¹ is pyrazine; carboxyalkyl; —CONH₂; alkoxy; alkylsulfide; alkylamine;

benzylpiperazine; H; CF₃; or O- and N-protected amino acids comprising a carboxylate protected by a first protecting group and an amino group protected by a second protecting group.

Where the compound of formula (I) has R¹ that is an O- and NO-protected amino acids comprising a carboxylate protected by a first protecting group and an amino group protected by a second protecting group, the first protecting group may be methyl, ethyl, allyl, t-butyl, benzyl, etc. and the second protecting group may be acetyl, benzoyl, benzyl, Boc, Fmoc, or other appropriate protecting group.

In one embodiment of the compound of formula (I), X is S. According to this embodiment, the compound of formula (I) may be a compound selected from

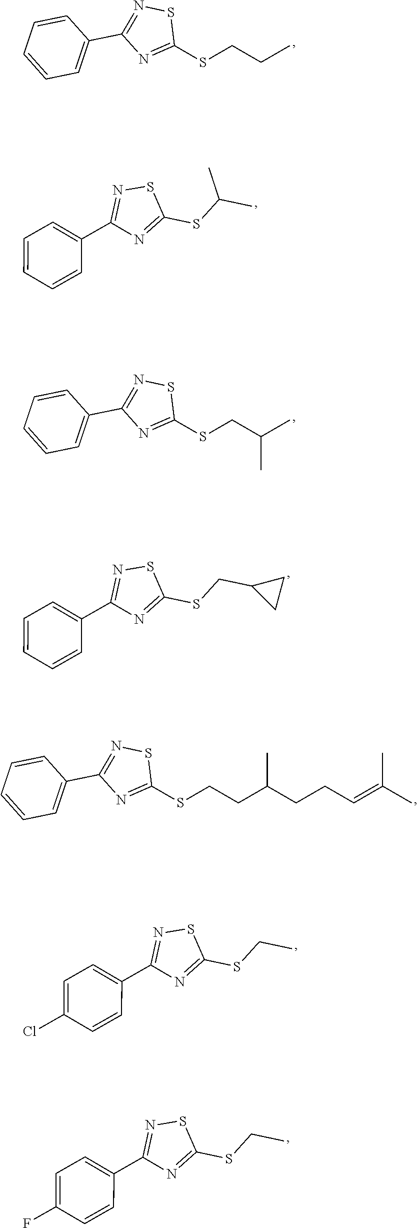

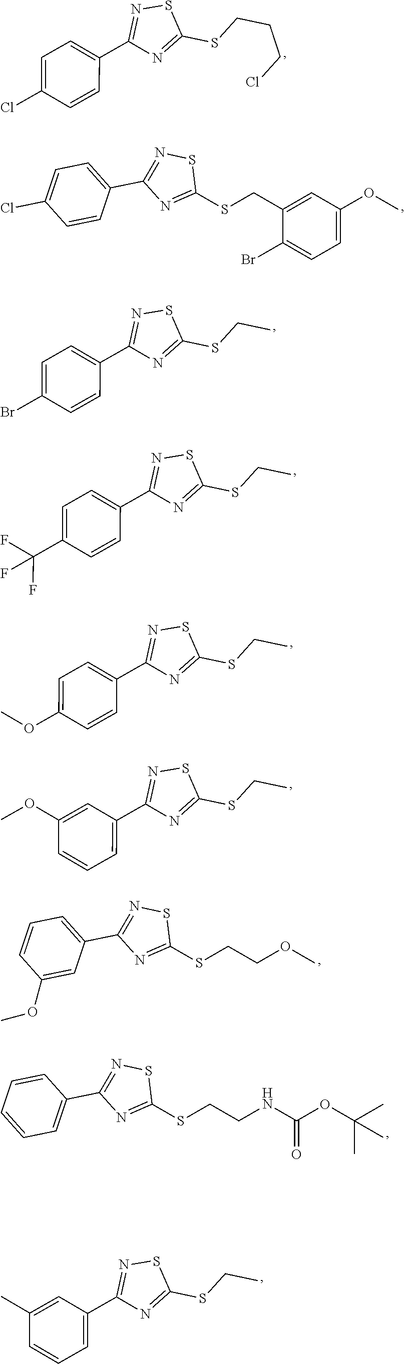

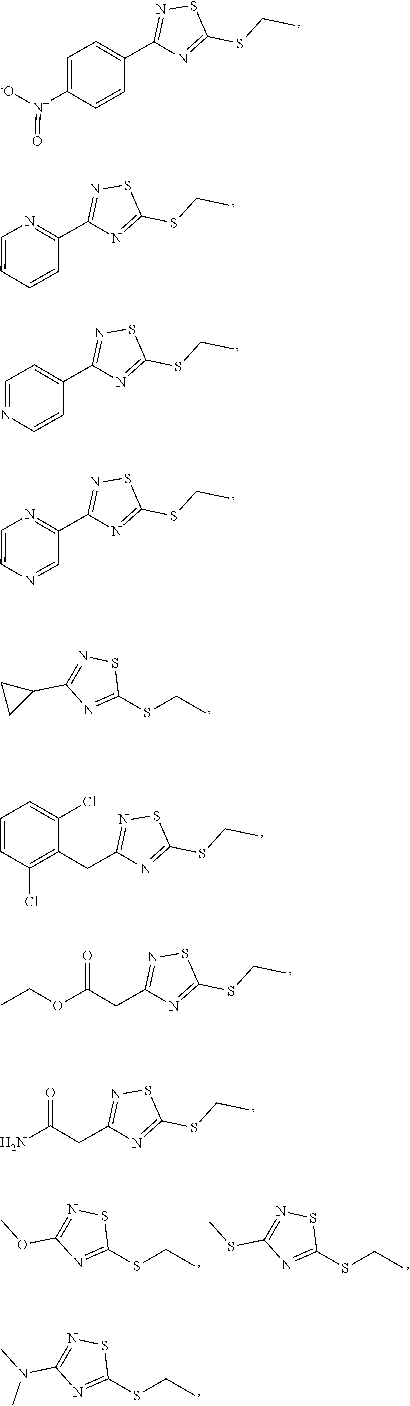

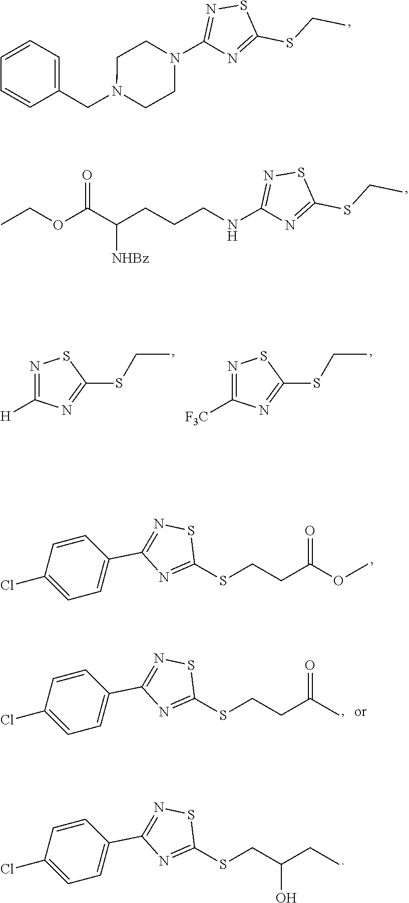

In one embodiment of the compound of formula (I), X is O. According to this embodiment, the compound of formula (I) may be a compound selected from

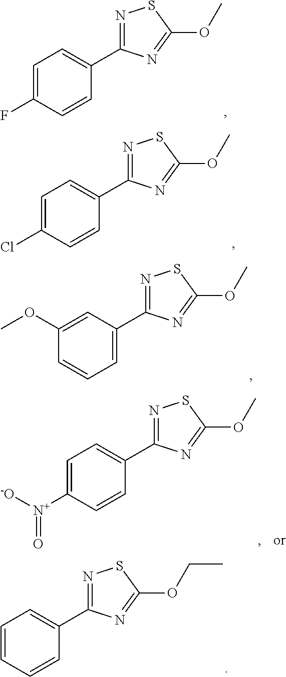

In one embodiment of the compound of formula (I), R² is C₁-C₆ alkyl.

In another embodiment of the compound of formula (I) R² is C₁-C₆ alkyl substituted with halogen, alkoxy, or NH with an amine protecting group. NH with an amine protecting group may be, e.g., NHBoc, dimethylamino, NHFmoc, or NHBz.

In one embodiment of the compound of formula (I) R² is C₂-C₁₀ alkenyl.

In one embodiment of the compound of formula (I) R² is phenyl.

In one embodiment of the compound of formula (I) R² is phenyl substituted one or more times with halogen or alkoxy.

In one embodiment of the compound of formula (I) R² is —COO(CH₂)ₙCH₃; —CO(CH₂)ₙCH₃; or an alcohol.

Another aspect of the present application relates to a method of treating a plant or a growing media for a nematode. This method involves contacting a plant or a growing media with a compound of formula (I) having the following structure:

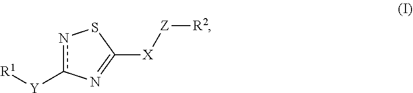

or a stereoisomer, salt, oxide, or solvate thereof, wherein

Y is a bond; —(CH₂)ₙ—; or —CH═;

R¹ is phenyl optionally substituted one or more times with halogen, CF₃, alkoxy, C₁-C₆ alkyl, NO₂, —OCF₃, —OCF₂H, —CN, alkylamine, or alkylthio; pyridine; pyrazine; carboxyalkyl; —CONH₂; alkoxy; alkylsulfide; alkylamine; benzylpiperazine; H; CF₃; and O- and N^{α}-protected amino acids comprising a carboxylate protected by a first protecting group and an amino group protected by a second protecting group; and

X is O or S;

Z is —(CH₂)ₙ—;

R² is selected from the group consisting of C₁-C₆ alkyl optionally substituted one or more times with halogen, alkoxy, or NH with an amine protecting group; C₂-C₁₀ alkenyl; phenyl optionally substituted one or more times with halogen or alkoxy; —COO(CH₂)ₙCH₃; —CO(CH₂)ₙCH₃; and an alcohol; and

n is an integer selected from 0-3,

to treat the plant or growing media for a nematode.

According to this aspect of the disclosure, a plant or a growing media is treated with a compound of formula (I).

In one embodiment, a plant is treated with a compound of formula (I).

Suitable plants amenable to the treatment methods described herein include any plant vulnerable or susceptible to nematodes. A number of genera and species of nematodes are known to be highly damaging to a great number of plant hosts, including foliage plants, agronomic and vegetable crops, fruit and nut trees, turfgrass, and forest trees.

In one embodiment, a plant treated with a compound of formula (I) is a vegetable crop. In a particular embodiment, the plant is soybean (Glycine max).

Some of the most damaging nematodes to plants include, without limitation, root-knot (Meloidogyne spp.); cyst (Heterodera and Globodera spp.); root-lesion (Pratylenchus spp.); spiral (Helicotylenchus spp.); burrowing (Radopholus similis); bulb and stem (Ditylenchus dipsaci); reniform (Rotylenchulus reniformis); dagger (Xiphinema spp.); bud and leaf (Aphelenchoides spp.); and Pine Wilt Disease (Bursaphelenchus xylophilus). According to the present disclosure, any of these (or any other plant-parasitic nematode) is treated according to the methods described herein.

In one embodiment, a growing media is treated with a compound of formula (I).

As used herein, the term “growing media” is meant to include soil or any other material in which a plant is grown or cultivated.

In carrying out methods described herein, a plant or a growing media is contacted with a compound of formula (I).

Contacting a plant or a growing media with a compound of formula (I) may involve contacting a plant or growing media with a compound of formula (I) or a composition disclosed herein, which composition contains a compound of formula (I).

Compounds of formula (I) suitable in the methods of the present application have substituents as defined herein.

In one embodiment of the compound of formula (I), X is S. According to this embodiment, the method of this aspect of the present application may be carried out with one or more compounds selected from the following structures:

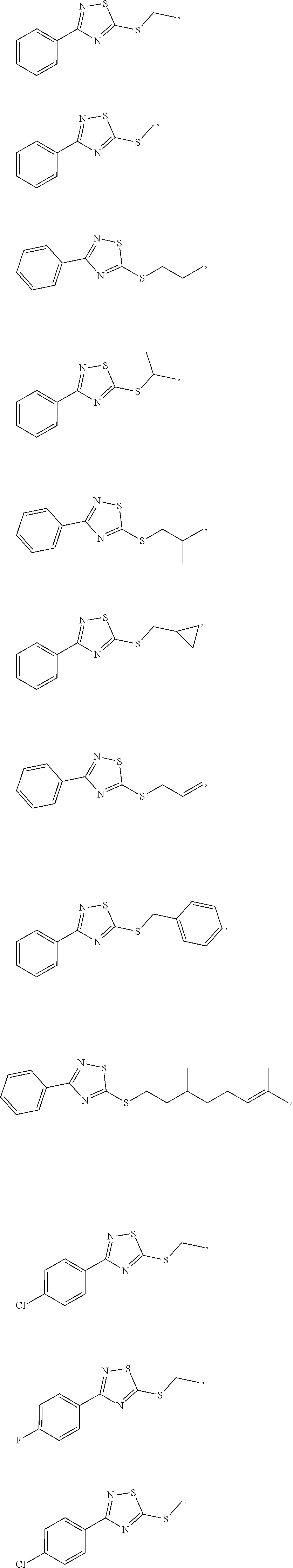

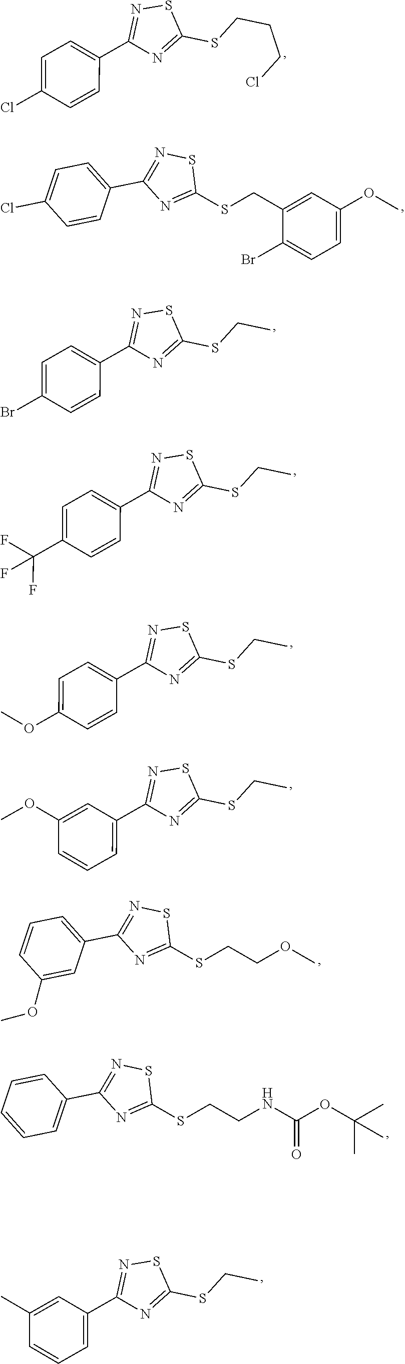

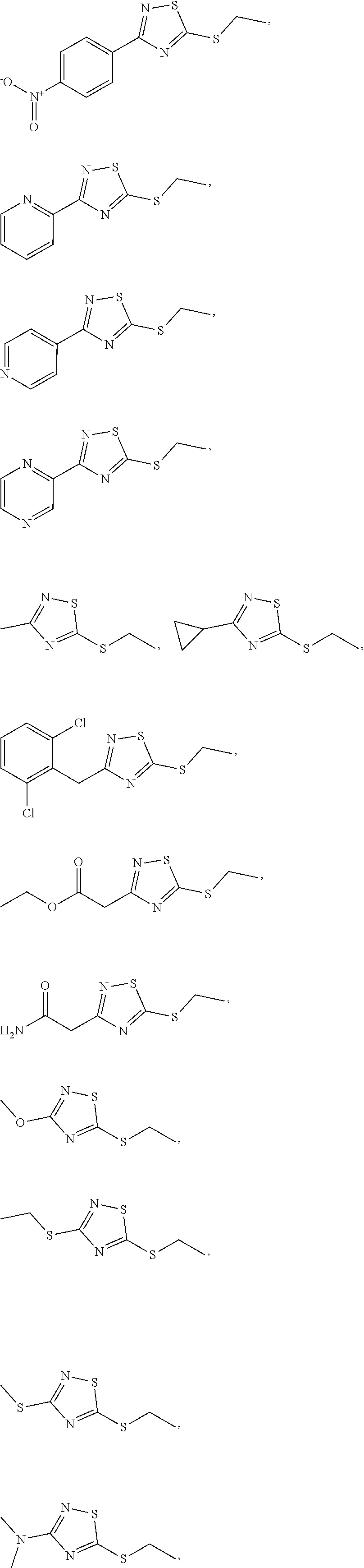

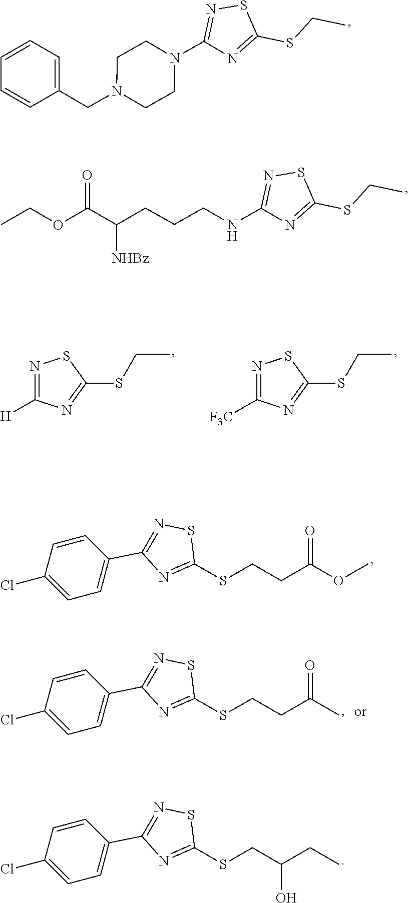

In another embodiment of the compound of formula (I), X is O. According to this embodiment, the method of this aspect of the present application may be carried out with one or more compounds selected from the following structures:

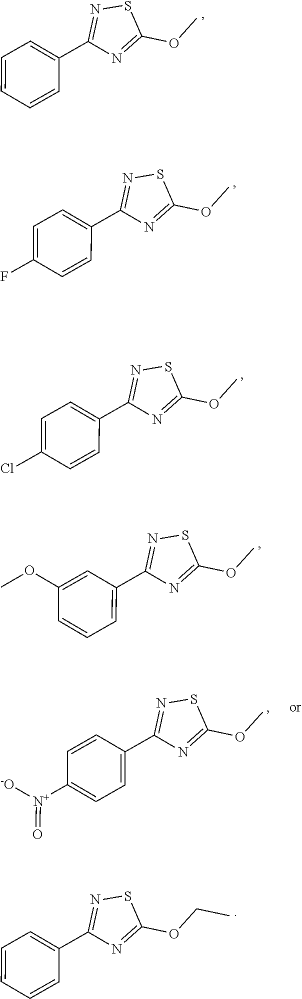

In one embodiment of carrying out said contacting, the compound of formula (I) (and other compounds described herein) is a nematacide. As used herein, the term “nematacide” means a compound that inhibits the growth of, inhibits the reproduction or reproductive cycle of, contains, prevents the growth or invasion of, or kills nematodes or nematode eggs or larvae to contain, reduce, prevent, or eliminate nematode or nematode growth or reproduction in a growing media or on a plant or a plant part.

In one embodiment, said contacting is carried out simultaneously or nearly simultaneously with planting seed in a growing media. In other words, according to one embodiment, the method is carried out simultaneously with planting a seed vulnerable (at the seed or, more likely, the plant stage) to a nematode. According to this embodiment, treatment of a growing media may happen at or near the time the seed is planted in the growing media. Alternatively, treatment of the growing media with the compound of formula (I) (or other compounds described herein) may occur via a pre-treated seed (e.g., a coating on the seed that contains a compound of formula (I) (or other compound(s) described herein), which comes into contact with the growing media to be treated at the time of planting the seed in the growing media). Seed treatment with the compound of formula (I) (or other compound(s) described herein) can be combined with other seed treatments such as fungicides, insecticides, and bio-enhancers.

In one embodiment, said treating involves a compound of formula (I) (or other compound(s) described herein) that is a stimulant to a nematode. As used herein, the term “stimulant” means a compound that promotes the growth and/or development of nematodes or nematode eggs or larvae.

In one embodiment, said contacting is carried out simultaneously or nearly simultaneously with planting a plant other than a plant vulnerable to a nematode. According to this embodiment, a compound of formula (I) (or other compound(s) described herein) may be effective in treating a nematode by promoting nematode growth and/or development in the absence of a critical plant host, which results in the inability of the nematode to grow, reproduce, hatch, or survive (death from starvation), thus reducing the presence of or eliminating the nematode from growing media to permit successful cultivation of plants vulnerable to a nematode in the treated growing media.

In carrying out the methods disclosed herein, contacting may be carried out by any suitable means, including those common in agricultural settings for application of chemicals to plants and/or growing media. Such methods include, without limitation, application to a plant, a growing media, soil, or planting area by high or low pressure spraying. Suitable application means may also include atomizing, foaming, fogging, coating, and encrusting. Contacting may be carried out using any formulation of the compounds described herein, including formulations of the compositions described infra.

A further aspect of the present application relates to a composition comprising a compound of formula (I) (or other compound(s) described herein) and an agriculturally acceptable carrier.

According to one embodiment, the composition is formulated into any suitable form including, without limitation, a solution, emulsion, emulsifiable concentrate, suspension, foam, paste, aerosol, suspoemulsion concentrate, slurry, or dry powder. Suitable compositions may include, for example and without limitation, those for HV, LV, and ULV spraying and for ULV cool and warm fogging formulations. In one particular embodiment, the composition is formulated in a manner suitable for large or small scale agricultural and horticultural applications.

Compositions may be produced in a known manner, for example, by mixing a liquid composition with extenders, that is, liquid solvents, liquefied gases under pressure, and/or solid carriers. Wetting agents and/or surfactants, that is, emulsifiers and/or dispersants, sequestering agents, plasticizers, brighteners, flow agents, coalescing agents, waxes, fillers, polymers, anti-freezing agents, biocides, thickeners, tackifiers, and/or foam formers and defoaming agents may also be used in manners commonly known by those of ordinary skill in the art. If the extender used is water, it is also possible to employ, for example, organic solvents as auxiliary solvents. Other possible additives are mineral and vegetable oils, colorants such as inorganic pigments, and trace nutrients.

The nature and action of such additives are well-known to those of ordinary skill in the art of liquid formulations. Additives should not interfere with the action of the nematacide compound or any other biologically active component that may be included in the formulation.

The active compound(s) content of the composition (e.g., one or more compounds as described herein) may vary within a wide range. For example, the concentration of active compound (i.e., one or more compounds described herein) may be from 0.0000001 to 20% by weight, or from 0.0001 to 15% by weight.

In one embodiment, it may be desirable to combine the composition of the present application with effective amounts of other agricultural or horticultural chemicals, such as herbicides (e.g., glyphosate), insecticides, acaracides, other nematicides, molluscicides, attractants, sterilants, bactericides, fungicides, and/or growth regulators.

One common herbicide is glyphosate, commonly known as 2 (phosphonomethylamino)acetic acid. Glyphosate salts may also be used. Suitable glyphosate salts include, for example, but are not limited to, isopropylamine salts, diammonium salts, and trimethylsulfonium salts. Mixtures including glyphosate typically include one or more surfactants, typically one or more nonionic surfactants, though no surfactant should be required. Glyphosate-containing formulations are typically applied to desirable plants and plant-parts that are glyphosate resistant.

Examples of other herbicides that may be useful in compositions described herein include, for example, but are not limited to: amide herbicides, including allidochlor, amicarbazone, beflubutamid, benzadox, benzipram, bromobutide, cafenstrole, CDEA, cyprazole, dimethenamid, dimethenamid-P, diphenamid, epronaz, etnipromid, fentrazamide, flucarbazone, flupoxam, fomesafen, halosafen, isocarbamid, isoxaben, napropamide, naptalam, pethoxamid, propyzamide, quinonamid, saflufenacil, and tebutam; anilide herbicides, including chloranocryl, cisanilide, clomeprop, cypromid, diflufenican, etobenzanid, fenasulam, flufenacet, flufenican, ipfencarbazone, mefenacet mefluidide, metamifop, monalide, naproanilide, pentanochlor, picolinafen, propanil, sulfentrazone; arylalanine herbicides, including benzoylprop, flamprop, and flamprop-M; chloroacetanilide herbicides, including acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor, and xylachlor; sulfonanilide herbicides, including benzofluor, cloransulam, diclosulam, florasulam, flumetsulam, metosulam, perfluidone, pyrimisulfan, and profluazol; sulfonamide herbicides, including asulam, carbasulam, fenasulam, oryzalin, penoxsulam, and pyroxsulam; thioamide herbicides, including bencarbazone and chlorthiamid; antibiotic herbicides, including bilanafos; aromatic acid herbicides; benzoic acid herbicides, including chloramben, dicamba, 2,3,6-TBA, and tricamba; pyrimidinyloxybenzoic acid herbicides, including bispyribac and pyriminobac; pyrimidinylthiobenzoic acid herbicides, including pyrithiobac; phthalic acid herbicides, including chlorthal, picolinic acid herbicides, aminopyralid, clopyralid, and picloram; quinolinecarboxylic acid herbicides, including quinclorac and quinmerac; arsenical herbicides, including cacodylic acid, CMA, DSMA, hexaflurate, MAA, MAMA, MSMA, potassium arsenite, and sodium arsenite; benzoylcyclohexanedione herbicides, including mesotrione, sulcotrione, tefuryltrione, and tembotrione; benzofuranyl alkylsulfonate herbicides, including benfuresate and ethofumesate; benzothiazole herbicides, including benazolin, benzthiazuron, fenthiaprop, mefenacet, and methabenzthiazuron; carbamate herbicides, including asulam, carboxazole, chlorprocarb, dichlormate, fenasulam, karbutilate, and terbucarb; carbanilate herbicides, including barban, BCPC, carbasulam, carbetamide, CEPC, chlorbufam, chlorpropham, CPPC, desmedipham, phenisopham, phenmedipham, phenmedipham-ethyl, propham, and swep; cyclohexene oxime herbicides, including alloxydim, butroxydim, clethodim, cloproxydim, cycloxydim, profoxydim, sethoxydim, tepraloxydim, and tralkoxydim; cyclopropylisoxazole herbicides, including isoxachlortole and isoxaflutole; dicarboximide herbicides, including cinidon-ethyl, flumezin, flumiclorac, flumioxazin, and flumipropyn; dinitroaniline herbicides, including benfluralin, butralin, dinitramine, ethalfluralin, fluchloralin, isopropalin, methalpropalin, nitralin, oryzalin, pendimethalin, prodiamine, profluralin, and trifluralin; dinitrophenol herbicides, including dinofenate, dinoprop, dinosam, dinoseb, dinoterb, DNOC, etinofen, and medinoterb; diphenyl ether herbicides, including ethoxyfen; nitrophenyl ether herbicides, including acifluorfen, aclonifen, bifenox, chlomethoxyfen, chlornitrofen, etnipromid, fluorodifen, fluoroglycofen, fluoronitrofen, fomesafen, furyloxyfen, halosafen, lactofen, nitrofen, nitrofluorfen, and oxyfluorfen; dithiocarbamate herbicides, including dazomet and metam; halogenated aliphatic herbicides, including alorac, chloropon, dalapon, flupropanate, hexachloroacetone, iodomethane, methyl bromide, monochloroacetic acid, SMA, and TCA; imidazolinone herbicides, including imazamethabenz, imazamox, imazapic, imazapyr, imazaquin, and imazethapyr; inorganic herbicides, including ammonium sulfamate, borax, calcium chlorate, copper sulfate ferrous sulfate, potassium azide, potassium cyanate, sodium azide, sodium chlorate, and sulfuric acid; nitrile herbicides, including bromobonil, bromoxynil, chloroxynil, dichlobenil, iodobonil, ioxynil, and pyraclonil; organophosphorus herbicides, including amiprofos-methyl, anilofos, bensulide, bilanafos, butamifos, 2,4-DEP, DMPA, EBEP, fosamine, glufosinate, glufosinate-P, glyphosate, and piperophos; oxadiazolone herbicides, including dimefuron, methazole, oxadiargyl, and oxadiazon; oxazole herbicides, including carboxazole, isouron, isoxaben, isoxachlortole, isoxaflutole, monisouron, pyroxasulfone, and topramezone; phenoxy herbicides, including bromofenoxim, clomeprop, 2,4-DEB, 2,4-DEP, difenopenten, disul, erbon, etnipromid, fenteracol, and trifopsime; phenoxyacetic herbicides, including 4-CPA, 2,4-D, 3,4-DA, MCPA, MCPA-thioethyl, and 2,4,5-T; phenoxybutyric herbicides, including 4-CPB, 2,4-DB, 3,4-DB, MCPB, and 2,4,5-TB; phenoxypropionic herbicides, including cloprop, 4-CPP, dichlorprop, dichlorprop-P, 3,4-DP, fenoprop, mecoprop, and mecoprop-P; aryloxyphenoxypropionic herbicides, including chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P, and trifop; phenylenediamine herbicides, including dinitramine, and prodiamine; pyrazole herbicides, including azimsulfuron, difenzoquat, halosulfuron, metazachlor, pyrazosulfuron, and pyroxasulfone; benzoylpyrazole herbicides, including benzofenap, pyrasulfotole, pyrazolynate, pyrazoxyfen, and topramezone; phenylpyrazole herbicides, including fluazolate, nipyraclofen, and pyraflufen; pyridazine herbicides, including credazine, pyridafol, and pyridate; pyridazinone herbicides, including brompyrazon, chloridazon, dimidazon, flufenpyr, metflurazon, norflurazon, oxapyrazon, and pydanon; pyridine herbicides, including aminopyralid, cliodinate, clopyralid, diflufenican, dithiopyr, flufenican, fluroxypyr, haloxydine, picloram, picolinafen, pyriclor, pyroxsulam, thiazopyr, and triclopyr; pyrimidinediamine herbicides, including iprymidam and tioclorim; quaternary ammonium herbicides, including cyperquat, diethamquat, difenzoquat, diquat, morfamquat, and paraquat; thiocarbamate herbicides, including butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate, and vernolate; thiocarbonate herbicides, including dimexano, EXD, and proxan; thiourea herbicides, including methiuron; triazine herbicides, including dipropetryn, triaziflam, and trihydroxytriazine; chlorotriazine herbicides, including atrazine, chlorazine, cyanazine, cyprazine, eglinazine, ipazine, mesoprazine, procyazine, proglinazine, propazine, sebuthylazine, simazine, terbuthylazine, and trietazine; methoxytriazine herbicides, including atraton, methometon, prometon, secbumeton, simeton, and terbumeton; methylthiotriazine herbicides, including ametryn, aziprotryne, cyanatryn, desmetryn, dimethametryn, methoprotryne, prometryn, simetryn, and terbutryn; triazinone herbicides, including ametridione, amibuzin, hexazinone, isomethiozin, metamitron, and metribuzin; triazole herbicides, including amitrole, cafenstrole, epronaz, and flupoxam; triazolone herbicides, including amicarbazone, bencarbazone, carfentrazone, flucarbazone, ipfencarbazone, propoxycarbazone, sulfentrazone, and thiencarbazone; triazolopyrimidine herbicides, including cloransulam, diclosulam, florasulam, flumetsulam, metosulam, penoxsulam, and pyroxsulam; uracil herbicides, including benzfendizone, bromacil, butafenacil, flupropacil, isocil, lenacil, saflufenacil, and terbacil; urea herbicides, including benzthiazuron, cumyluron, cycluron, dichloralurea, diflufenzopyr, isonoruron, isouron, methabenzthiazuron, monisouron, and noruron; phenylurea herbicides, including anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluron, phenobenzuron, siduron, tetrafluron, and thidiazuron; sulfonylurea herbicides; pyrimidinylsulfonylurea herbicides, including amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulfuron, pyrazosulfuron, rimsulfuron, sulfometuron, sulfosulfuron, and trifloxysulfuron; triazinylsulfonylurea herbicides, including chlorsulfuron, cinosulfuron, ethametsulfuron, iodosulfuron, metsulfuron, prosulfuron, thifensulfuron, triasulfuron, tribenuron, triflusulfuron, and tritosulfuron; thiadiazolylurea herbicides, including buthiuron, ethidimuron, tebuthiuron, thiazafluron, and thidiazuron; and unclassified herbicides, including acrolein, allyl alcohol, aminocyclopyrachlor, azafenidin, bentazone, benzobicyclon, buthidazole, calcium cyanamide, cambendichlor, chlorfenac, chlorfenprop, chlorflurazole, chlorflurenol, cinmethylin, clomazone, CPMF, cresol, cyanamide, ortho-dichlorobenzene, dimepiperate, endothal, fluoromidine, fluridone, flurochloridone, flurtamone, fluthiacet, indanofan, methyl isothiocyanate, OCH, oxaziclomefone, pentachlorophenol, pentoxazone, phenylmercury acetate, pinoxaden, prosulfalin, pyribenzoxim, pyriftalid, quinoclamine, rhodethanil, sulglycapin, thidiazimin, tridiphane, trimeturon, tripropindan, and tritac. The above list is exemplary only and other herbicides may also be used in conjunction with the compositions disclosed herein.

Examples of specific insecticides, acaracides, nematicides, and molluscicides that may be used in compositions taught herein include, but are not limited to: abamectin, acephate, acetamiprid, acrinathhn, alanycarb, aldicarb, alpha-cypermethrin, alphamethrin, amitraz, azinphos A, azinphos-methyl, azocyclotin, bendiocarb, benfuracarb, bensultap, beta cyfluthrin, bifenthrin, brofenprox, bromophos A, bufencarb, buprofezin, butocarboxin, butylpyridaben, cadusafos, carbaryl, carbofuran, carbophenothion, carbosulfan, cartap, chloethocarb, chloranthraniliprole, chloroethoxyfos, chlorfenvenphos, chlorofluazuron, chloromephos, chloropyrifos, cis-res-methrin, clocythrin, clofentezin, clothianidin, cyanoimine, cyanophos, cycloprothhn, cyfluthrin, cyhexatin, deltamethrin, demeton M, demeton S, demeton-S-methyl, diafenthiuron, dibutylaminothio, dichlofenthion, dicliphos, diethion, diflubenzuron, dimethoate, dimethylvinphos, dinotefuran, dioxathion, doramectin, edifenphos, emamectin, endosulfan, esfenvalerate, ethiofencarb, ethion, ethiprole, ethofenprox, ethoprophos, etrimphos, fenamiphos, fenazaquin, fenbutatin oxide, fenitrothion, fenobucarb, fenothiocarb, fenoxycarb, fenpropathrin, fenpyrad, fenpyroximate, fenthion, fenvalerate, fipronil, fluazinam, flubendiamide, flucycloxuron, flucythrinate, flufenoxuron, flufenprox, fluxofenime, fonophos, formothion, fosthiazate, fubfenprox, gamma cyhalothrin, HCH, heptenophos, hexaflumuron, hexythiazox, imidacloprid, iprobenfos, isoprocarb, isoxathion, ivermectin, lambda cyhalothrin, lindane, lufenuron, malathion, mecarbam, mesulfenphos, metaldehyde, methamidophos, methiocarb, methomyl, metolcarb, mevinphos, milbemectin, milbemycin oxime, moxidectin, naled, NC 184, nitenpyram, nitromethylene, omethoate, oxamyl, oxydemethon M, oxydeprofos, parathion, parathion-methyl, permethrin, phenthoate, phorate, phosalone, phosmet, phoxim, pirimicarb, pirimiphos A, pirimiphos M, promecarb, propaphos, propoxur, prothiofos, prothoate, pymetrozine, pyrachlophos, pyrada-phenthion, pyresmethrin, pyrethrum, pyridaben, pyrimidifen, pyripfoxyfen, pyriproxyfen, rynaxypyr, salithion, sebufos, silafluofen, sulfotep, sulprofos, tebufenozide, tebufenpyrad, tebupihmphos, teflubenzuron, tefluthrin, temephos, terbam, terbufos, tetrachloro-vinphos, thiacloprid, thiafenox, thiamethoxam, thiodicarb, thiofanox, thionazin, thuringiensin, tralomethrin, triarthen, triazamate, triazophos, triazuron, trichlorofon, triflumuron, trimethacarb, vamidothion, xylylcarb, zeta-cypermethrin, zetamethrin, and Bacillus thuringiensis (Bt) products, including the salts and esters thereof. The above list is exemplary only and other insecticides may also be used in conjunction with the compositions disclosed herein.

A variety of fungicides may be used in embodiments of the compositions disclosed herein. They include, for example and without limitation, those classified and listed by the Fungicide Resistance Action Committee (FRAC), FRAC CODE LIST 1: Fungicides sorted by FRAC Code, December 2006, which is hereby incorporated by reference in its entirety. A summary of this list includes: Methyl benzimidazole carbamates (MBC): e.g., benzimidazoles and thiophanates; Dicarboximides; Demethylation inhibitors (DMI) (SBI: Class I): e.g., imidazoles, piperazines, pyridines, pyrimidines, and triazoles; Phenylamides (PA): e.g., acylalanines, oxazolidinones, and butyrolactones; Amines (SBI: Class II): e.g., morpholines, piperidines, and spiroketalamines; Phosphoro-thiolates and Dithiolanes; Carboxamides: e.g., benzamides, furan carboxamides, oxathiin carboxamides, thiazole carboxamides, pyrazole carboxamides, and pyridine carboxamides; Hydroxy-(2-amino-) pyrimidines; Anilino-pyrimidines (AP); N-phenyl carbamates; Quinone outside inhibitors (QoI): e.g., methoxyacrylates, methoxy-carbamates, oximino acetates, oximino-acetamides, oxazolidine-diones, dihydro-dioxazines, imidazolinones, and benzyl-carbamates; Phenylpyrroles; Quinolines; Aromatic hydrocarbons (AH) and Heteroaromatics I: e.g., 1,2,4-thiadiazoles; Cinnamic acids; Melanin biosynthesis inhibitors-reductase (MBI-R): e.g., isobenzofuranone, pyrroloquinolinone, and triazolobenzo-thiazole; Melanin biosynthesis inhibitors-dehydratase (MBI-D): e.g., cyclopropane-carboxamide, carboxamide, and propionamide; Hydroxyanilides (SBI: Class III); Hydroxyanilides (SBI: Class IV): e.g., thiocarbamates and allylamines; Polyoxins: e.g., peptidyl pyrimidine nucleoside; Phenylureas; Quinone inside inhibitors (QiI): e.g., cyanoimidazole and sulfamoyl-triazoles; Benzamides: e.g., toluamides; Antibiotics: e.g., enopyranuronic acid, hexopyranosyl, streptomycin, and validamycin; Cyanoacetamide-oximes; Carbamates; Dinitrophenyl crotonates; Pyrimidinone-hydrazones; 2,6-dinitro-anilines; Organo tin compounds: e.g., tri phenyl tin compounds; Carboxylic acids; Heteroaromatics II: e.g., isoxazoles and isothiazolones; Phosphonates: e.g., ethyl phosphonates and phosphorous acid and salts; Phthalamic acids; Benzotriazines; Benzene-sulfonamides; Pyridazinones; Thiophene-carboxamides; Pyrimidinamides; CAA-fungicides (Carboxylic Acid Amides): e.g., cinnamic acid amides, valinamide carbamates and mandelic acid amides; Tetracycline; Thiocarbamate; Benzamides: e.g., acylpicolides; Host plant defense inducers: e.g., benzo-thiadiazole BTH, benzisothiazole and thiadiazole-carboxamides; Unclassified materials: e.g., thiazole carboxamide, phenyl-acetamide, quinazolinone, and benzophenone; Multi-site contact materials: e.g., copper salts, sulfur, dithiocarbamates and relatives, phthalimides, chloronitriles (phthalonitriles), sulphamides, guanidines, triazines, and quinones (anthraquinones); Non-classified materials: e.g., mineral oils, organic oils, potassium bicarbonate, and biological materials. Those skilled in the art will recognize that use of other fungicides is also possible in various embodiments of the application.

This and other compositions disclosed herein may contain additional additives, such as a fertilizer.

Compositions contemplated herein may be microencapsulated in a polymeric substance. Examples of suitable microencapsulation materials include the following classes of materials for which representative members are provided. It will be apparent to those skilled in the art that other classes of materials with polymeric properties may be used and that other materials within each given class and others polymeric classes may be used for microencapsulation. In this description, microencapsulation is taken to include methods and materials for nanoencapsulation. Examples include but are not limited to: gums and natural macromolecules, such as gum arabic, agar, sodium alginate, carageenan, and gelatin; carbohydrates, such as starch, dextran, sucrose, corn syrup, and β-cyclodextrin; celluloses and semisynthetic macromolecules, such as carboxymethylcellulose, methycellulose, ethylcellulose, nitrocellulose, acetylcellulose, cellulose acetate-phthalate, cellulose acetate-butylate-phthalate, epoxy, and polyester; lipids such as wax, paraffin, stearic acid, monoglycerides, phospholipids, diglycerides, beeswax, oils, fats, hardened oils, and lecithin; inorganic materials, such as calcium sulfate, silicates, and clays; proteins, such as gluten, caseine, gelatine, and albumine; biological materials, such as voided cells from organisms like baker's yeast and other microorganisms together with other formerly living cell tissues. Furthermore, these materials may be used singly or compounded in the processes of micro- or nano-encapsulation.

In one embodiment, one or more compounds described herein can be applied to plant seeds (e.g., as a seed coating) with other conventional seed formulation and treatment materials including, without limitation, clays and polysaccharides.

Compositions disclosed herein may be applied, e.g., to a plant, a growing media, soil, or planting area, by high or low pressure spraying. Suitable application means may also include atomizing, foaming, fogging, coating, and encrusting.

When treating plant seeds, the composition can be applied by low or high pressure spraying, coating, or immersion. Other suitable application procedures can be envisioned by those skilled in the art. Once soil, growing medium, or a plant seed is treated with the composition, seeds can be planted and cultivated using conventional procedures to produce plants. After plants have been propagated from seeds, soil, or growing medium treated with compositions disclosed herein, the soil or growing medium may be treated with one or more applications of the composition described herein to impart disease resistance to plants, to enhance plant growth, to control disease on the plants, and/or impart stress resistance.

In one embodiment, application of the composition is to soil or a growing medium for plants vulnerable to nematode disease. Applying the composition to a soil or a growing medium may be carried out at a rate of about 0.1 to 10,000 g/ha of a composition disclosed herein.

In another embodiment, application of the composition is to plant seed. Applying the composition to a plant seed may be carried out at a rate of about 0.001 to 50 g/kg of the composition to seed.

Another aspect of the present application relates to a compound of formula (II) having the following structure:

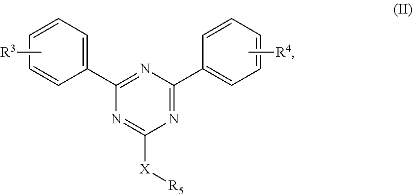

or a stereoisomer, salt, oxide, or solvate thereof, wherein

R³ and R⁴ are independently present or absent, and when present are independently halogen, alkoxy, or C₁-C₆ alkyl;

X is O or S; and

R⁵ is C₁-C₆ alkyl.

A further aspect of the present application relates to a compound of formula (III) having the following structure:

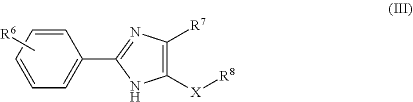

or a stereoisomer, salt, oxide, or solvate thereof, wherein

R⁶ is present or absent and when present is a halogen, alkoxy, or C₁-C₆ alkyl;

R⁷ is selected from H, C₁-C₆ alkyl, alkoxy, and —COO(CH₂)ₙCH₃;

X is S or O;

R⁸ is —(CH₂)ₙCOO(CH₂)nCH₃; and

n is an integer between 0-3.

Another aspect of the present application relates to a method of treating a plant or a growing media for a nematode. This method involves contacting a plant or a growing media with any one or more compounds described herein, such as a compound of formula (II) or formula (III), to treat the plant or growing media for a nematode.

A further aspect of the present application relates to a composition comprising a compound of formula (II) or formula (III) as described herein and an agriculturally acceptable carrier.

A further aspect of the present application relates to a method of making a compound formula (I) having the following structure:

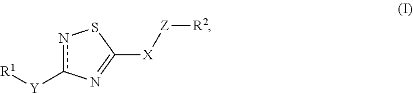

or a stereoisomer, salt, oxide, or solvate thereof, wherein

Y is a bond; —(CH₂)ₙ—; or —CH═;

R¹ is phenyl optionally substituted one or more times with halogen, CF₃, alkoxy, C₁-C₆ alkyl, NO₂, —OCF₃, —OCF₂H, —CN, alkylamine, or alkylthio; pyridine; pyrazine; carboxyalkyl; —CONH₂: alkoxy; alkylsulfide; alkylamine; benzylpiperazine; H; CF₃; and O- and N^{α}-protected amino acids comprising a carboxylate protected by a first protecting group and an amino group protected by a second protecting group; and

X is O or S;

Z is —(CH₂)ₙ—;

R² is selected from the group consisting of C₁-C₆ alkyl optionally substituted one or more times with halogen, alkoxy, or NH with an amine protecting group; C₂-C₁₀ alkenyl; phenyl optionally substituted one or more times with halogen or alkoxy; —COO(CH₂)ₙCH₃; —CO(CH₂)ₙCH₃; and an alcohol; and

n is an integer selected from 0-3. This method involves providing a starting material comprising an amidine, isourea, guanidine, or isothiourea molecule; reacting the starting material with carbon disulfide or an alkyl imidazole-1-carbodithioate molecule to form a dithiocarbamate compound; and converting the dithiocarbamate compound to a compound of formula (I).

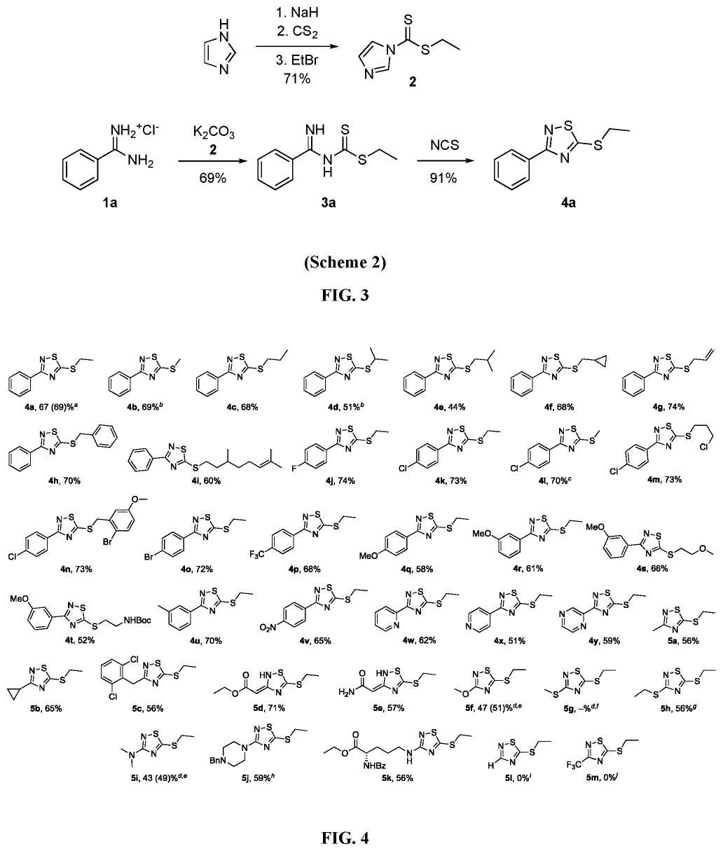

The method of making a compound of formula (I) of the present application is novel in allowing 1,2,4-thiadiazoles to be synthesized in one pot using an amidine, isourea, guanidine, or isothiourea; with carbon disulfide or an alkyl imidazole-1-carbodithioate (FIG. 3 and Table 1); in the presence of a suitable base, such as DBU, DBN, potassium carbonate, potassium phosphate, or sodium bicarbonate; in a suitable polar, aprotic solvent such as acetonitrile, tetrahydrofuran, DMF, sulfolane, DMPU, HMPA, with a suitable oxidizing agent, including NCS, NBS, NIS, potassium peroxymonosulfate, mCPBA, iodine, hypervalent iodine compounds like phenyliodine(III) diacetate and iodosobenzene bis(trifluoroacetate); trichloroisocyuranic acid, 1,3-dibromo-5,5-dimethylhydantoin, hydrogen peroxide, catalytic copper/air, etc.

In one embodiment, the method of making described herein further requires either the appropriate alkyl halide (e.g., methyl iodide, ethyl bromide, ethyl iodide, isopropyl iodide, allyl bromide, allyl chloride, benzyl chloride, 2-methoxyethyl bromide) or a Michael acceptor (e.g., acrolein, methyl acrylate, methyl acrylate, acrylamide, 4-acryloylmorpholine, acrylonitrile, methyl vinyl ketone, N,N-dimethylacrylamide, phenyl vinyl sulfide, phenyl vinyl sulfone, ethenesulfonyl fluoride) or an epoxide (e.g. 1,2-epoxybutane, ethylene oxide).

In one embodiment, the method involves making a compound of formula (I) where X is O. Such compounds may be made as described supra, but by replacing the solvent entirely with the appropriate alcohol, or using the appropriate alcohol in a mix with an aprotic solvent, especially acetonitrile.

Ketene Dithioacetals

Another aspect of the present application relates to a compound of formula (IV) having the following structure:

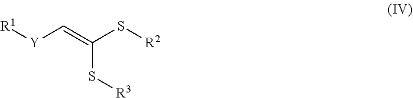

or a stereoisomer, salt, oxide, or solvate thereof, wherein

Y is a bond or C₂-C₁₀ alkylene;

R¹ is optional, and when present is phenyl optionally substituted at one or more positions with halogen, CF₃, NO₂, —CN, C₁-C₆ alkyl, C₁-C₆ alkoxy optionally substituted one or more times with halogen, C₁-C₆ alkylthio, —SCF₃, C₁-C₆ alkylamine; benzodioxolyl optionally substituted one or more times with halogen; naphthalene; thiophene; indole optionally substituted one or more times with C₁-C₆ alkyl; and pyridine optionally substituted one or more times with halogen; and

R² and R³ are independently selected from C₁-C₈ alkyl and phenyl,

with the following provisos:

when Y is a bond and R¹ is phenyl or phenyl substituted one time with a halogen, NO₂ or MeO, R² and R³ are not ethyl;

when Y is a bond and R¹ is naphthalene, R² and R³ are not methyl; and

when Y is C₂ alkylene and R¹ is phenyl, R² and R³ are not ethyl.

In one embodiment of the compound of formula (IV) Y is a bond.

In one embodiment of the compound of formula (IV) R¹ is phenyl.

In one embodiment of the compound of formula (IV) R¹ is phenyl substituted with halogen.

In one embodiment of the compound of formula (IV) R¹ is phenyl substituted with CF₃.

In one embodiment of the compound of formula (IV) R¹ is phenyl substituted with NO₂.

In one embodiment of the compound of formula (IV) R¹ is phenyl substituted with —CN.

In one embodiment of the compound of formula (IV) R¹ is phenyl substituted with C₁-C₆ alkyl.

In one embodiment of the compound of formula (IV) R¹ is phenyl substituted with C₁-C₆ alkoxy optionally substituted one or more times with halogen.

In one embodiment of the compound of formula (IV) R¹ is phenyl substituted with —OCF₃ or —OCF₂H.

In one embodiment of the compound of formula (IV) R¹ is phenyl substituted with C₁-C₆ alkylthio.

In one embodiment of the compound of formula (IV) R¹ is phenyl substituted with —SCF₃.

In one embodiment of the compound of formula (IV) R¹ is phenyl substituted with C₁-C₆ alkylamine.

In one embodiment of the compound of formula (IV) R¹ is benzodioxolyl optionally substituted one or more times with halogen.

In one embodiment of the compound of formula (IV) R¹ is thiophene.

In one embodiment of the compound of formula (IV) R¹ is indole optionally substituted one or more times with C₁-C₆ alkyl.

In one embodiment of the compound of formula (IV) R¹ is pyridine optionally substituted one or more times with halogen.

In one embodiment, the compound of formula (IV) has the following structure:

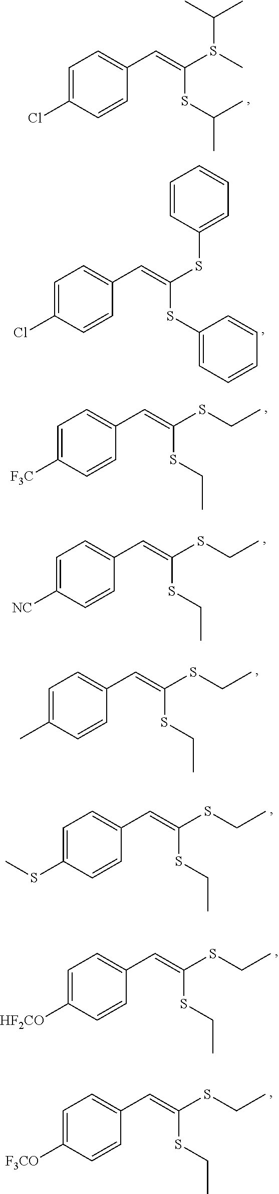

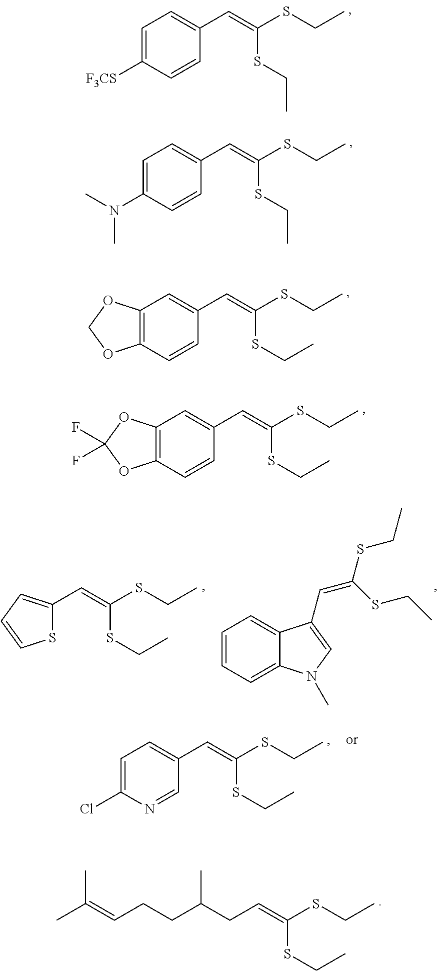

Another aspect of the present application relates to a method of treating a plant or growing media for a nematode. This method involves contacting a plant or growing media with a compound of formula (IV) having the following structure:

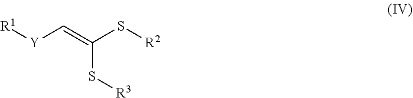

or a stereoisomer, salt, oxide, or solvate thereof, wherein

Y is a bond or C₂-C₁₀ alkylene;

R¹ is optional, and when present is phenyl optionally substituted at one or more positions with halogen, CF₃, NO₂, —CN, C₁-C₆ alkyl, C₁-C₆ alkoxy optionally substituted one or more times with halogen, C₁-C₆ alkylthio, —SCF₃, C₁-C₆ alkylamine; benzodioxolyl optionally substituted one or more times with halogen; naphthalene; thiophene; indole optionally substituted one or more times with C₁-C₆ alkyl; and pyridine optionally substituted one or more times with halogen; and

R² and R³ are independently selected from C₁-C₈ alkyl and phenyl to treat the plant or growing media for a nematode.

A further aspect of the present application relates to a method of treating a plant or growing media for a nematode. This method involves contacting a plant or growing media with a compound of formula (V) having the following structure:

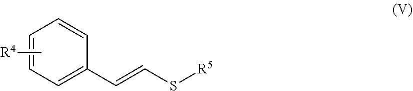

or a stereoisomer, salt, oxide, or solvate thereof, wherein

R⁴ is selected from C₁-C₆ alkyl, alkoxy, CF₃, and halogen and R⁵ is C₁-C₆ alkyl to treat the plant or growing media for a nematode.

According to aspects of the present application, a plant or a growing media is treated with a compound of formula (IV) and/or formula (V).

In one embodiment, a plant is treated with a compound of formula (IV) and/or formula (V).

Suitable plants amenable to the treatment methods described herein include any plant vulnerable or susceptible to nematodes. A number of genera and species of nematodes are known to be highly damaging to a great number of plant hosts, including foliage plants, agronomic and vegetable crops, fruit and nut trees, turfgrass, and forest trees.

In one embodiment, a plant treated with a compound of formula (IV) and/or formula (V) is a vegetable crop. In a particular embodiment, the plant is soybean (Glycine max).

Some of the most damaging nematodes to plants include, without limitation, root-knot (Meloidogyne spp.); cyst (Heterodera and Globodera spp.); root-lesion (Pratylenchus spp.); spiral (Helicotylenchus spp.); burrowing (Radopholus similis); bulb and stem (Ditylenchus dipsaci); reniform (Rotylenchulus reniformis); dagger (Xiphinema spp.); bud and leaf (Aphelenchoides spp.); and Pine Wilt Disease (Bursaphelenchus xylophilus). According to the present disclosure, any of these (or any other plant-parasitic nematode) is treated according to the methods described herein.

In one embodiment, a growing media is treated with a compound of formula (IV) and/or formula (V).

In carrying out methods described herein, a plant or a growing media is contacted with a compound of formula (IV) and/or formula (V).

Contacting a plant or a growing media with a compound of formula (IV) and/or formula (V) may involve contacting a plant or growing media with a compound of formula (IV) and/or formula (V) or a composition disclosed herein, which composition contains a compound of formula (IV) and/or formula (V).

Compounds of formula (IV) suitable in the methods of the present application have substituents as defined supra.

In one embodiment, the compound of formula (IV) has the following structure:

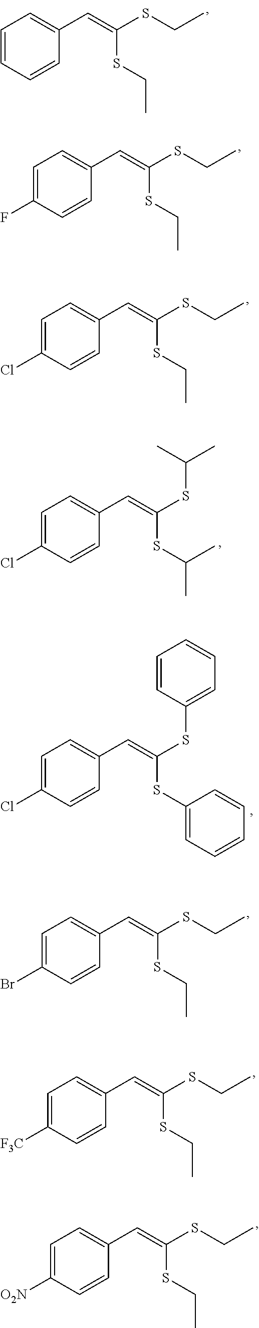

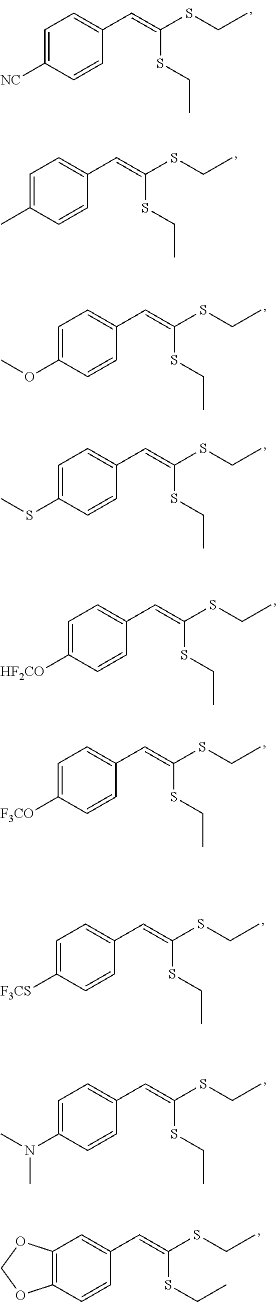

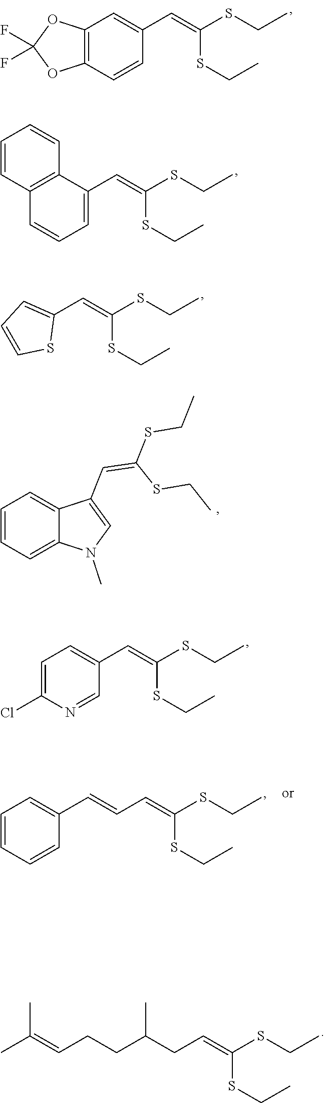

In one embodiment the method involves a compound of formula (V) having the following structure

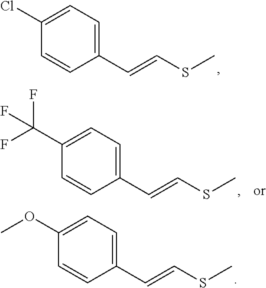

In one embodiment of carrying out said contacting, the compound of formula (IV) and formula (V) is a nematacide as described herein.

In one embodiment, said contacting is carried out simultaneously or nearly simultaneously with planting seed in a growing media. In other words, according to one embodiment, the method is carried out simultaneously with planting a seed vulnerable (at the seed or, more likely, the plant stage) to a nematode. According to this embodiment, treatment of a growing media may happen at or near the time the seed is planted in the growing media. Alternatively, treatment of the growing media with the compound of formula (IV) and/or formula (V) may occur via a pre-treated seed (e.g., a coating on the seed that contains a compound of formula (IV) and/or formula (V), which comes into contact with the growing media to be treated at the time of planting the seed in the growing media). Seed treatment with the compound of formula (IV) and/or formula (V) can be combined with other seed treatments such as fungicides, insecticides, and bio-enhancers.

In one embodiment, said treating involves a compound of formula (IV) and/or formula (V) that is a stimulant to a nematode. As used herein, the term “stimulant” means a compound that promotes the growth and/or development of nematodes or nematode eggs or larvae.

In one embodiment, said contacting is carried out simultaneously or nearly simultaneously with planting a plant other than a plant vulnerable to a nematode. According to this embodiment, a compound of formula (IV) and/or formula (V) may be effective in treating a nematode by promoting nematode growth and/or development in the absence of a critical plant host, which results in the inability of the nematode to grow, reproduce, hatch, or survive (death from starvation), thus reducing the presence of or eliminating the nematode from growing media to permit successful cultivation of plants vulnerable to a nematode in the treated growing media.

In carrying out the methods disclosed herein, contacting may be carried out by any suitable means, as described herein.

Another aspect of the present application relates to a method of forming a ketene dithioacetal compound having a structure of formula (VI)

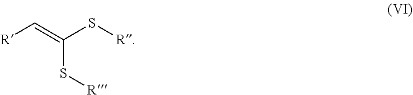

This method involves providing a dimethyl methyl phosphonate compound having a structure of

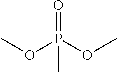

and
reacting the dimethyl methyl phosphonate compound with a disulfide compound and an aldehyde to produce the compound of formula (VI), wherein R′, R″, and R′″ are any compatible substituent.

In one embodiment, the method of this aspect of the application is carried out in a single reaction container.

In another embodiment, reacting comprises combining the dimethyl methyl phosphonate with the disulfide in the presence of an amide base.

In one embodiment, the amide base is selected from the group consisting of lithium diisopropylamide (LDA), lithium tetramethylpiperidine (LiTMP), and sodium or potassium analogs.

In one embodiment, the disulfide compound is selected from ethyl disulfide, dimethyl disulfide, diisopropyl disulfide, and diphenyl disulfide.

In one embodiment, reacting the dimethyl methyl phosphonate compound with a disulfide compound produces a compound having a structure of formula (VII), as follows:

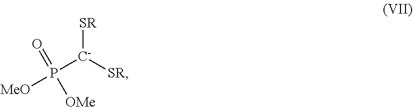

wherein R is any compatible substituent.

In one embodiment, the compound of formula (VII) is reacted with an aldehyde to produce the compound of formula (VI).

A further aspect of the present application relates to a composition comprising a compound of formula (IV) (or any other compound described herein) and an agriculturally acceptable carrier.

According to one embodiment, the composition is formulated into any suitable form including, without limitation, a solution, emulsion, emulsifiable concentrate, suspension, foam, paste, aerosol, suspoemulsion concentrate, slurry, or dry powder. Suitable compositions may include, for example and without limitation, those for HV, LV, and ULV spraying and for ULV cool and warm fogging formulations. In one particular embodiment, the composition is formulated in a manner suitable for large or small scale agricultural and horticultural applications.

Compositions may be produced in a known manner, for example, by mixing a liquid composition with extenders, that is, liquid solvents, liquefied gases under pressure, and/or solid carriers. Wetting agents and/or surfactants, that is, emulsifiers and/or dispersants, sequestering agents, plasticizers, brighteners, flow agents, coalescing agents, waxes, fillers, polymers, anti-freezing agents, biocides, thickeners, tackifiers, and/or foam formers and defoaming agents may also be used in manners commonly known by those of ordinary skill in the art. If the extender used is water, it is also possible to employ, for example, organic solvents as auxiliary solvents. Other possible additives are mineral and vegetable oils, colorants such as inorganic pigments, and trace nutrients.

The nature and action of such additives are well-known to those of ordinary skill in the art of liquid formulations. Additives should not interfere with the action of a compound of formula (IV) or any other biologically active component that may be included in the formulation.

The active compound(s) content of the composition (e.g., one or more compounds as described herein) may vary within a wide range, as described herein.

In one embodiment, it may be desirable to combine the composition of the present disclosure with effective amounts of other agricultural or horticultural chemicals, such as herbicides (e.g., glyphosate), insecticides, acaracides, other nematicides, molluscicides, attractants, sterilants, bactericides, fungicides, and/or growth regulators as described herein.

The composition disclosed herein may contain additional additives, such as a fertilizer.

Compositions contemplated herein may be microencapsulated in a polymeric substance as described herein.

In one embodiment, one or more compounds described herein can be applied to plant seeds (e.g., as a seed coating) with other conventional seed formulation and treatment materials including, without limitation, clays and polysaccharides.

Compositions disclosed herein may be applied, e.g., to a plant, a growing media, soil, or planting area, by high or low pressure spraying. Suitable application means may also include atomizing, foaming, fogging, coating, and encrusting.

When treating plant seeds, the composition can be applied by low or high pressure spraying, coating, or immersion or other procedures as described herein.

In one embodiment, application of the composition is to soil or a growing medium for plants vulnerable to nematode disease as described herein.

In another embodiment, application of the composition is to plant seed as described herein.

The examples below are intended to exemplify the practice of embodiments of the disclosure but are by no means intended to limit the scope thereof.

### EXAMPLES

### Example 1—One-Pot Synthesis of 5-Alkylthio-1,2,4-thiadiazoles from Amidines and Carbon Disulfide

Results and Discussion

Our first attempt at synthesizing an ATTD used ethyl imidazole-1-carbodithioate (2) as the carbodithioate donor. This and other imidazole-1-carbodithioates have previously been used for the synthesis of carbamates and other thiocarbonyl compounds from heteroatom nucleophiles (Kumar et al., “Azole-carbodithioate Hybrids as Vaginal Anti-Candida Contraceptive Agents: Design, Synthesis and Docking Studies,” Eur. J. Med. Chem. 70:68-77 (2013); Sun et al., “1-(Methyldithiocarbonyl)imidazole: A reagent of S-methyldithiocarbonylation,” Synlett 1997:1279-1280 (1997); Mohanta et al., “1-(Methyldithiocarbonyl)imidazole: A useful Thiocarbonyl Transfer Reagent for Synthesis of Substituted Thioureas,” Tetrahedron 56:629-637 (2000), which are hereby incorporated by reference in their entirety). Using benzamidine hydrochloride (1a) with anhydrous potassium carbonate present as a base, S-ethyl N-(iminobenzyl)dithiocarbamate (3a) was isolated in 69% yield, which could then be reacted with N-chlorosuccinimide (NCS) to form 5-ethyl-3-phenyl-1,2,4-thiadiazole (4a) in 91% yield (63% yield from 1a) (FIG. 3).

Although imidazole-1-carbodithioates are useful carbodithioate donors, they decompose in the presence of moisture and air. Additionally, the synthesis of a new imidazole-1-carbodithioate would be required for each change to the S-alkyl group. Therefore, a method was sought that would allow the synthesis of ATTDs from amidines and alkyl halides using a simple combinatorial approach, without an imidazole-1-carbodithioate intermediate. Because amidines are highly nucleophilic (Taylor et al., “Amidines, Isothioureas, and Guanidines as Nucleophilic Catalysts,” Chem. Soc. Rev. 41:2109-2121 (2012), which is hereby incorporated by reference in its entirety), it was supposed that benzamidine would react with carbon disulfide in the presence of ethyl bromide to produce 3a, and the reaction proceeded in 27% isolated yield in THF. The possibility of using NCS in the same pot to oxidize 3a to 4a without the need to isolate the (iminobenzyl)dithiocarbamate was then explored. The reaction proceeded in 31% yield from the amidine.

The reaction was then optimized by exploring different solvents, bases, and oxidizers, as shown in Table 1. Since the goal was to perform the oxidation step in the same pot as the formation of 3a, solvents that could tolerate many oxidants were chosen (Table 1, Entries 1-6). Of the solvents selected, acetonitrile (ACN) gave the highest yield using K₂CO₃ as the base. When the reaction was performed in methanol (Table 1, Entry 6), the sole isolated product was 5-methoxy-3-phenyl-1,2,4-thiadiazole.

TABLE 1
Optimization of the One-pot, Two-step Synthesis
of 5-ethylthio-3-phenyl-1,2,4-thiadiazole

| | | | | Eq. | | | |
|---|---|---|---|---|---|---|---|
| Entry | Solvent | Timeª | | CS₂/EtBr^{b} | Base (Eq.)^{c} | Ox. (Eq.)^{d} | Yield^{e} |
| 1 | THF | 6 | h | 1.05/1.05 | K₂CO₃ (2.5) | NCS (1.05) | 31 |
| 2 | ACN | 6 | h | 1.05/1.05 | K₂CO₃ (2.5) | NCS (1.05) | (39) |
| 3 | Diglyme | 6 | h | 1.05/1.05 | K₂CO₃ (2.5) | NCS (1.05) | (12) |
| 4 | DMF | 6 | h | 1.05/1.05 | K₂CO₃ (2.5) | NCS (1.05) | nd |
| 5 | CHCl₃ | 6 | h | 1.05/1.05 | K₂CO₃ (2.5) | NCS (1.05) | (6) |
| 6 | MeOH | 6 | h | 1.05/1.05 | K₂CO₃ (2.5) | NCS (1.05) | 0^{f} |
| 7 | THF | 6 | h | 1.05/1.05 | DBU (2.5) | NCS (1.05) | 38 |
| 8 | Diglyme | 6 | h | 1.05/1.05 | DBU (2.5) | NCS (1.05) | 25 |
| 9 | CHCl₃ | 6 | h | 1.05/1.05 | DBU (2.5) | NCS (1.05) | 30 |
| 10 | ACN | 6 | h | 1.05/1.05 | DBU (2.5) | NCS (1.05) | 51 |
| 11 | ACN | 6 | h | 1.05/1.05 | K₃PO₄ (2.5) | NCS (1.2) | (21) |
| 12 | ACN | 6 | h | 1.05/1.05 | Et₃N (2.1) | NCS (1.2) | 0 |
| 13 | ACN | 6 | h | 1.05/1.05 | DABCO (2.1) | NCS (1.2) | 0 |
| 14 | ACN | 6 | h | 1.05/1.05 | DBN (2.1) | NCS (1.2) | (47) |
| 15 | ACN | 6 | h | 1.2/1.4 | DBU (2.5) | NCS (1.05) | 60 |
| 16 | ACN | 6 | h | 1.2/1.4 | DBU (2.1) | NCS (1.05) | 62 |
| 17 | ACN | 6 | h | 1.2/1.4 | DBU (2.1) | NCS (1.2) | 64 |
| 18 | ACN | 6 | h | 1.2/1.4 | DBU (1.1) | NCS (1.2) | (25) |
| 19 | ACN | 6 | h^{g} | 1.2/1.4 | DBU (2.1) | NCS (1.2) | 43 |
| 20 | ACN | 4 | h | 1.2/1.4 | DBU (2.1) | NCS (1.2) | 55 |
| 21 | ACN | 16 | h | 1.2/1.4 | DBU (2.1) | NCS (1.2) | 67 |
| 22 | ACN | 16 | h | 2/3 | DBU (2.1) | NCS (1.2) | 47 |
| 23 | ACN | 16 | h | 1.2/1.4 | DBU (2.1) | NBS (1.2) | 55 |
| 24 | ACN | 16 | h | 1.2/1.4 | DBU (2.1) | PIDA (1.2) | 62 |
| 25 | ACN | 16 | h | 1.2/1.4 | DBU (2.1) | PIFA (1.2) | 65 |
| 26 | ACN | 16 | h | 1.2/1.4 | DBU (2.1) | I₂ (1.2) | 59 |
| 27 | ACN | 16 | h | 1.2/1.4 | DBU (2.1) | Chloranil (1.2) | (36) |
| 28 | ACN | 16 | h | 1.2/1.4 | DBU (2.1) | H₂O₂ (1.2) | (23) |
| 29 | ACN | 16 | h | 1.2/1.4 | DBU (2.1) | TCICA (0.4) | (43) |
| 30 | ACN | 16 | h | 1.2/1.4 | DBU (2.1) | DBDMH (0.6) | (56) |
| 31 | ACN | 16 | h | 1.2/1.4 | DBU (2.1) | Cu²⁺ (0.05) + air^{g} | (6) |

Optimization reactions were performed using 0.1 mmol of benzamidine hydrochloride in 1 mL of solvent.
^{a}Reaction time prior to addition of oxidant.
^{b}Equivalents of carbon disulfide and ethyl bromide, respectively.
^{c}Base (equivalents). DABCO: 1,4-diazabicyclo[2.2.2]octane; DBN: 1,5-diazabicyclo(4.3.0)non-5-ene.
^{d}Oxidant (equivalents). NBS: N-bromosuccinimide; PIDA: phenyl-iodine(III) diacetate; TCICA: trichloroisocyanuric acid; DBDMH: 1,3-dibromo-5,5-dimethyl-hydantoin.
^{e}Yields in parentheses are estimated by GCMS using 4-chlorobenzonitrile as an internal reference, otherwise the isolated yield is given.
^{f}The major product was 5-methoxy-3-phenyl-1,2,4-thiadiazole, isolated in 38% yield.
^{g}Tetrakis(acetonitrile)copper(I) hexafluorophosphate was used as the copper(II) source.

Because it was speculated that the low solubility of K₂CO₃ in many solvents might limit the formation of the free amidine, DBU was explored as a relatively strong neutral organic base (Table 1, Entries 7-10), producing higher yields of 4a than potassium carbonate in solvents used, though ACN still produced the best results. Several bases were also explored, including anhydrous K₃PO₄, triethylamine, DABCO, and DBN (Table 1, Entries 11-14). DBU remained the best base of those tested. Neither triethylamine nor DABCO produced any 3a as determined by TLC, likely because benzamidine is a stronger base than either of these amines, so the free amidine was never formed. In many cases, the main product detected by GCMS was 4-chlorobenzonitrile, likely produced by the direct oxidation of the amidine to the nitrile. It should be noted that DBU may also assist in the formation of 3a as a catalyst, given that cyclic amidines are known to readily add to carbon disulfide (Ang et al., “Contrasting Reactivity of CS2 with Cyclic vs. Acyclic Amidines,” Eur. J. Org. Chem. 2015:7334-7343 (2015), which is hereby incorporated by reference in its entirety).

Table 1, Entries 15-22 explore the effects of changing the reaction time or temperature, as well as the stoichiometric ratios of the base, oxidant, and other reactants. Table 1, Entry 21 shows the best results obtained. The increased amount of carbon disulfide and ethyl bromide was beneficial as diethyl trithiocarbonate was isolated in small amounts as a side product. The base-mediated formation of trithiocarbonates from carbon disulfide and alkyl halides is known in literature (Fallah-Mehrjardi, M., “Review of the Organic Trithiocarbonates Synthesis,” Monatsh. Chem. 149:1931-1944 (2018), which is hereby incorporated by reference in its entirety). However, increasing the ratio of carbon disulfide and ethyl bromide further resulted in a decreased yield (Table 1, Entry 22), likely due to the formation of DBU·CS₂ adducts (Ang et al., “Contrasting Reactivity of CS2 with Cyclic vs. Acyclic Amidines,” Eur. J. Org. Chem. 2015:7334-7343 (2015), which is hereby incorporated by reference in its entirety). When other common oxidants were used (Table 1, Entries 23-30), lower yields of 4a were obtained compared to NCS. PIDA, PIFA, and iodine all provided qualitatively cleaner reactions by TLC which may be of importance in some applications. Indeed, hypervalent iodine reagents have previously been shown to be excellent oxidants for the formation of 5-amino-1,2,4-thiadiazoles (Mariappan et al., “Hypervalent Iodine(III) Mediated Synthesis of 3-substituted 5-amino-1,2,4-thiadiazoles Through Intramolecular Oxidative S—N Bond Formation,” J. Org. Chem. 81:6573-6579 (2016), which is hereby incorporated by reference in its entirety). However, the greater cost of these oxidants, combined with their lower yields, did not justify their exploration for the scope of the reaction. An oxidation using catalytic copper(II) and air as an oxidant was also attempted, as similar systems have been used for the synthesis of 1,2,4-oxadiazoles (Kuram et al., “Copper-catalyzed Direct Synthesis of 1,2,4-oxadiazoles From Amides and Organic Nitriles by Oxidative N—O Bond Formation,” Eur. J. Org. Chem. 2016:438-442 (2016), which is hereby incorporated by reference in its entirety). However, these oxidations often require heating, and under the conditions of the reaction, the oxidation was inefficient.

After optimizing the conditions, the scope of the reaction was explored using a variety of amidines and alkyl halides (FIG. 4). The reaction worked well with a wide variety of alkyl bromides and iodides. Using ethyl iodide in place of ethyl bromide to form 4a did not result in a significant improvement of the reaction yield. Isopropyl iodide provided moderate yields of 4d, particularly when compared to 4e, which used isobutyl bromide. Both allyl and benzyl bromides resulted in good yields (4g and 4e). ATTDs 4m, 4n, 4s, and 4t were synthesized with heteroatoms present in the primary halide used in the reaction, and the expected products were isolated in good yield.

A variety of amidines were also explored, including benzamidines substituted with either electron-donating (4q, 4r, and 4u) or electron-withdrawing (4j, 4k, 4o, 4p, and 4v) groups. The yield of the reaction did not appear to depend heavily on the functional groups present on the aromatic ring. While anhydrous amidines were typically used, 4p was synthesized from the amidine hydrochloride dihydrate without any issues. Several heteroarylamidines also provided good yields (4w, 4x, and 4y), though the yields (51-62%) were lower than most of those obtained for substituted benzamidines.

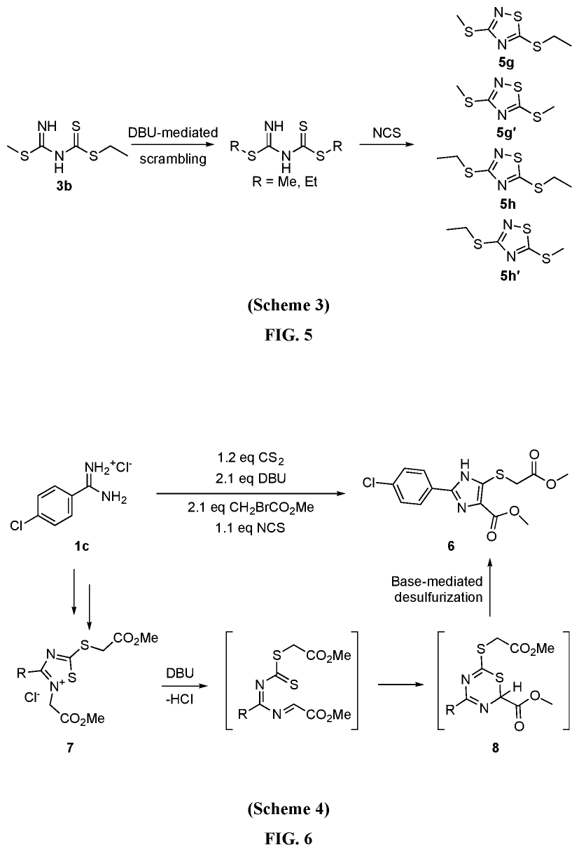

Given the success with aryl amidines, the aromatic ring was then replaced with an alkyl group (5a, 5b, and 5c), which did not significantly reduce the yield of the reaction. When ethyl 2-amidinoacetate or malonamamidine were used as the starting amidines, the resultant products (5d and 5e, respectively) were obtained in good yields. Based on NMR data, these products assume the 3-methylidene-1,2,4-thiadiazoline tautomeric form in both chloroform and DMSO, as opposed to the more frequently observed thiadiazole. The use of the related compounds O-methylisouronium hemisulfate, S-methylisothiouronium hemisulfate, and N,N-dimethylguanadinium hemisulfate as starting materials was then explored. Products 5f and 5i were obtained in modest yields. Given the low observed solubility of these hemisulfate salts in acetonitrile, modifying the reaction solvent to 10% N,N-dimethylpropyleneurea (DMPU) in ACN improved yields slightly. Unfortunately, when S-methylisothiouronium hemisulfate was used, a roughly stoichiometric mix of 5-ethylthio-3-methylthio-1,2,4-thiadiazole (5g), 3,5-bis(methylthio)-1,2,4-thiadiazole (5g′), 3,5-bis(ethylthio)-1,2,4-thiadiazole (5h), and 3-ethylthio-5-methylthio-1,2,4-thiadiazole (5h′) was obtained, suggesting that in the corresponding intermediate S,S′-dialkly (dithiocarboxy)isothiourea 3b, the alkylthio groups were easily scrambled (FIG. 5). These closely related thiadiazoles could not be separated by column chromatography. Using S-ethylisothiouronium bromide, 5h could be obtained in moderate yield as scrambling no longer led to different products. Other substituted guanidines were used, namely, an N-benzylpiperazine derivative, and N^{α}-benzoyl-L-arginine ethyl ester hydrochloride which produced the expected products (5j and 5k) in acceptable yields.

Unfortunately, the simplest amidine salt used, formamidine acetate, did not produce any of the expected product 5l. This is not particularly surprising given that 1,2,4-thiadiazoles lacking 3- and 5-substitutents are very sensitive to acids and bases and oxidizing and reducing agents (Kurzer, F., “1,2,4-Thiadiazoles,” Adv. Heterocycl. Chem. 5:119-204 (1965), which is hereby incorporated by reference in its entirety). Likewise, the use of trifluoroacetamidine did not provide the desired product 5m, perhaps because of the lowered nucleophilicity of the parent amidine resulting from the strongly electron-withdrawing trifluoromethyl group.

Interestingly, the use of methyl bromoacetate as the alkyl halide did not give the expected ATTD, but instead gave as 45% yield (calculated from methyl bromoacetate as the limiting reagent) of the substituted imidazole 6 when starting from 4-chlorobenzamidine, as shown in FIG. 6. Previous work suggests that the formation of the imidazole occurs through a dialkylated N-(α-aminobenzylidene) dithiocarbamate, which is oxidized to the thiadiazolium 7, followed by base-mediated ring-opening and a [4+2] electrocyclization to produce the 2H-1,3,5-thiadiazine 8, and finally base-mediated desulfurization (Rolfs & Liebscher, “Versatile Novel Syntheses of Imidazole,” J. Org. Chem. 62:3480-3487 (1997), which is hereby incorporated by reference in its entirety).

TABLE 2
Formation of 5-methoxy-1,2,4-thiadiazoles 10 and 2-methoxy-
1,3,5-triazines 11 from Amidines in Methanol

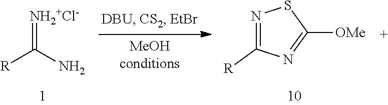

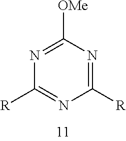

| R | Conditions^{a} | Product (Yield) |
|---|---|---|
| phenyl | A | 10a (26%) + 11a (9%) |
| phenyl | B | 11a (33%) |
| 4-fluorophenyl | A | 10b (30%) + 11b (12%) |
| 4-fluorophenyl | B | 11b (45%) |
| 4-chlorophenyl | A | 10c (42%) + 11c (8%) |
| 4-chlorophenyl | B | 11c (39%) |
| 3-methoxyphenyl | A | 10d (49%) + 11d (6%) |
| 3-methoxyphenyl | B | 11d (34%) |
| 4-nitrophenyl | A | 10e (64%) |

^{a}Condition A: DBU (2.05 eq), CS₂ (1.5 eq), and 1 (1 eq) in MeOH, 22 °C.,
120 min; then EtBr (1.5 eq), 22 °C., 6 hr, then NCS (1.1 eq), 0° C., 30 min.
Condition B: DBU (4.1 eq), CS2 (1 eq), and EtBr (273 mg, 2.5 mmol) in
MeOH, 22° C., hr; then reflux, 6 hr.

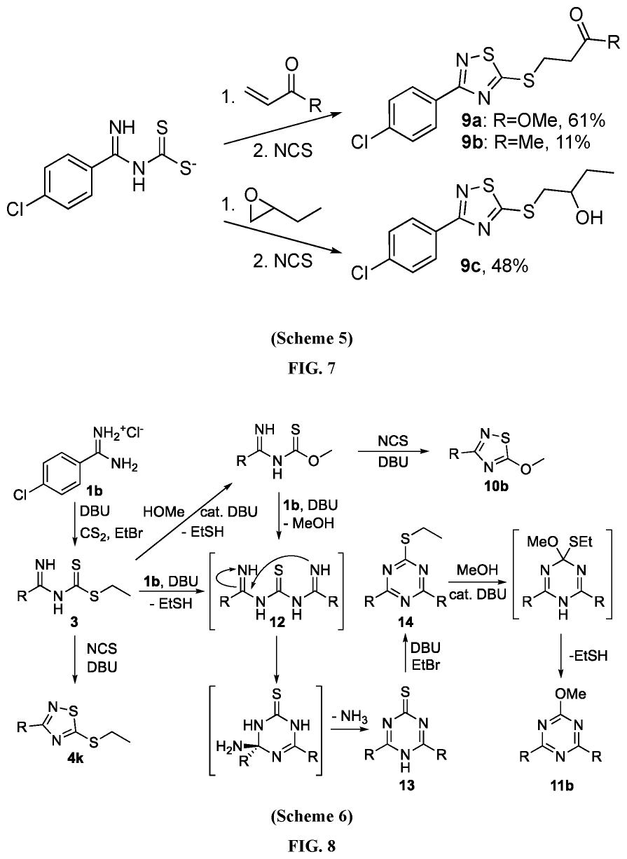

It was then explored whether the scope of the reaction could be broadened through the use of other electrophiles (FIG. 7). The conjugate addition of thiolates to Michael acceptors is well-established (Nair et al., “The Thiol-Michael Addition Click Reaction: A Powerful and Widely Used Tool in Materials Chemistry,” Chem. Mater. 26:724-744 (2014); Lowe, A. B., “Thiol-ene “Click” Reactions and Recent Applications in Polymer and Materials Synthesis: A First Update,” Polym. Chem. 5:4820-4870 (2014); Sinha & Equbal, “Thiol-ene Reaction: Synthetic Aspects and Mechanistic Studies of an Anti-Markovnikov-selective Hydrothiolation of Olefins,” Asian J. Org. Chem. 8:32-47 (2019), which are hereby incorporated by reference in their entirety), and therefore the use of methyl acrylate and methyl vinyl ketone in lieu of an alkyl halide was explored. The yields of the ester 9a and ketone 9b were lower than those obtained when using an alkyl halide; however, the reaction conditions were not developed with the thiol-ene click reaction in mind, and future optimization would likely improve these yields. 1,2-Epoxybutane also reacted readily with the 4-chlorobenzamidine-carbon disulfide adduct to form 2-hydroxybut-1-yl compound 9c in moderate yield.

Because 5-methoxy-3-phenyl-1,2,4-thiadiazole (10a) was produced during an optimization trial in methanol (Table 1, Entry 6), the feasibility of using a modification of the ATTD synthesis to produce other 5-alkoxy-1,2,4-thiadiazoles using the reaction conditions optimized for ATTDs was explored. By using DBU as the base instead of potassium carbonate, and changing the solvent to methanol the reaction of 4-chlorobenzamidine led to the production of 5-methoxy-3-(4-chlorophenyl)-1,2,4-thiadiazole (10b), as well as a small quantity of 4,6-bis(4-chlorophenyl)-2-methoxy-1,3,5-triazine (11b). Table 2 shows the yields of 10 and 11 from several different benzamidines. Because the formation of 11 does not require the use of an oxidant, the triazine could be obtained in modest yields from amidine 1, carbon disulfide, ethyl bromide, and DBU by simply heating the reaction to reflux to effect the elimination of ethanethiol and ammonia. Alternatively, using NCS was effective for forming 5-methoxy-1,2,4-thiadiazoles 10, and Scheme 6 shows the synthesis of 10 and 11 from 1. Unfortunately, the production of 10 was frequently accompanied by some of the triazine 11, which, in the examples provided, had similar retention factors to 10, complicating purification efforts. Additionally, the yields obtained for these 5-alkoxy-1,2,4-thiadiazoles were substantially lower than those for ATTDs, suggesting that optimization of this reaction is necessary before this method becomes attractive for the synthesis of these compounds.

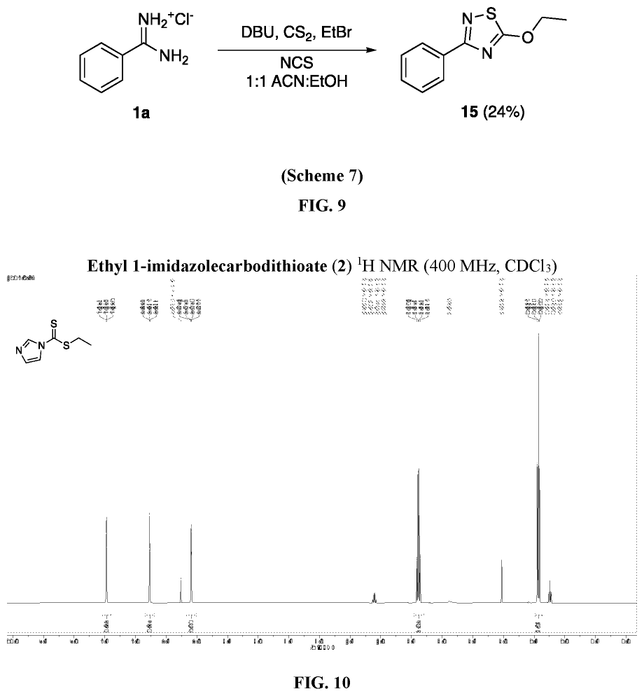

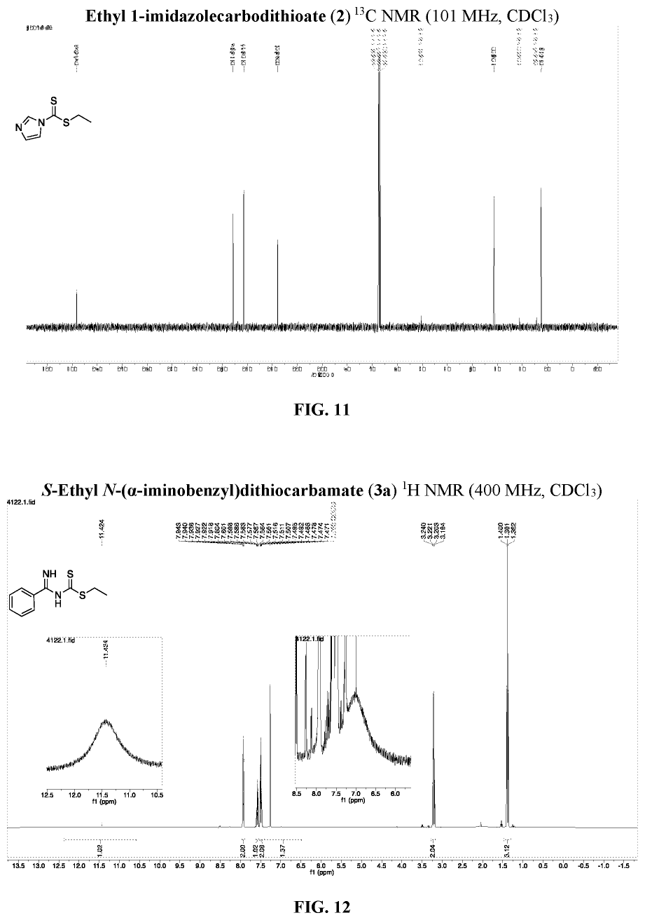

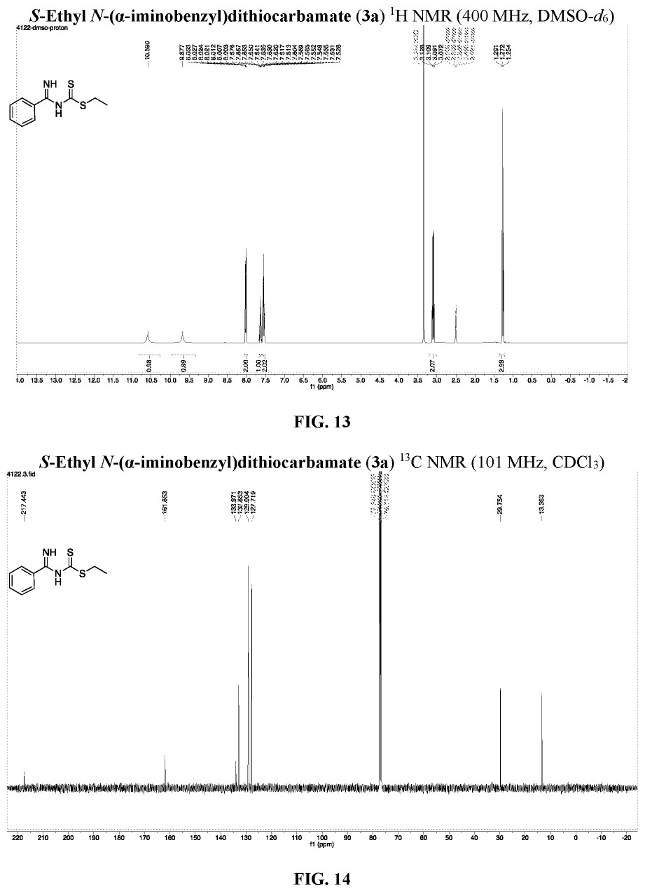

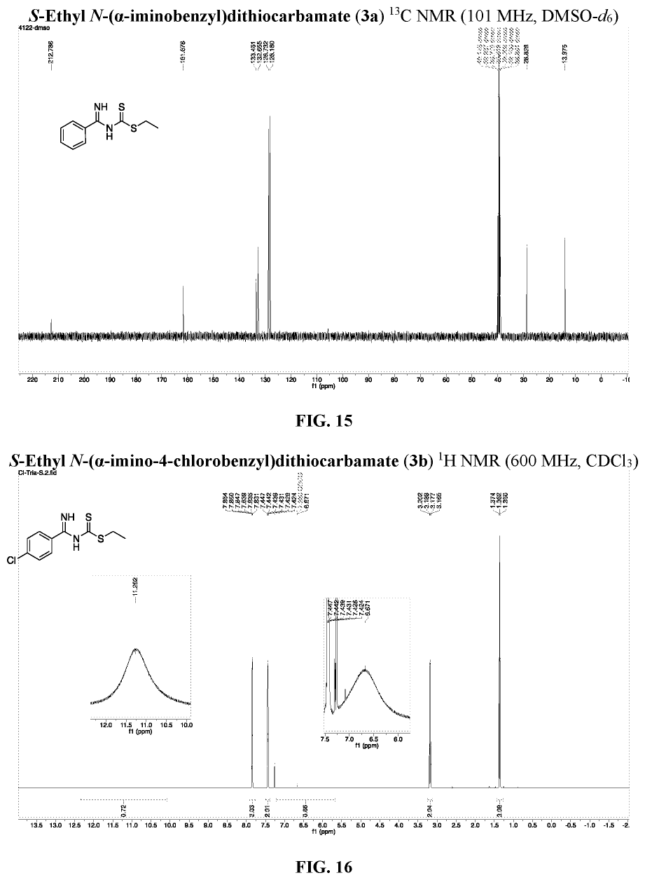

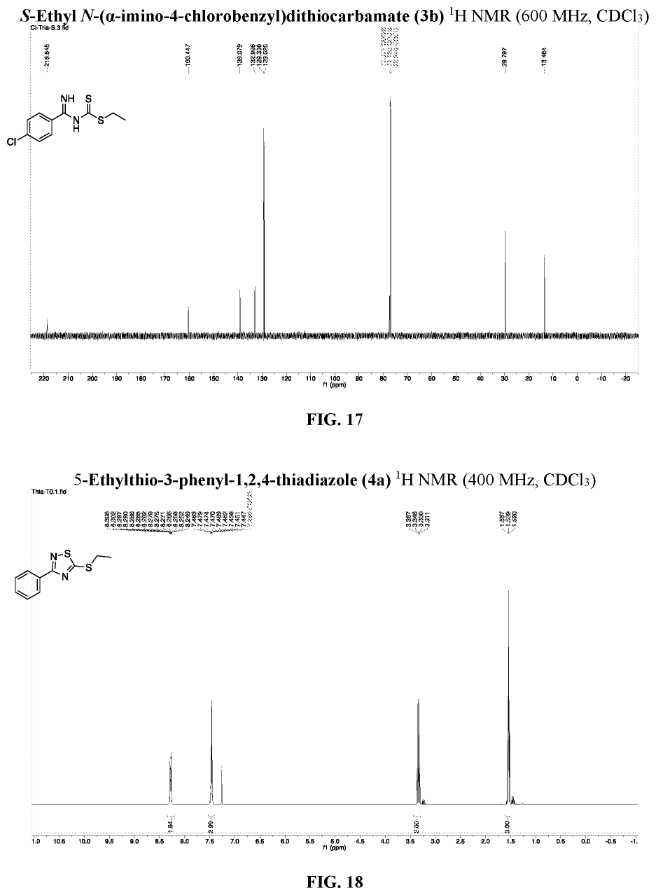

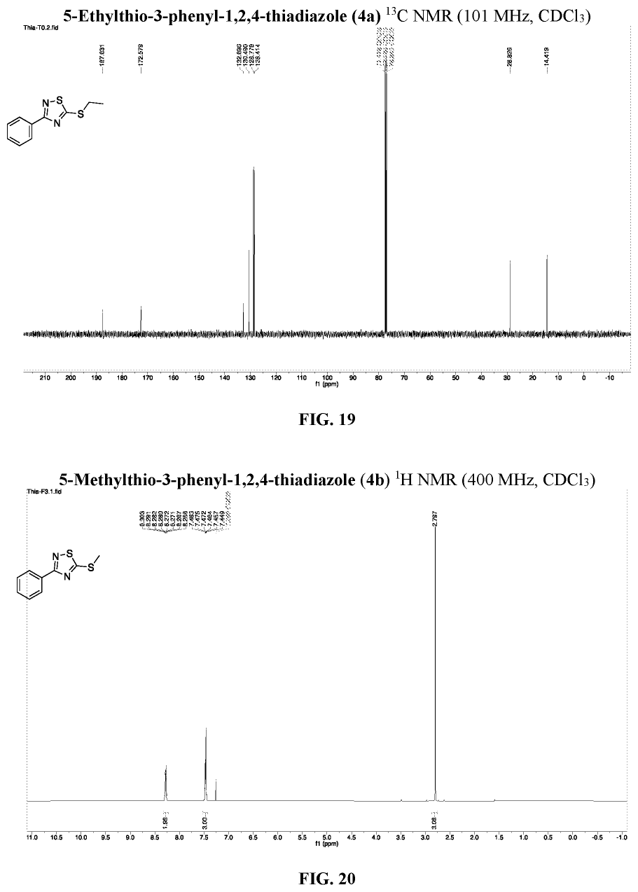

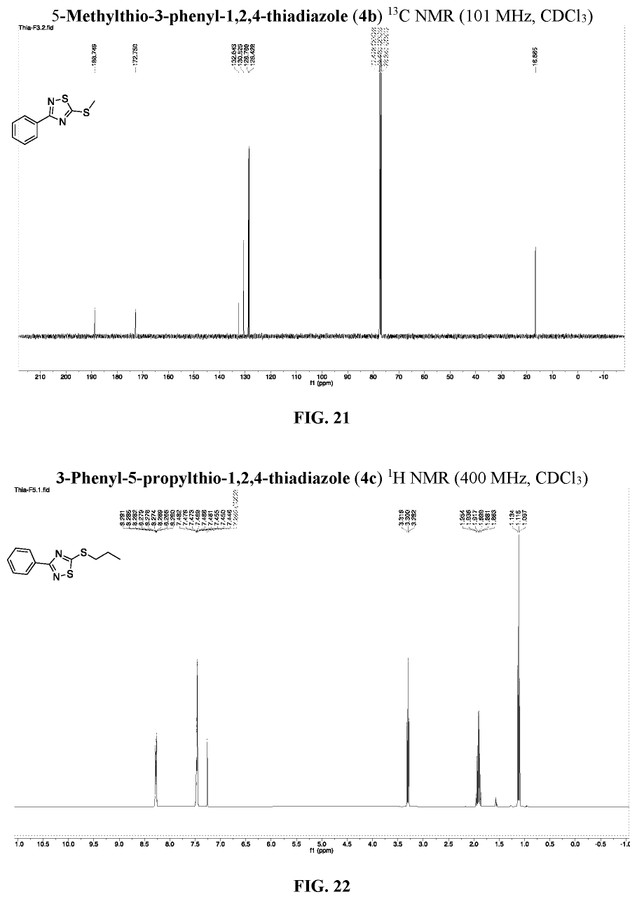

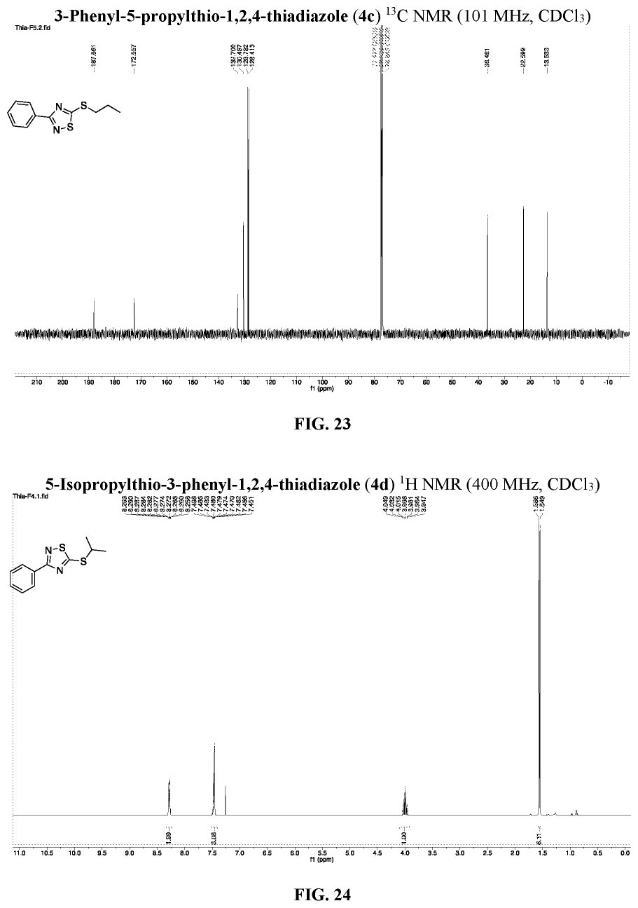

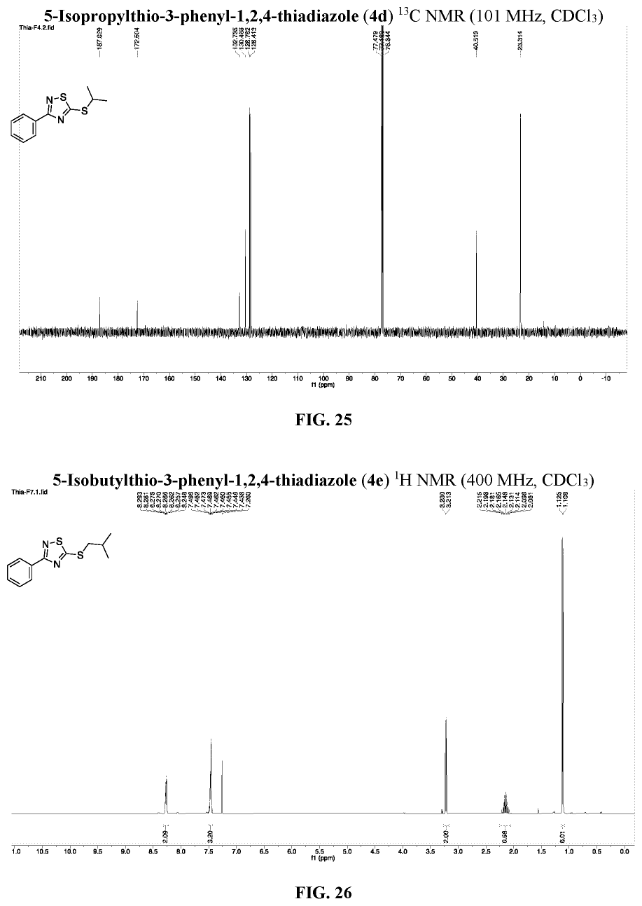

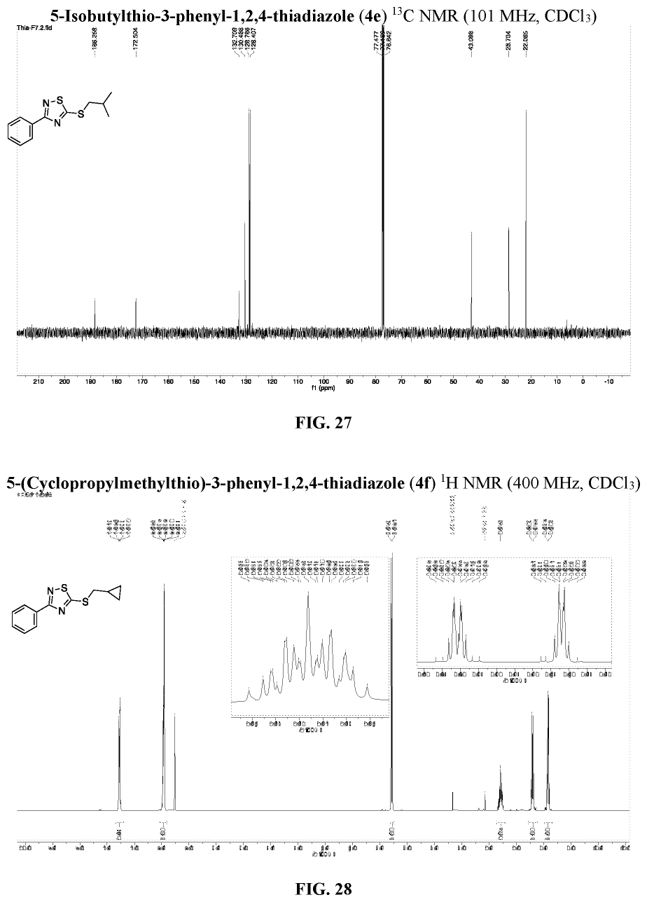

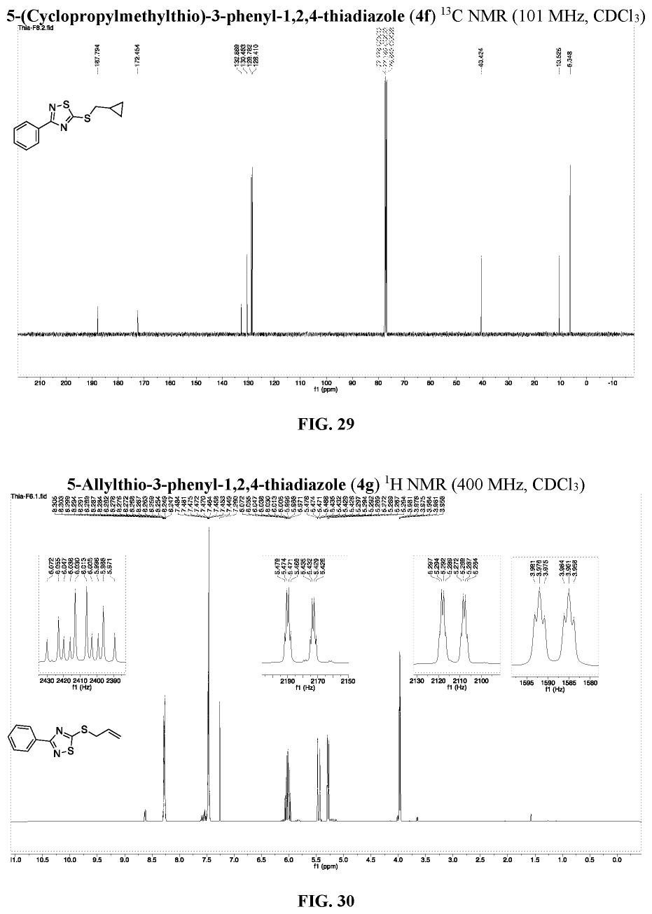

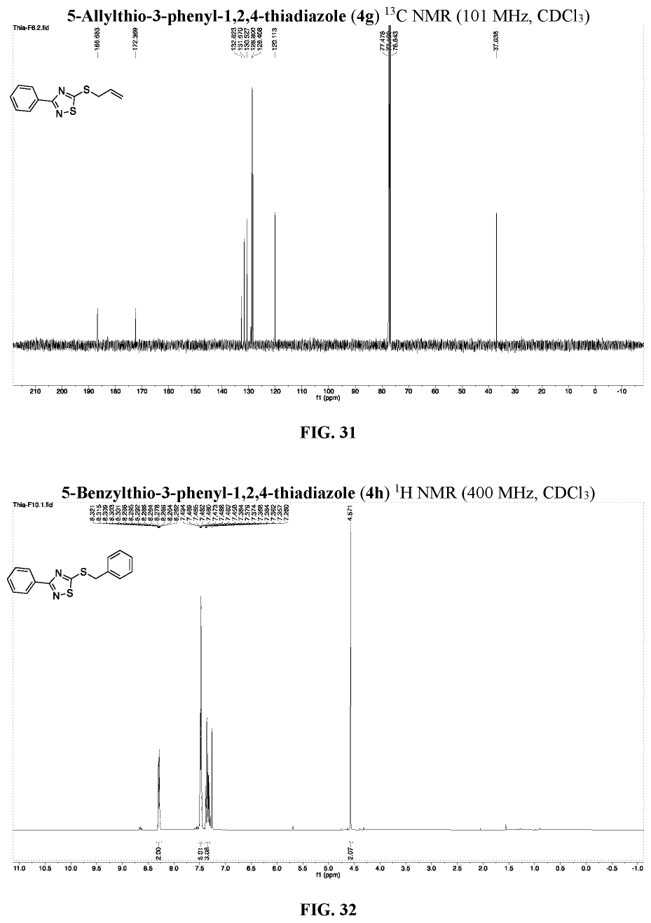

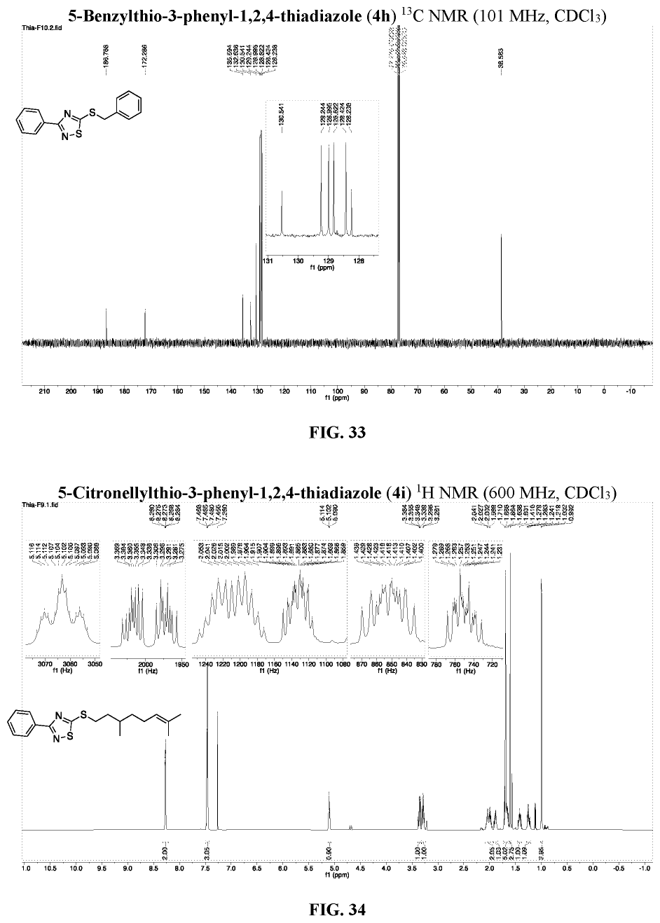

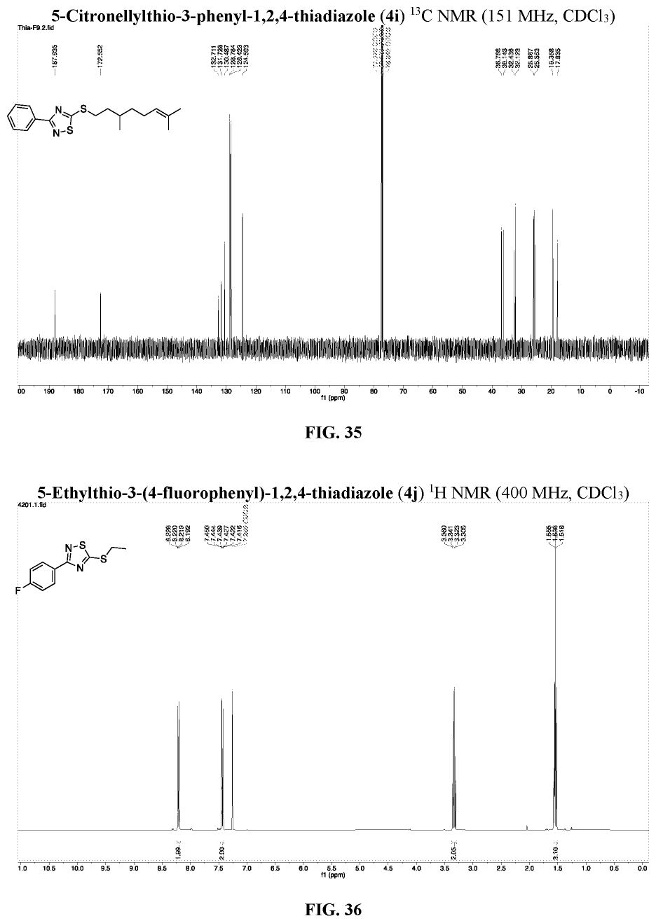

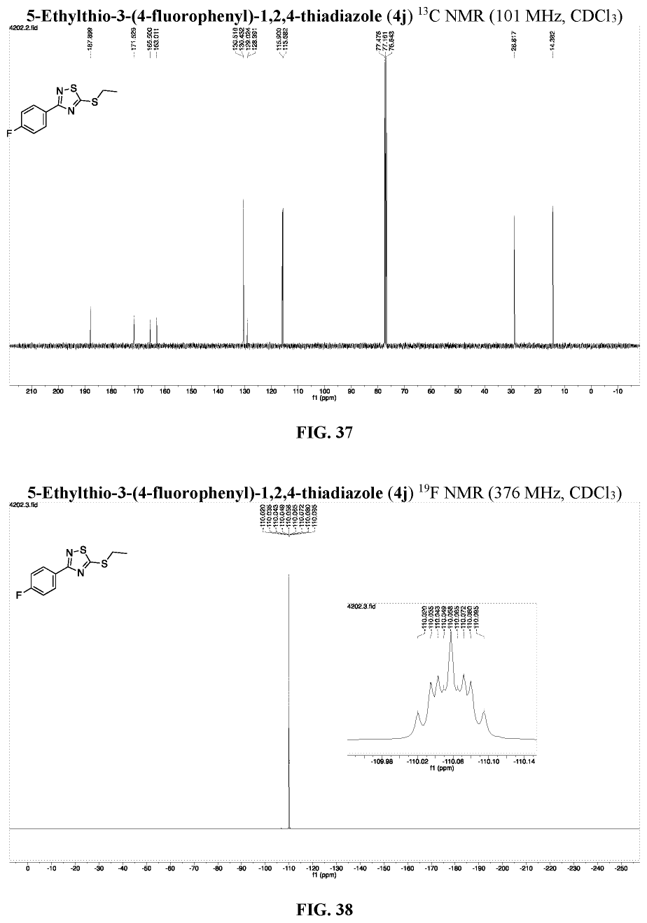

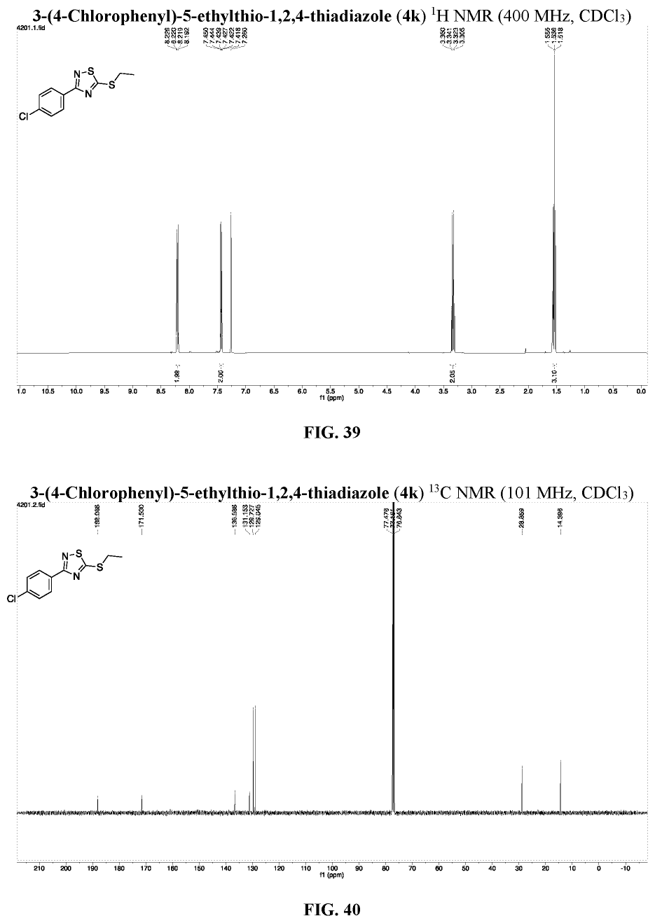

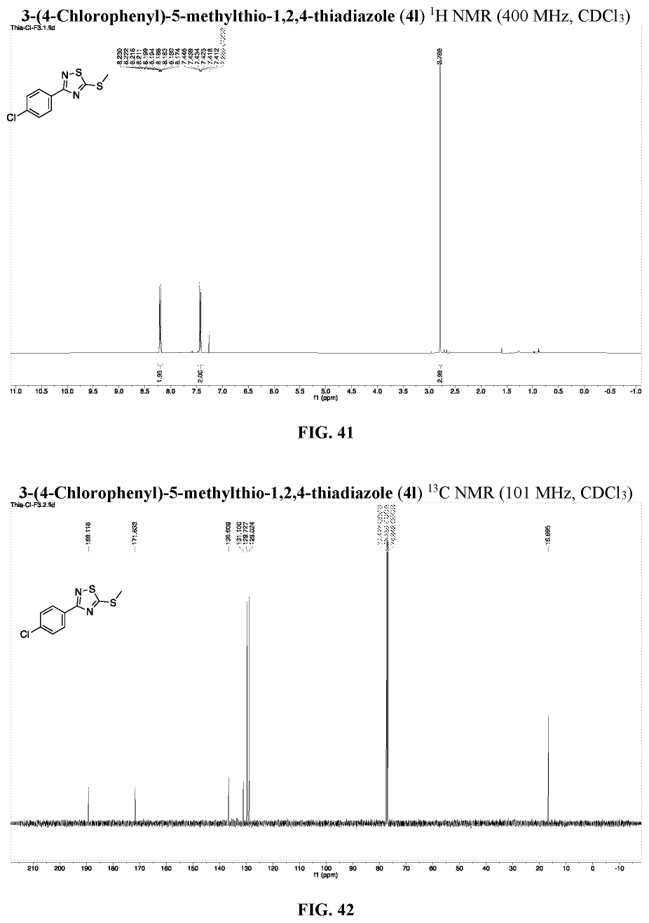

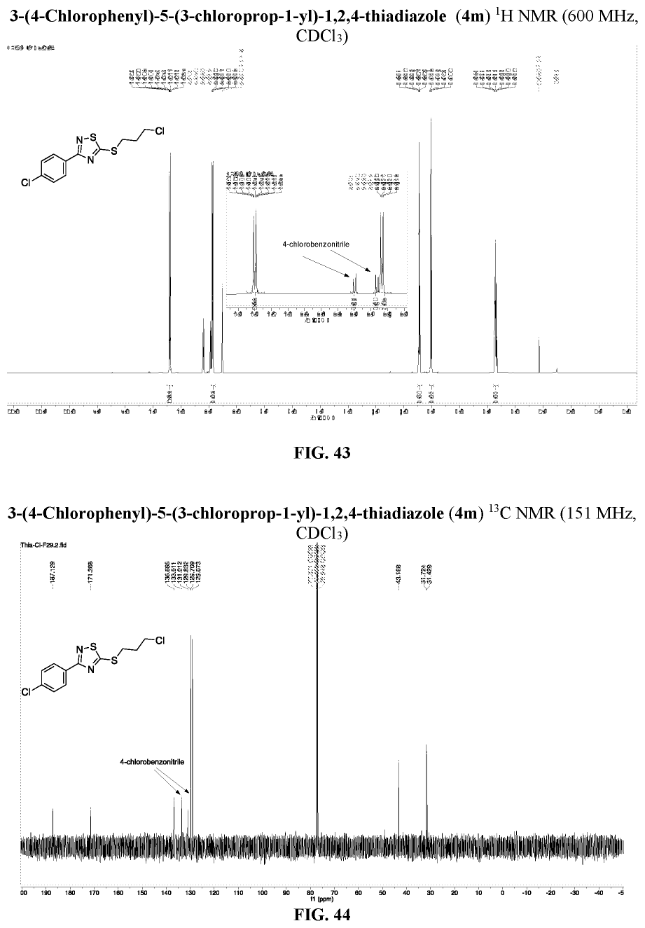

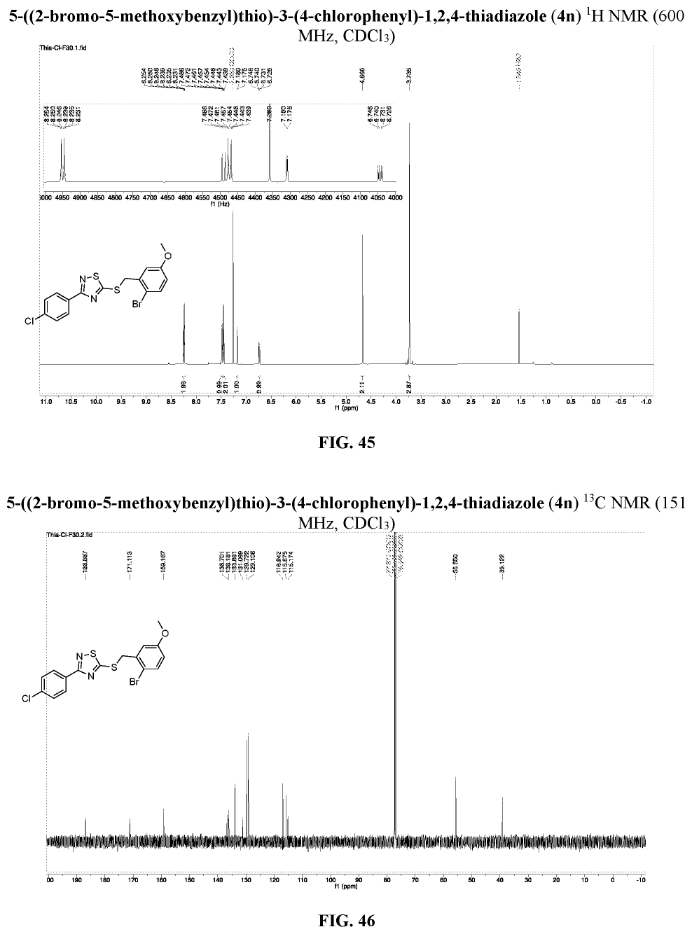

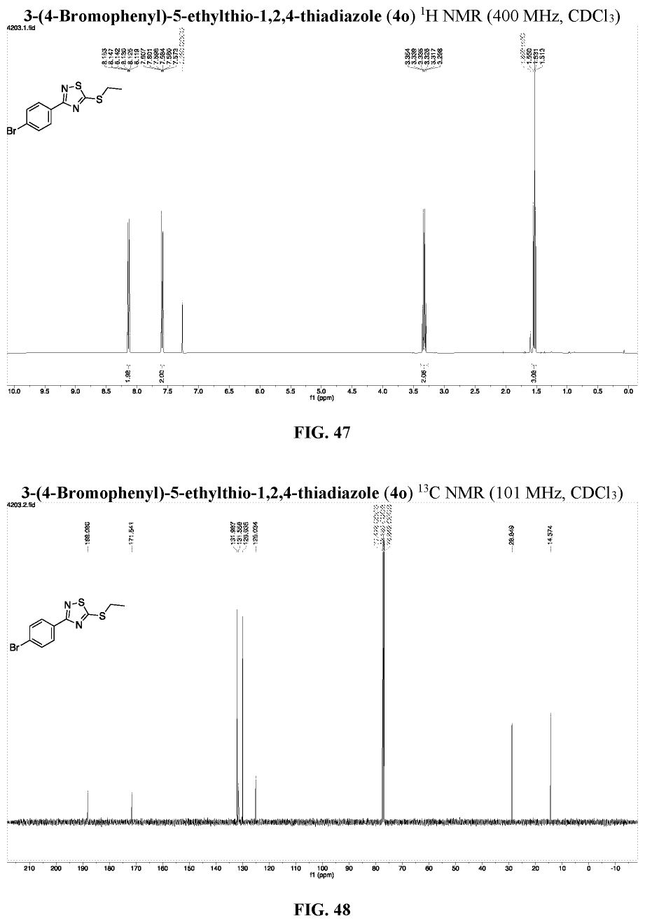

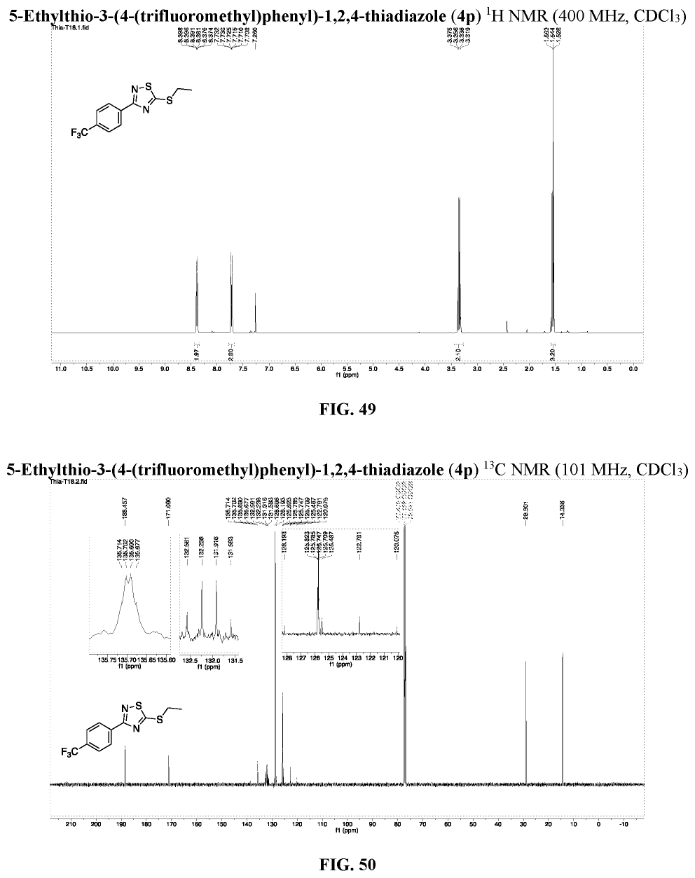

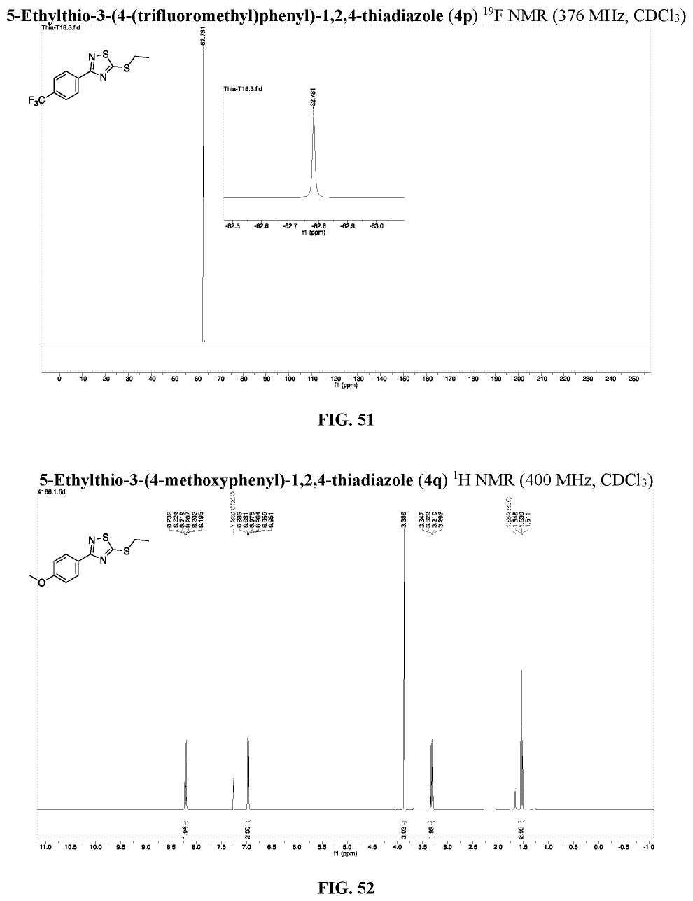

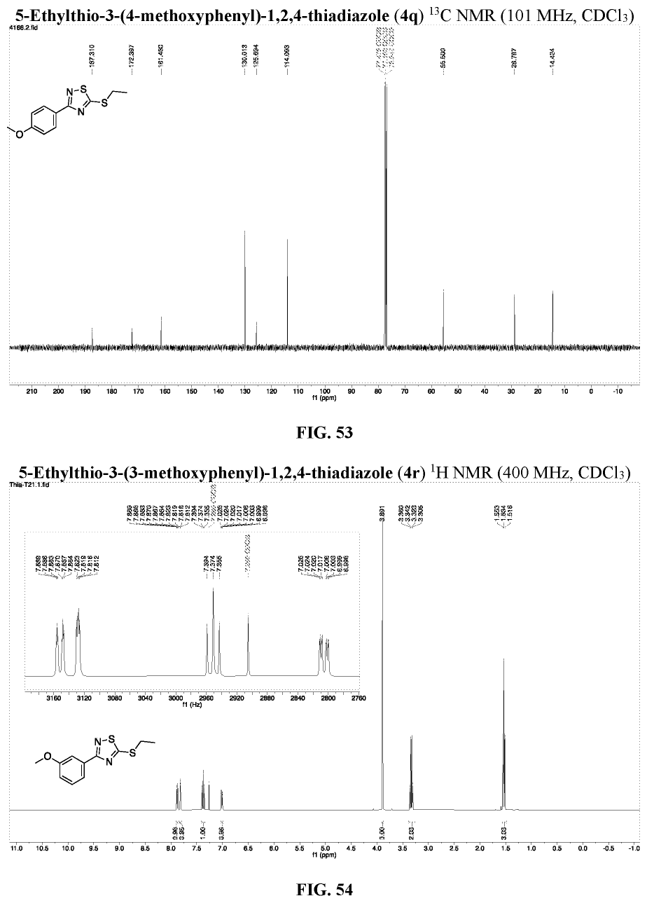

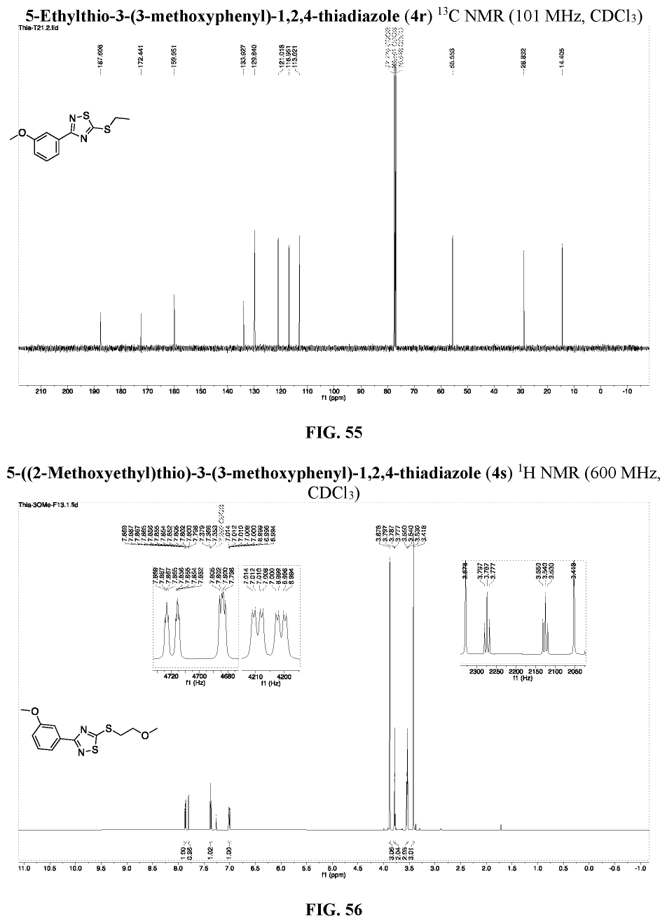

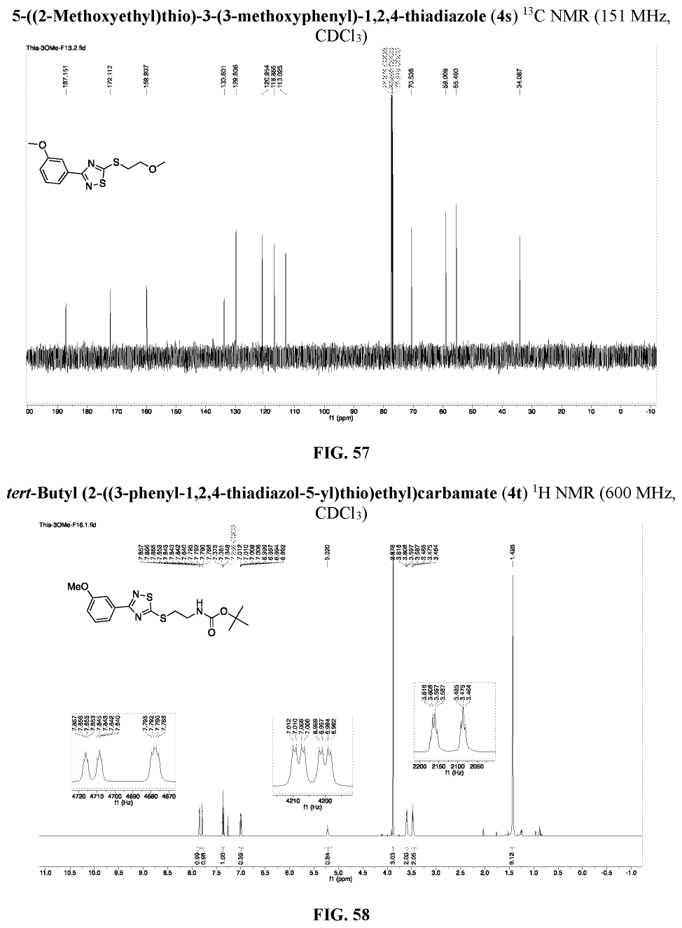

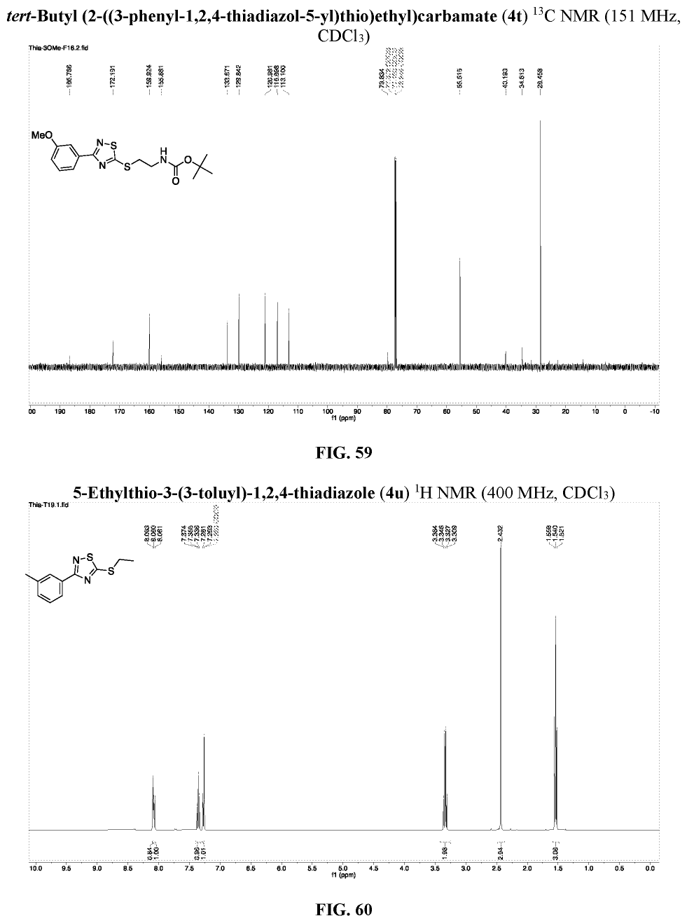

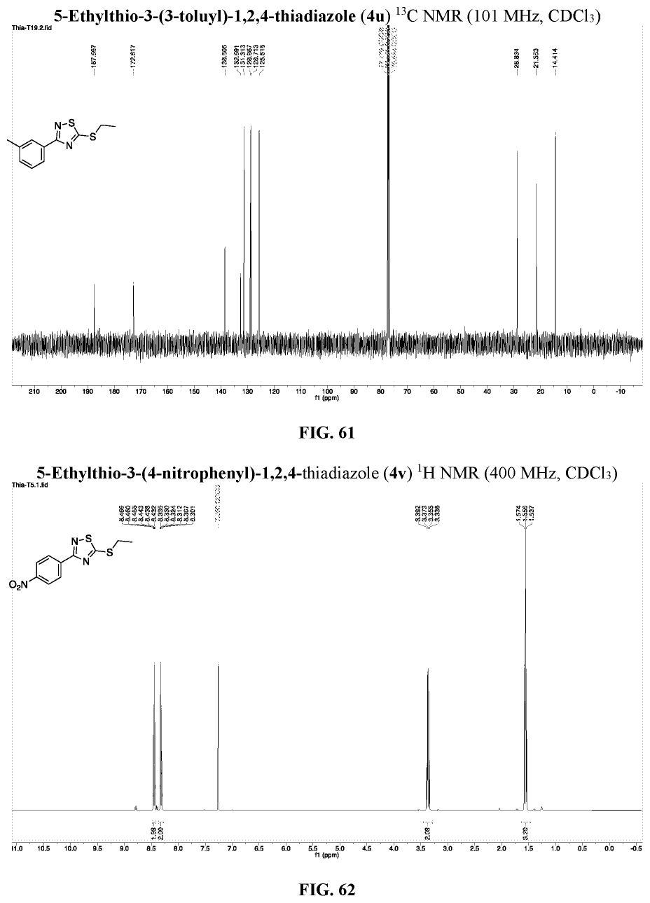

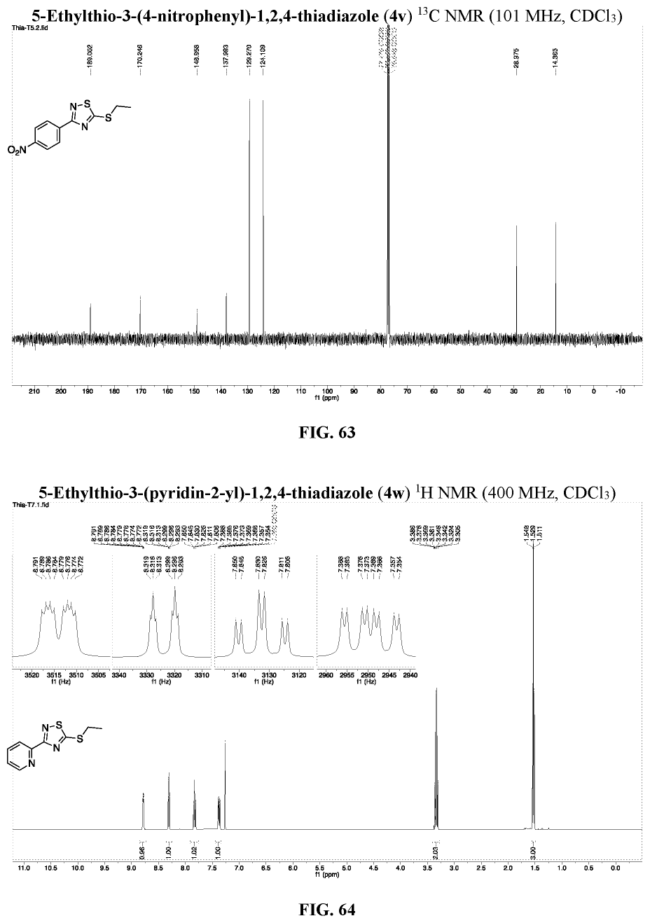

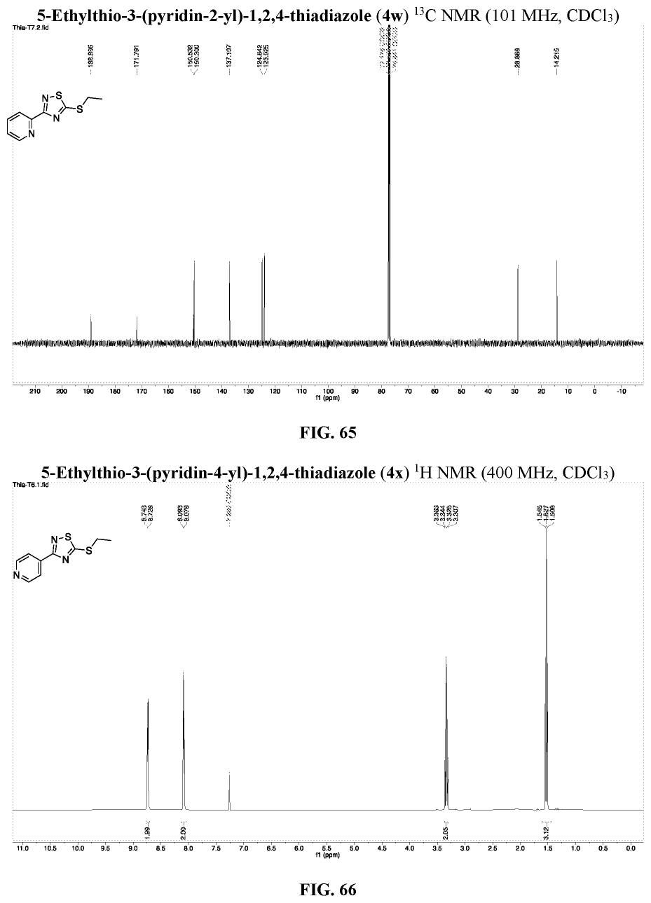

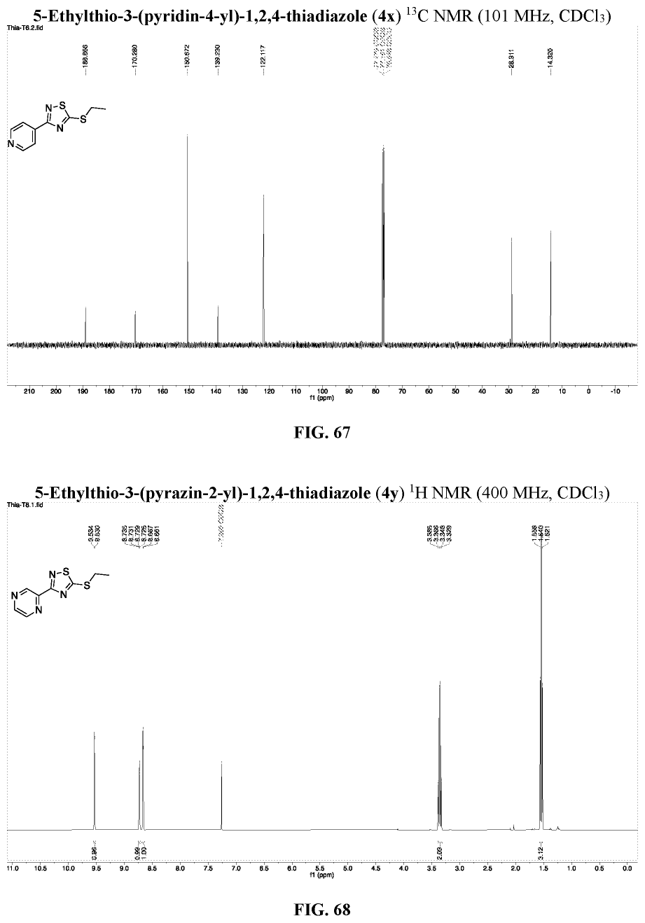

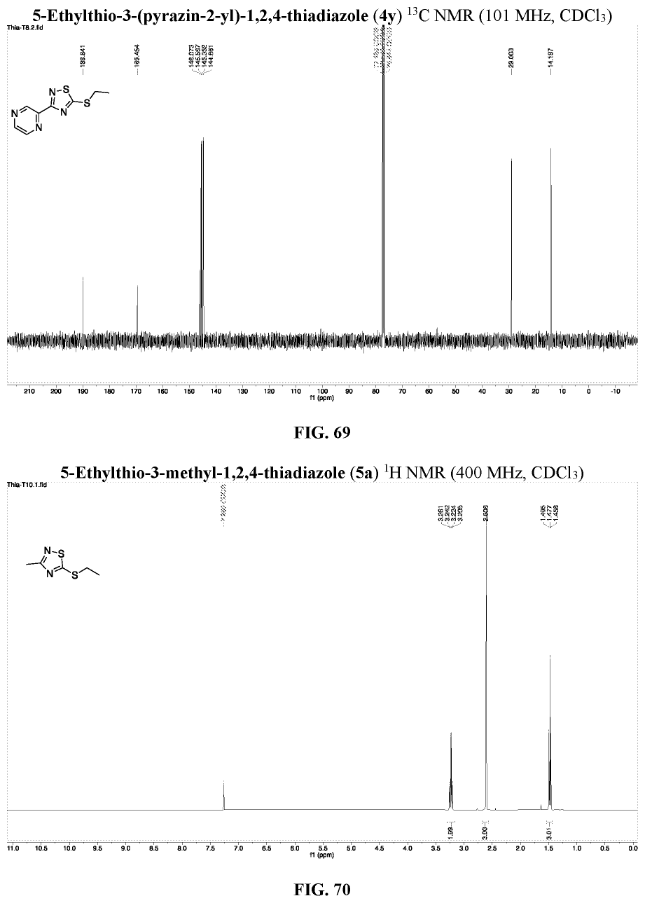

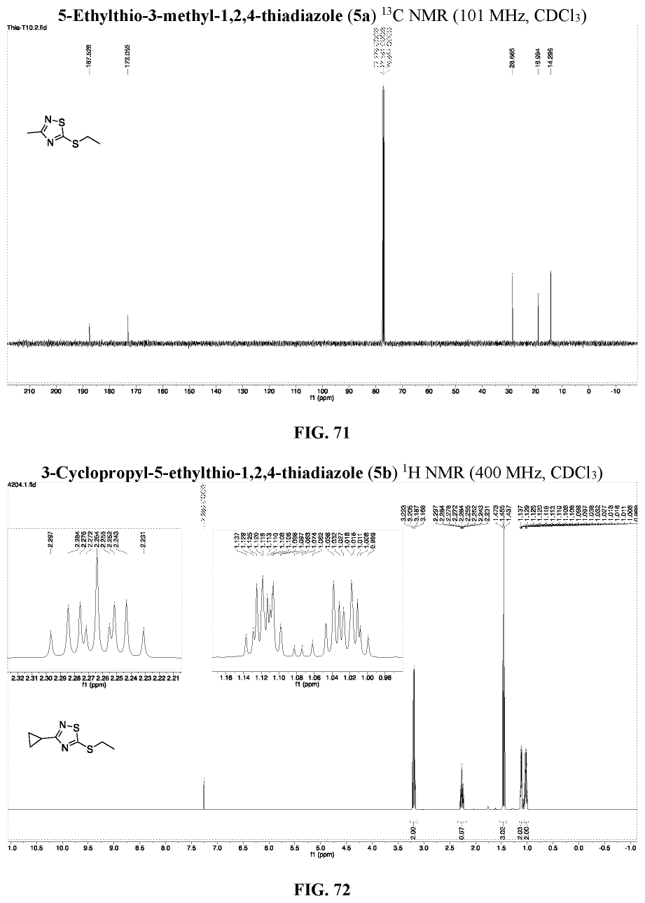

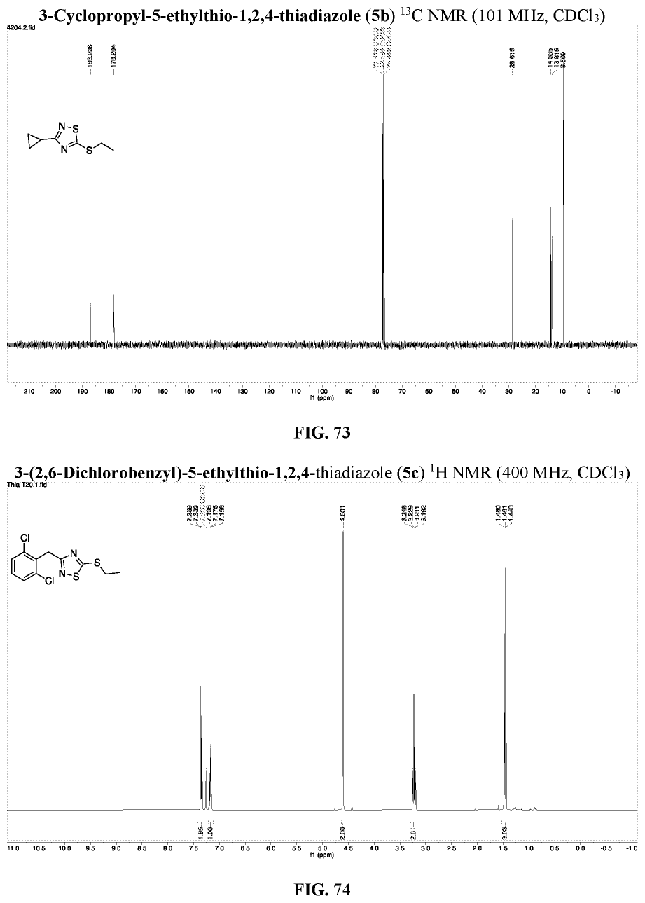

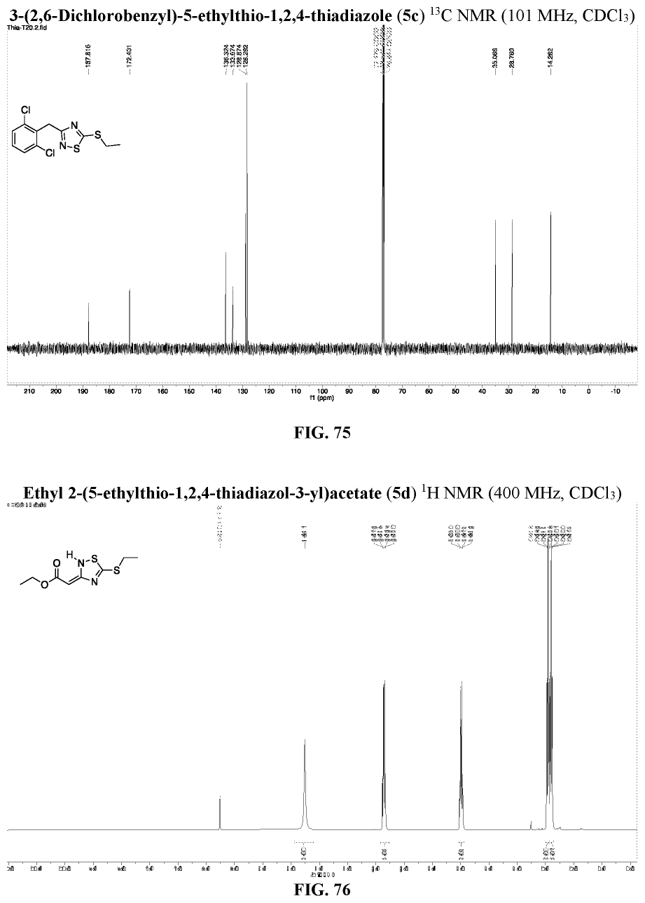

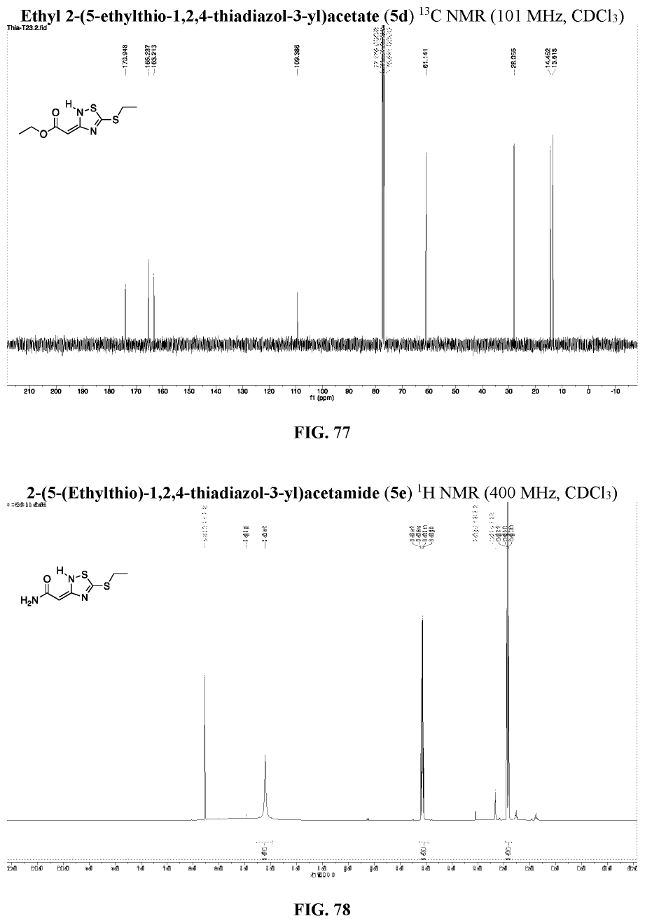

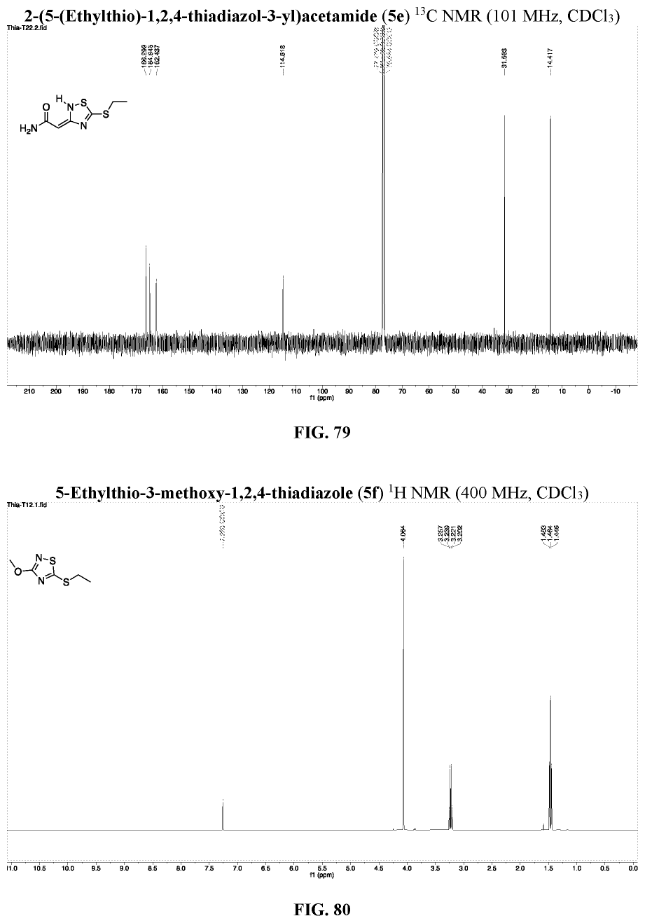

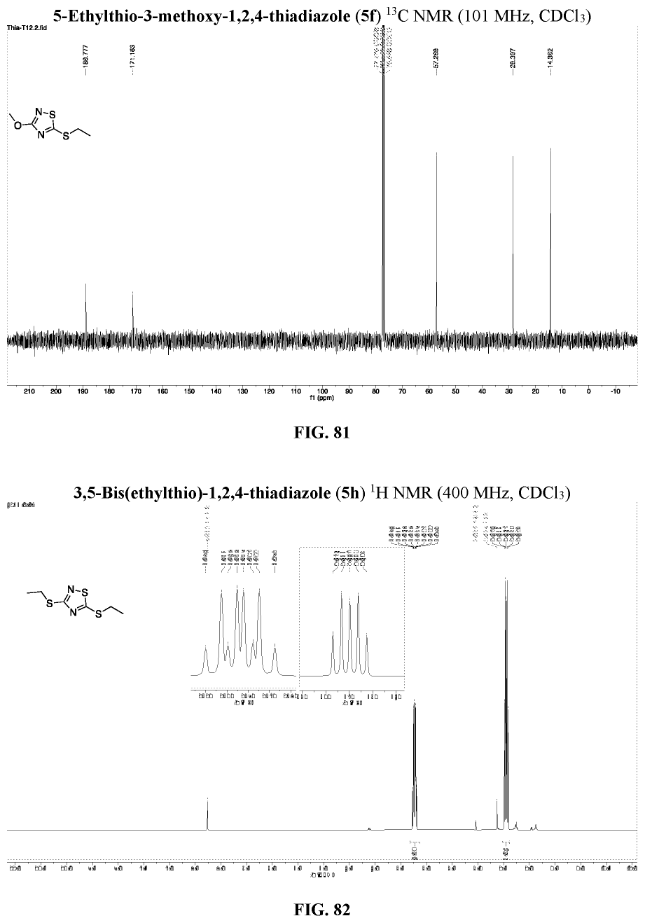

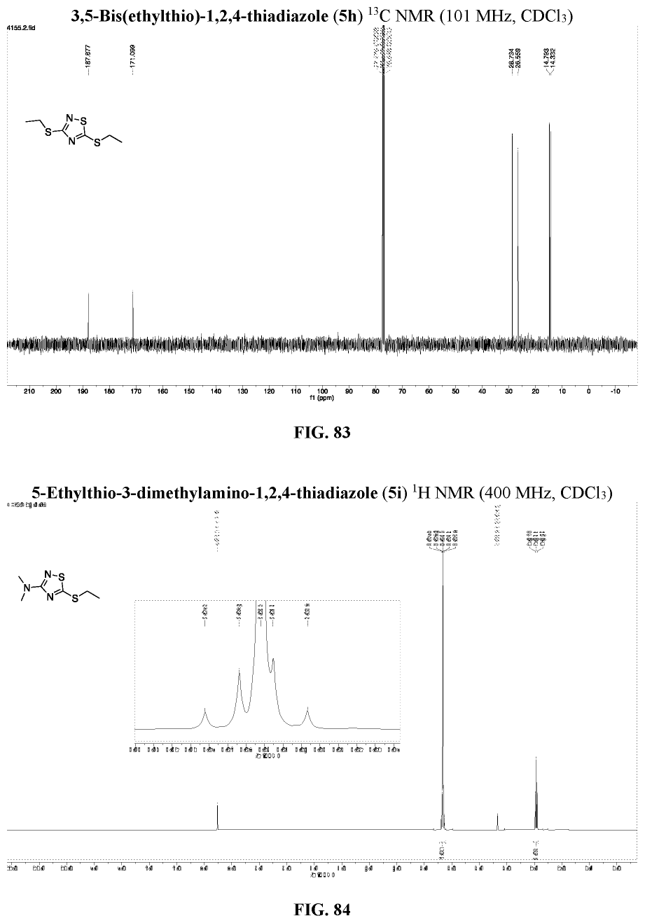

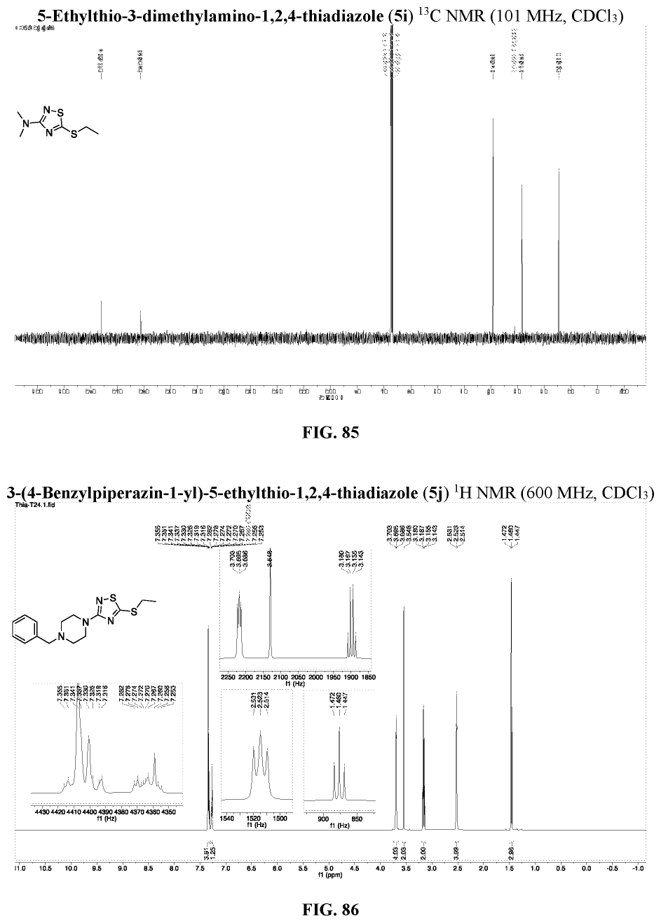

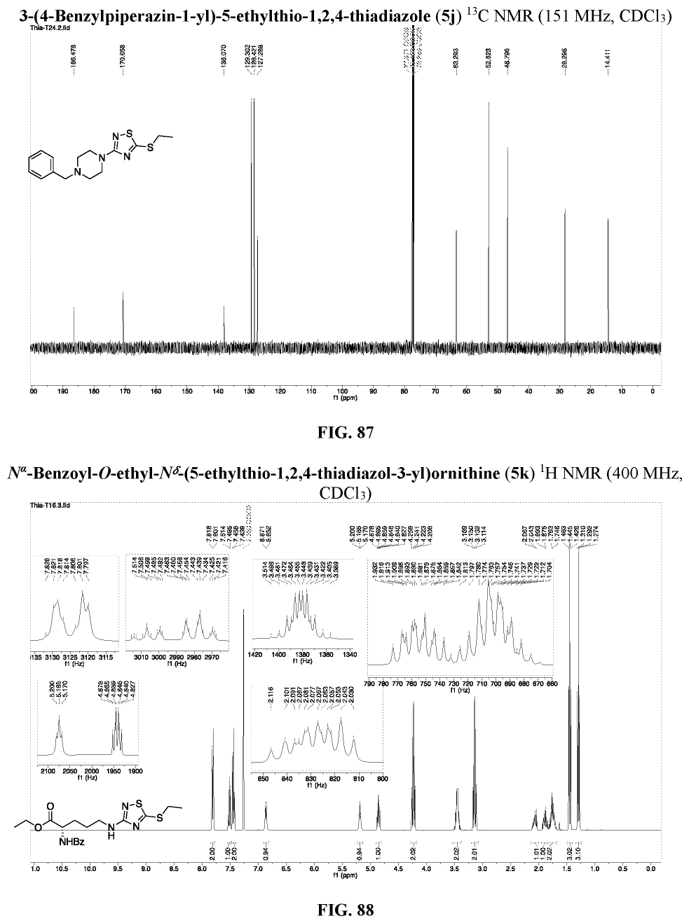

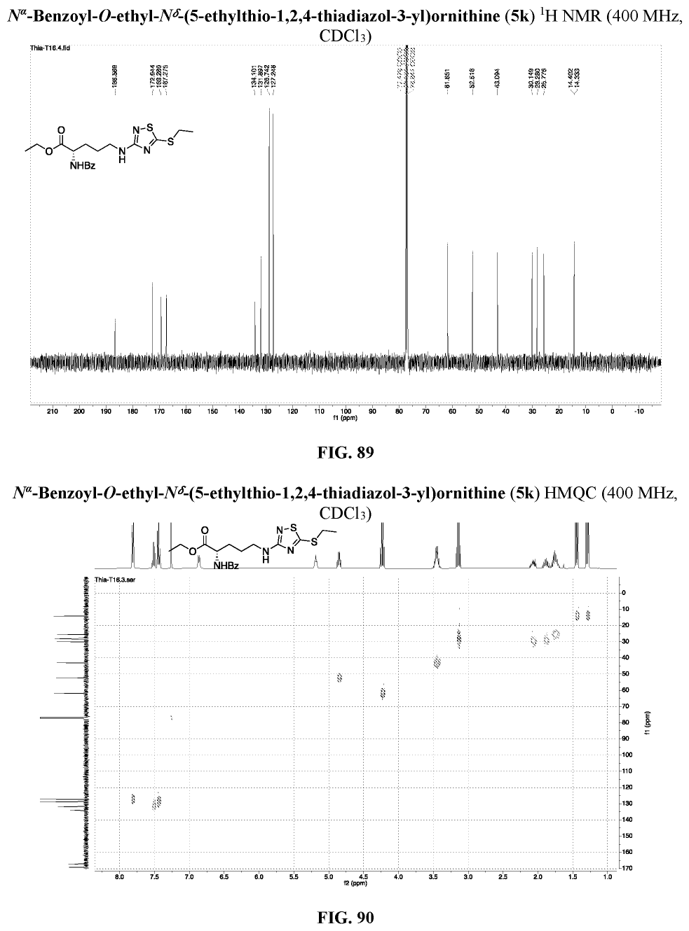

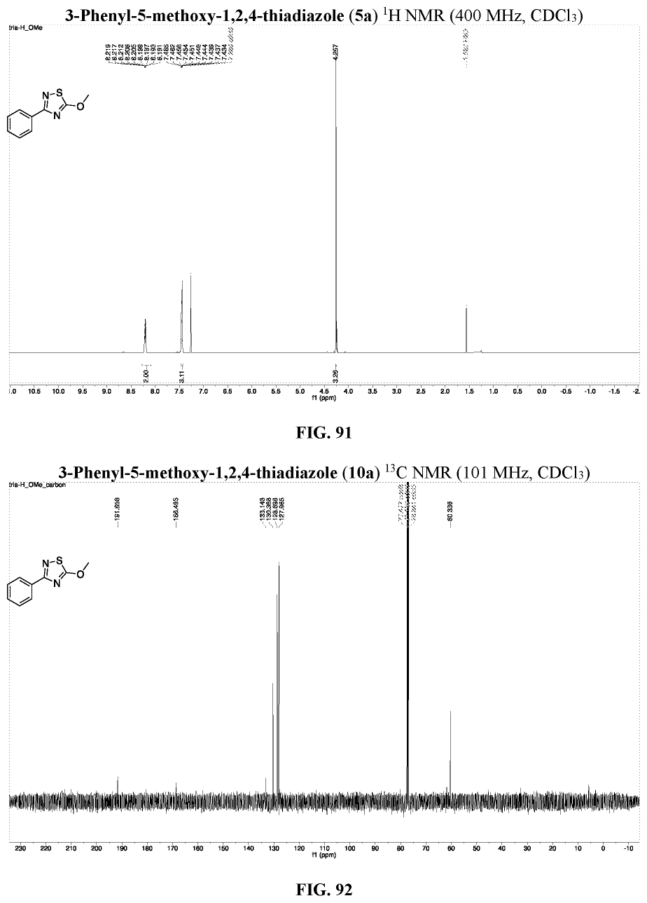

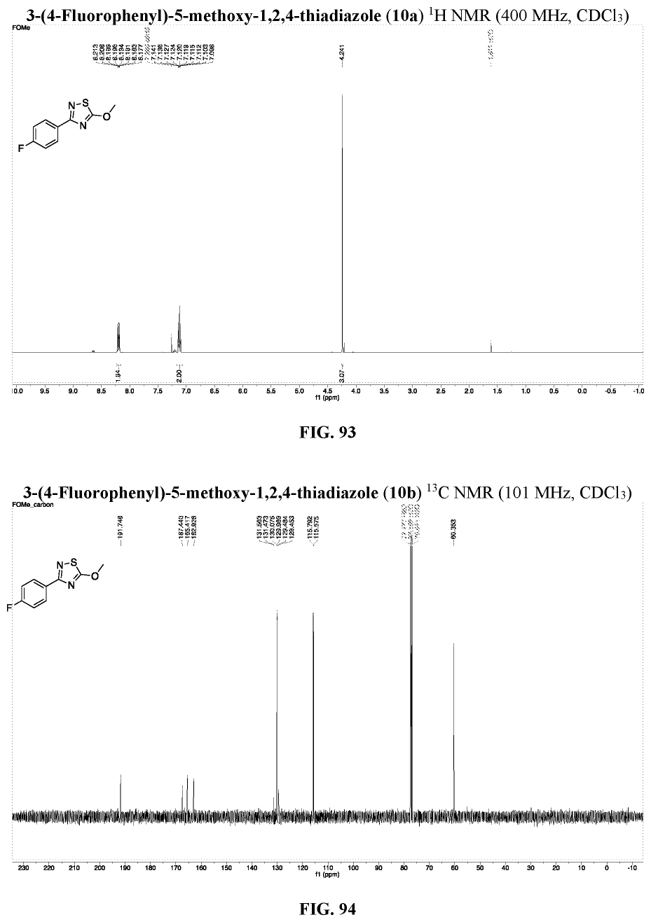

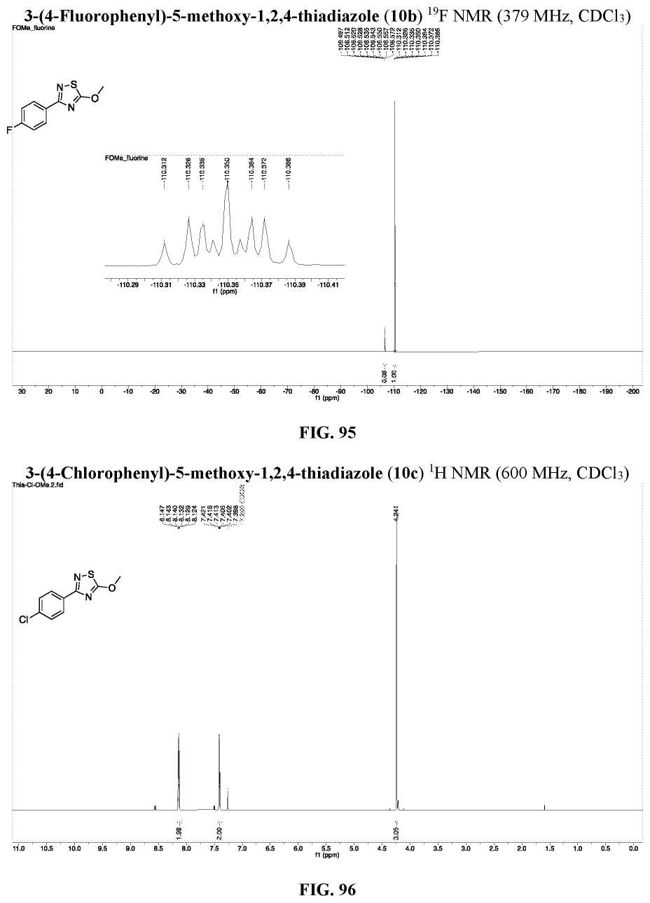

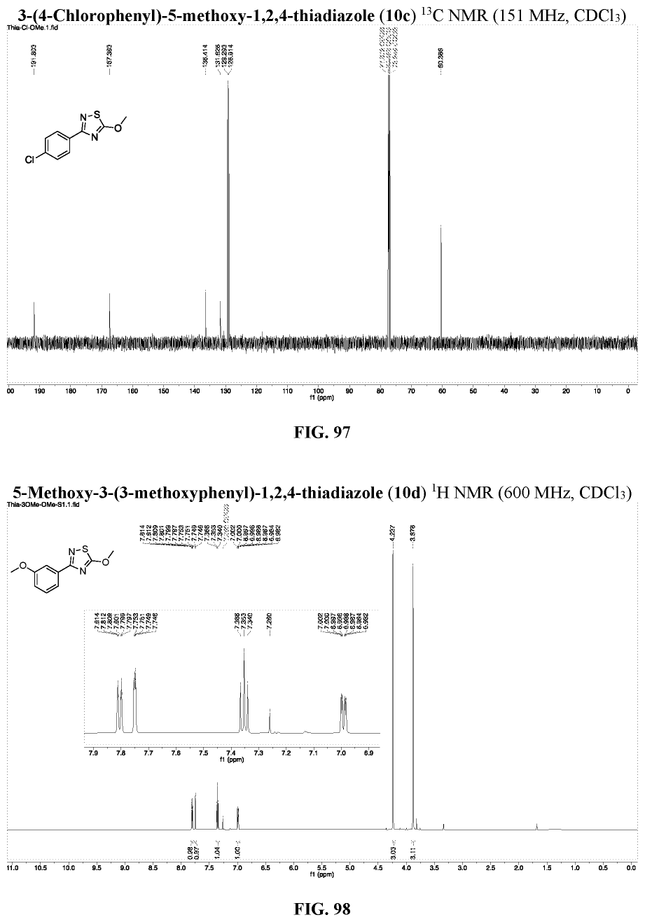

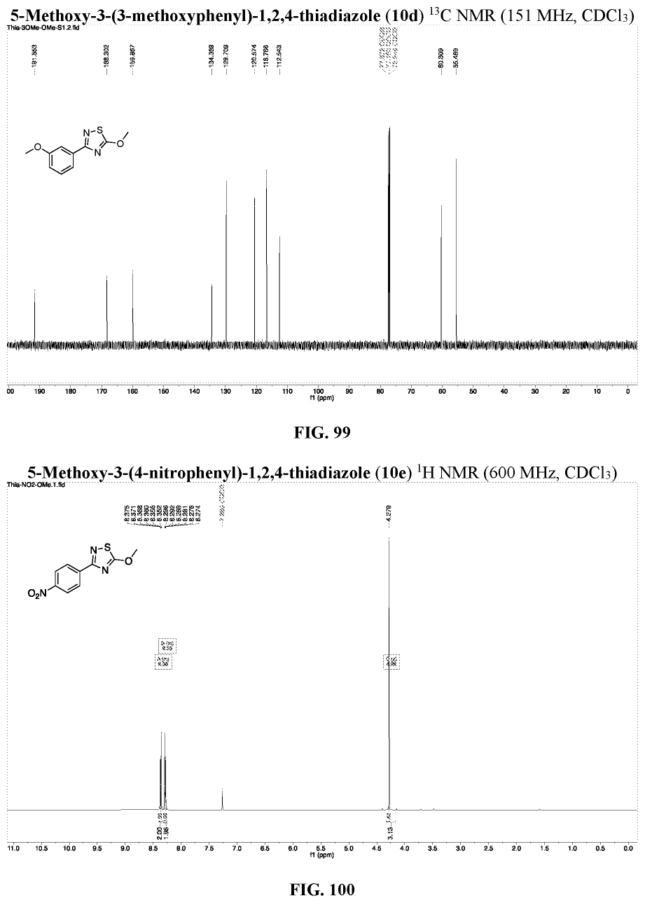

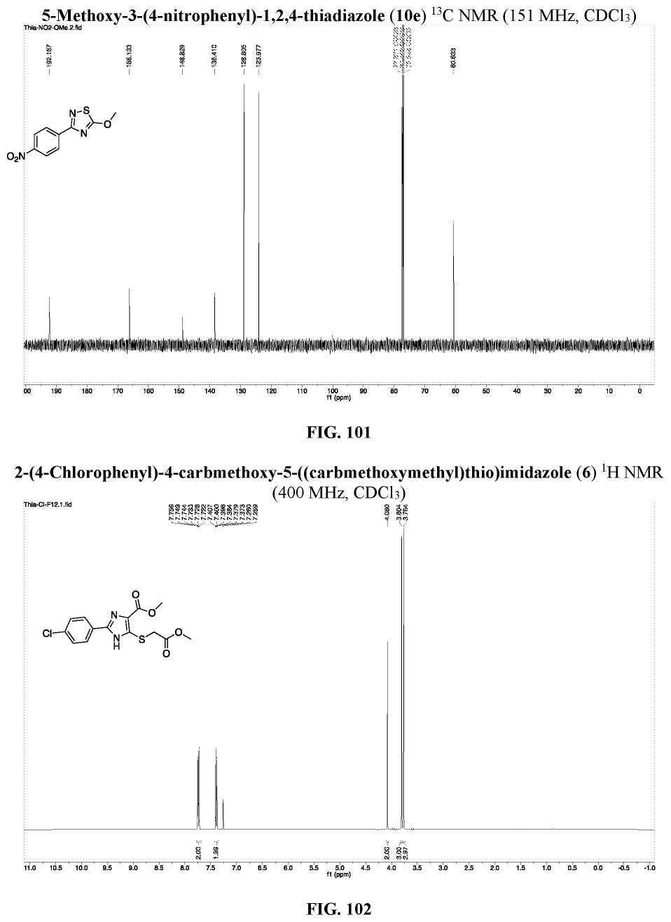

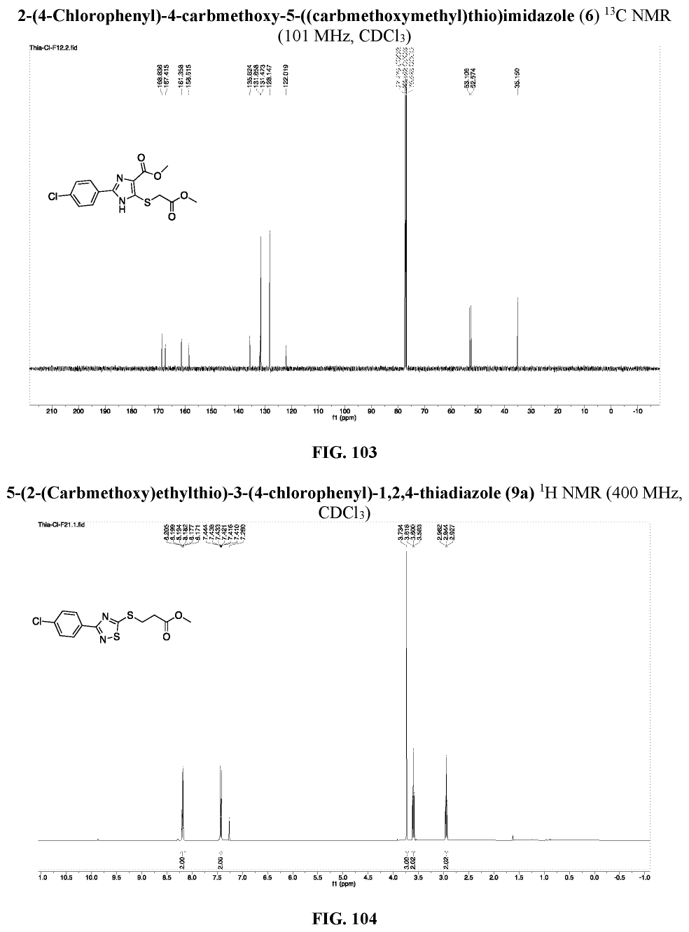

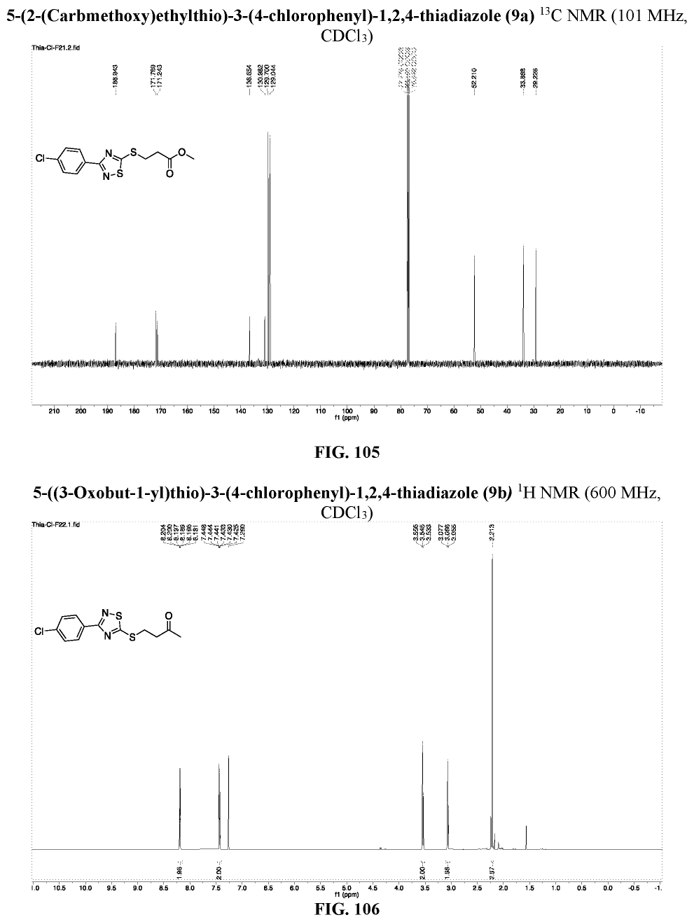

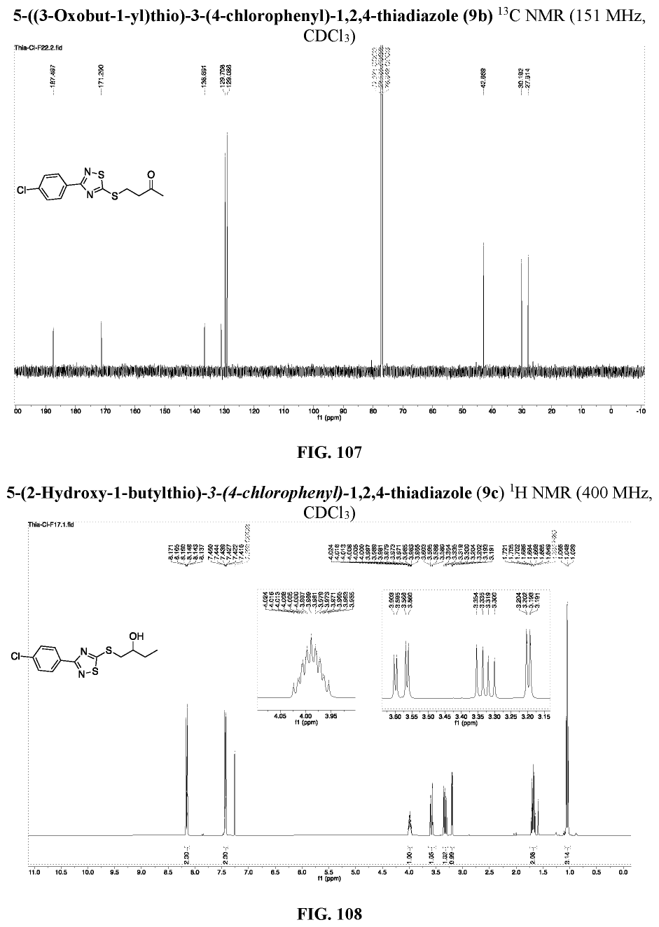

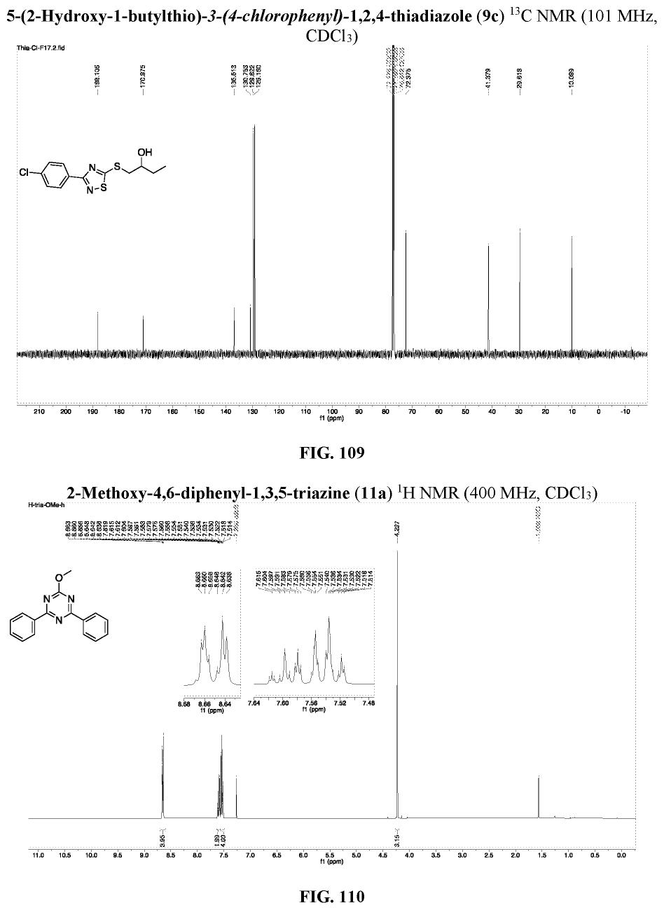

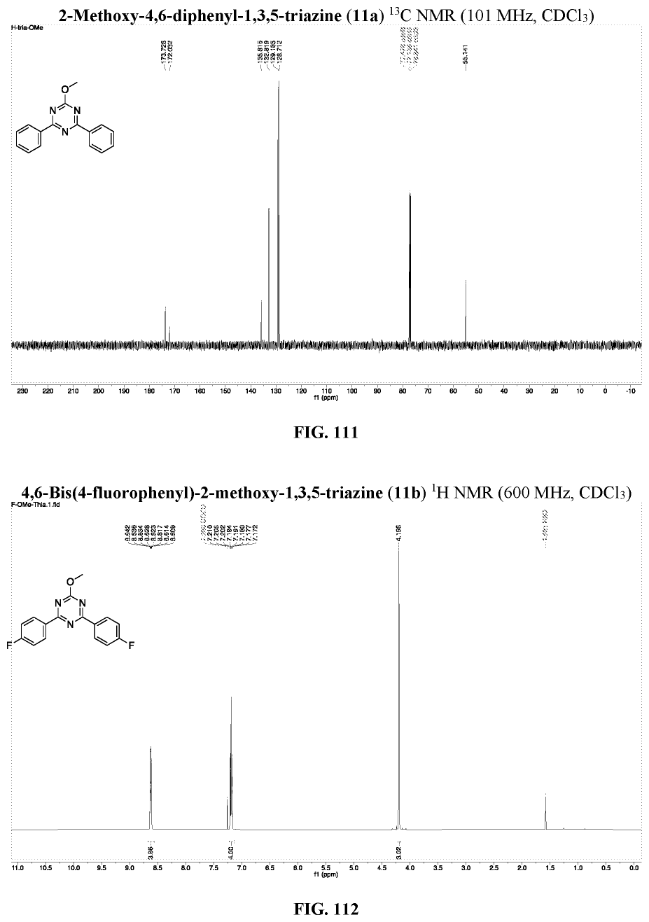

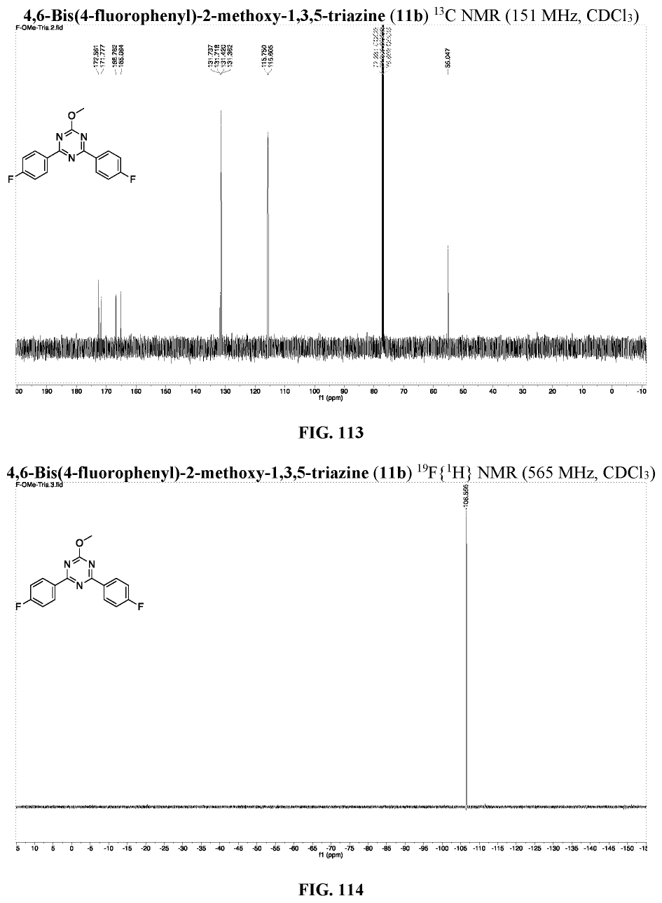

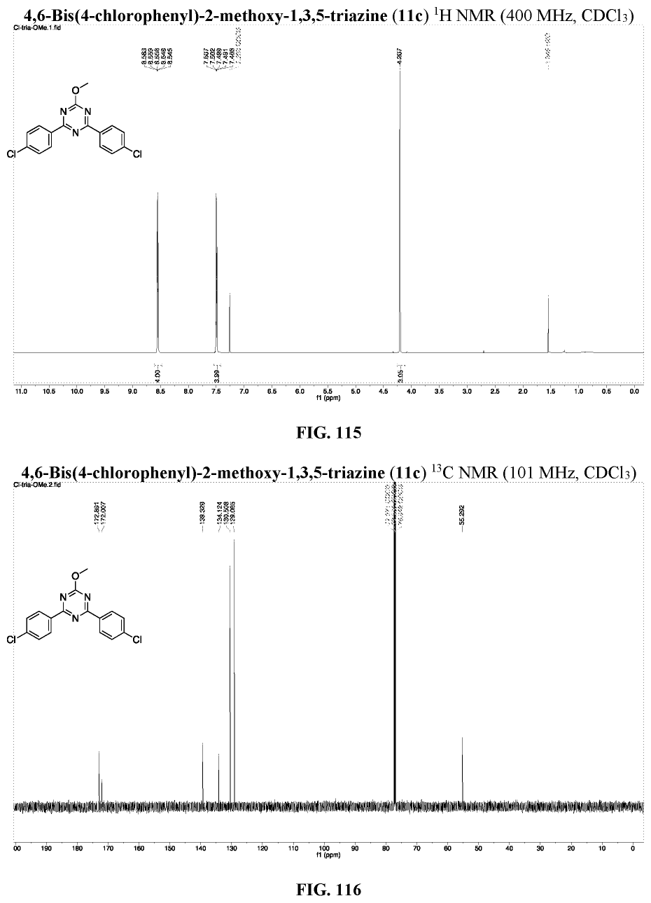

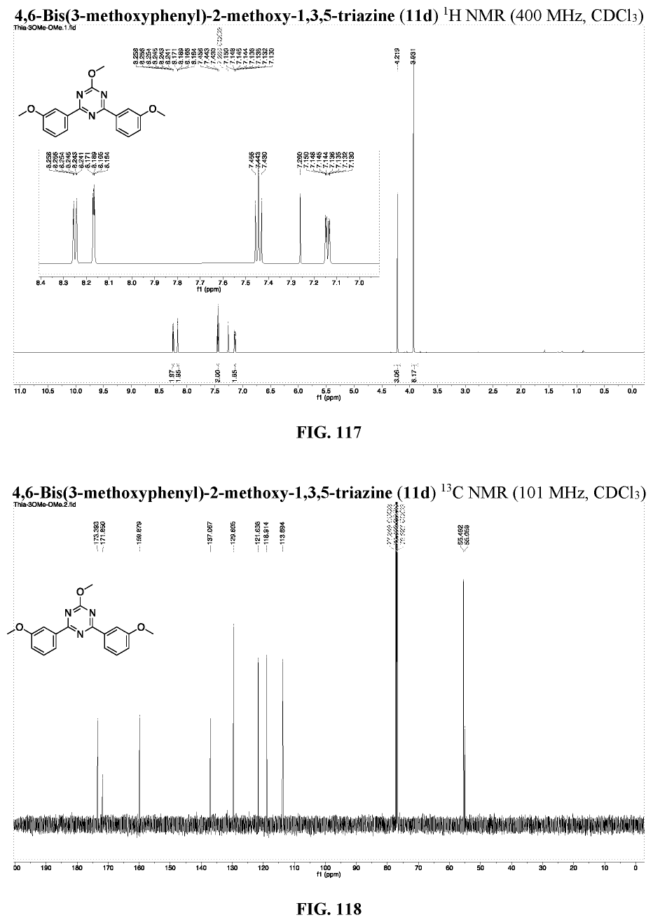

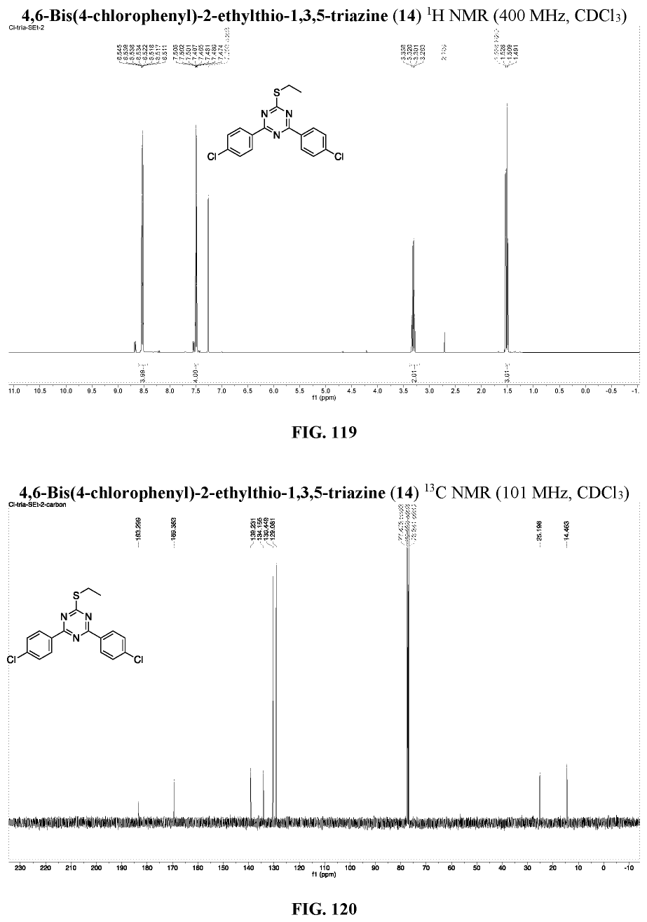

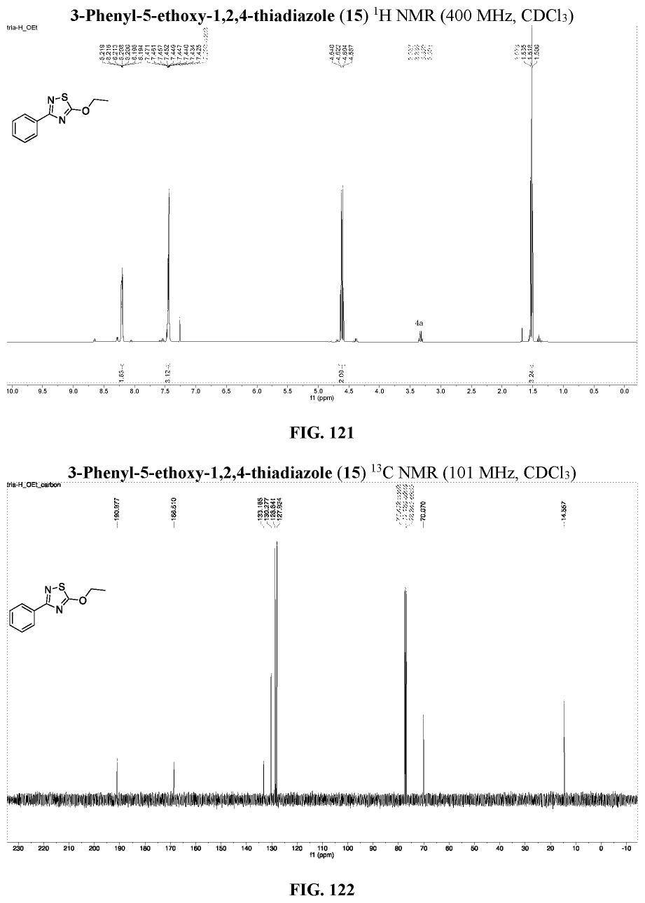

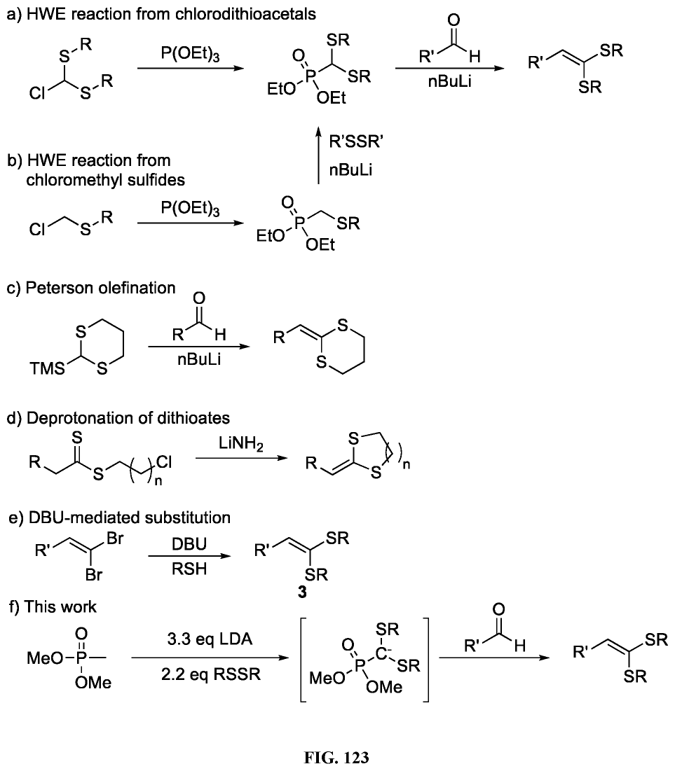

FIG. 8 shows the proposed route to the products 10 and 11 from the corresponding amidine. Briefly, the N,N′-bis(iminomethyl)thiourea 12 is produced by the addition of an equivalent of amidine to 3, accompanied by the loss of ethanethiol. In an intramolecular addition, similar to that of the Pinner triazole synthesis (Schaefer, F. C., “Synthesis of the s-triazine System. VI. 1 Preparation of Unsymmetrically Substituted s-triazines by Reaction of Amidine Salts With Imidates,” J. Org. Chem. 27:3608-3613 (1962); Pinner & Ueber, “Diphenyloxykyanidin,” Chem. Ber. 23:2919 (1890), which are hereby incorporated by reference in their entirety), 12 cyclizes to form 1,3,5-triazine-2(5H)-thione 13 after the loss of ammonia. In the presence of ethyl bromide and DBU, 13 is S-alkylated to form 14, which, in the presence of DBU and methanol, undergoes nucleophilic displacement to yield the methoxy-substituted triazine 11. Compound 14 was not detected when the reaction was run in methanol, suggesting that the substitution reaction occurs rapidly; however, when the reaction was performed in acetonitrile at reflux instead of methanol, 14 was isolated in 34% yield. When synthesis of higher analogs of 10 was attempted by performing the reaction in ethanol or isopropanol in lieu of methanol, no thiadiazole or triazine was detected. However, by simply changing the solvent from neat ethanol to a 1:1 mix of acetonitrile and ethanol allowed production of 5-ethoxy-3-phenyl-1,2,4-thiadiazole (15) in 24% yield (FIG. 9).

Conclusions

In summary, a versatile synthesis of 5-alkylthio-1,2,4-thiadiazoles has been developed from amidines and alkyl halides using carbon disulfide as a carbon and sulfur source, and N-chlorosuccinimide as an easily-accessible oxidant. The reaction works with alkyl, aryl, and heteroaryl amidines, as well as N-substituted guanidines and O-substituted isoureas, while using S-substituted isothioureas results in a mixture of products due to scrambling of the alkylthio groups. Although this synthesis of 5-alkylthio-1,2,4-thiadiazoles is somewhat narrowed when using alkyl halides on the basis of steric demands other nucleophiles like methyl acrylate and 1,2-epoxybutane also react with the intermediate amidine-carbon disulfide adduct, permitting the formation of diverse thiadiazoles. Additionally, by changing the reaction conditions, several other products could be obtained, including either 5-methoxy-1,2,4-thiadiazoles or 2-methoxy-1,3,5-triazines when performing the reaction in methanol. Given that 1,2,4-thiadiazoles have traditionally been underrepresented in leads for biologically active compounds, it is hoped that that this new, one-pot synthesis of 5-alkylthio-1,2,4-diazoles will improve the availability of these compounds for future exploration.

### Experimental

General Information

All solvents were purchased from Fisher Scientific and used as received. Potassium carbonate was ground in a mortar and pestle and oven-dried at 250° C. for 24 hours before use. All amidine salts were purchased from TCI, Alfa Aesar, Oakwood Chemicals, Chem-Impex, or Maybridge and were used as received. Reaction products were visualized via TLC under UV light or by staining with KMnO₄ or cerium ammonium molybdenate stain. 5-Alkylthio-1,2,4-thiadiazoles were purified on a Buchi Pure C-810 Flash chromatography system using HPLC grade solvents on 12 g or 25 g FlashPure silica cartridges, except where noted. The characterization of all compounds was performed at the Iowa State University Chemical Instrumentation Facility. NMR spectra were obtained using Avance NEO 400 MHz and Avance III 600 MHz spectrometers. Chemical shifts are reported in ppm relative to the residual solvent peak (CDCl₃: 7.26 ppm for ¹H and 77.16 ppm for ¹³C; DMSO-d₆: 2.50 for ¹H and 39.52 ppm for ¹³C) (Fulmer et al., “NMR Chemical Shifts of Trace Impurities: Common Laboratory Solvents, Organics, and Gases in Deuterated Solvents Relevant to the Organometallic Chemist,” Organometallics 29:2176-2179 (2010), which is hereby incorporated by reference in its entirety). Coupling constants are reported in Hz. HRMS analysis was performed using positive ion mode mass spectra on an Agilent QTOF 6540 mass spectrometer. Accurate mass measurement was achieved by constantly infusing a calibrant (masses: 121.0508 and 922.0098). Melting points are uncorrected and were obtained on Stuart SMP30 melting point apparatus using a temperature ramp rate of not greater than 5° C. min⁻¹.

Ethyl 1-imidazolecarbodithioate (2) Modifying a literature procedure (Sun et al., “1-(Methyldithiocarbonyl)imidazole: A reagent of S-methyldithiocarbonylation,” Synlett 1997:1279-1280 (1997), which is hereby incorporated by reference in its entirety), sodium hydride (60% dispersion in oil, 0.504 g, 1.05 eq) was suspended in dry THE (50 mL) under an argon atmosphere, and imidazole (1.362 g, 20 mmol) was added portionwise over five minutes at 0° C. The suspension was stirred at this temperature for 15 minutes, during which time a heavy precipitate formed. Carbon disulfide (1.675 g, 1.1 eq) was added over 3 minutes at 0° C., and the solution became clear and deep orange. After 30 min at 0° C., bromoethane (2.397 g, 1.1 eq) was added in one portion, and the reaction was warmed to room temperature and stirred for 1 hour. The solution was then reduced under vacuum to a volume of approximate 20 mL, and ethyl acetate (50 mL) and water (20 mL) were added. The aqueous layer was removed, and the organic layer was washed again with water (20 mL) and brine (20 mL), and then dried over anhydrous magnesium sulfate. The solvent was removed, and the crude material was purified by flash chromatography (80:20 hexane:ethyl acetate) to yield a bright yellow oil (2.69 g, 78%). ¹H NMR (400 MHz, CDCl₃) δ 8.47 (t, J=1.0 Hz, 1H), 7.77 (t, J=1.5 Hz, 1H), 7.09 (dd, J=1.8, 0.9 Hz, 1H), 3.39 (q, J=7.4 Hz, 2H), 1.43 (t, J=7.4 Hz, 3H). ¹³C NMR (101 MHz, CDCl₃) δ 198.2, 135.7, 131.4, 117.8, 31.4, 12.6.

S-Ethyl-N-(α-iminobenzyl)dithiocarbamate (3a) From 2: Benzamidine hydrochloride (157 mg, 1 mmol) and powdered anhydrous potassium carbonate (346 mg, 2.5 eq) were added to acetonitrile (5 mL), followed by the addition of 2 (181 mg, 1.05 eq). The reaction was heated to 50° C. for 18 hours, and then cooled to 22° C. The reaction was then poured into water (25 mL), and extracted twice with ethyl acetate (20 mL). The combined organic layers were washed twice with water (20 mL) and brine (20 mL), and dried over anhydrous magnesium sulfate. The solvent was removed under vacuum, and the crude deep orange liquid was purified by column chromatography (eluent: 90:10 to 60:40 hexane:ethyl acetate) to yield the title compound as a viscous orange liquid (190 mg, 85% yield).

From carbon disulfide and bromoethane: Benzamidine hydrochloride (157 mg, 1 mmol) and DBU (167 mg, 1.1 mmol) were dissolved in acetonitrile (10 mL) at 22° C., and carbon disulfide (114 mg, 1.1 mmol) and bromoethane (131 mg) were added in one portion. The reaction was allowed to stir 18 hours, and the reaction was then worked up as above. ¹H NMR (400 MHz, CDCl₃) δ 11.4 (broad s, 1H), 7.97-7.90 (m, 2H), 7.62-7.54 (m, 1H), 7.53-7.46 (m, 2H), 7.0 (broad s, 1H), 3.21 (q, J=7.4 Hz, 2H), 1.38 (t, J=7.4 Hz, 3H). ¹H NMR (400 MHz, DMSO-d₆) δ 10.59 (broad s, 1H), 9.68 (broad s, 1H), 8.06-7.98 (m, 2H), 7.68-7.59 (m, 1H), 7.59-7.51 (m, 1H), 3.10 (q, J=7.3 Hz, 2H), 1.27 (t, J=7.4 Hz, 3H). ¹³C NMR (101 MHz, CDCl₃) δ 217.4, 161.9, 134.0, 132.9, 129.0, 127.7, 29.8, 13.4. ¹³C NMR (101 MHz, DMSO-d₆) δ 212.8, 161.7, 133.5, 132.7, 128.7, 128.2, 28.8, 14.0. HRMS (+ESI-QTOF) m/z: [M+H]⁺ calcd for C₁₀H₁₃N₂S₂⁺ 225.0515; found: 225.0512.

S-Ethyl-N-(α-imino-4-chlorobenzyl)dithiocarbamate (3c) Collected as an impurity during the synthesis of 14. Yellow-orange crystalline solid. mp 116-118° C. ¹H NMR (600 MHz, CDCl₃) δ 11.3 (broad s, 1H), 7.88-7.82 (m, 2H), 7.46-7.41 (m, 2H), 6.8 (broad s, 1H), h 3.18 (q, J=7.4 Hz, 2H), 1.36 (t, J=7.4 Hz, 3H). ¹³C NMR (151 MHz, CDCl₃) δ 218.5, 160.4, 139.1, 133.0, 129.3, 129.0, 29.8, 13.5. HRMS (+ESI-QTOF) m/z: [M+H]⁺ calcd for C₁₀H₁₂ClN₂S₂⁺ 259.0125; found: 259.0127.

### General Procedure for the Synthesis of 5-alkylthio-1,2,4-thiadiazoles (GP1)

Amidine hydrochloride (1.0 mmol) and DBU (312 mg, 2.05 mmol) were added to acetonitrile (10 mL), followed by the addition of carbon disulfide (84 mg, 1.1 mmol) and alkyl halide (1.2 mmol), and the reaction was stirred at 40° C. for 18 hours. The deep yellow-to-red reaction mixture was then cooled to 0° C., and NCS (147 mg, 1.1 eq) was added in a single portion. The reaction was stirred at 22° C. and 1,2,4-thiadiazole formation was monitored by TLC, and upon consumption of the S-alkyl (iminomethyl)dithiocarbamate (typically 15-60 minutes), excess NCS was quenched by the addition of 1 M sodium thiosulfate solution (2 mL). The reaction was poured into water (15 mL), and the biphasic mixture was extracted with hexane (2×10 mL). The organic layer was washed with water (2×10 ml), then 1 M hydrochloric acid (10 mL), 1 M sodium hydroxide (10 mL), and brine (10 mL), and dried over anhydrous sodium sulfate. The solvent was removed under vacuum, and the crude product was purified by flash chromatography on silica using a gradient of 100:0 to 90:10 hexane:ethyl acetate as an eluent.

5-Ethylthio-3-phenyl-1,2,4-thiadiazole (Ginsberg & Goerdeler, “Über 1.2.4-Thiodiazole, XIV. Thiodiazol-3- und 5-diazoniumsalze,” Chem. Ber. 94:2043-2060 (1961), which is hereby incorporated by reference in its entirety) (4a). From 3a: To acetonitrile (5 mL) was added 3aa (224 mg, 1 mmol) and DBU (160 mg, 1.05 eq). The solution was cooled to 0° C., and NCS (147 mg, 1.1 eq) was added. After 30 minutes, the reaction was worked up as in GP1.

From 1a, Method A: Synthesized according to GP1 from benzamidine hydrochloride (157 mg) and bromoethane (131 mg). Colorless liquid that solidifies to a white, crystalline solid upon cooling (163 mg, 73% yield). From 1a, Method B: Iodoethane (187 mg) used instead of bromoethane. Yield: 165 mg (75%). mp 37-39° C. (lit.⁴² 37-38° C.). ¹H NMR (400 MHz, CDCl₃) δ 8.31-8.25 (m, 2H), 7.51-7.43 (m, 3H), 3.34 (q, J=7.4 Hz, 2H), 1.54 (t, J=7.4 Hz, 3H). ¹³C NMR (101 MHz, CDCl₃) δ 187.6, 172.6, 132.7, 130.5, 128.8, 128.4, 28.8, 14.4. HRMS (+ESI-QTOF) m/z: [M+H]⁺ calcd for C₁₀H₁₁N₂S₂⁺ 223.0358; found: 223.0357.

5-Methylthio-3-phenyl-1,2,4-thiadiazole (U.S. Pat. No. 3,770,754 to Parsons, which is hereby incorporated by reference in its entirety) (4b). Synthesized according to GP1 from benzamidine hydrochloride (157 mg) and iodomethane (170 mg). Eluent: 100:0 to 90:10 hexane:ethyl acetate. White, crystalline solid (153 mg, 69% yield). mp 81-82° C. (lit. (U.S. Pat. No. 3,770,754 to Parsons, which is hereby incorporated by reference in its entirety) 76° C.). ¹H NMR (400 MHz, CDCl₃) δ 8.31-8.25 (m, 2H), 7.50-7.44 (m, 3H), 2.80 (s, 3H). ¹³C NMR (101 MHz, CDCl₃) δ 188.7, 172.7, 132.6, 130.5, 128.8, 128.4, 16.7. HRMS (+ESI-QTOF) m/z: [M+H]⁺ calcd for C₉H₉N₂S₂⁺ 209.0202; found: 209.0198.

3-Phenyl-5-propylthio-1,2,4-thiadiazole (4c) Synthesized according to GP1 from benzamidine hydrochloride (157 mg) and 1-bromopropane (148 mg). Colorless liquid (162 mg, 68% yield). ¹H NMR (400 MHz, CDCl₃) δ 8.32-8.22 (m, 2H), 7.54-7.41 (m, 3H), 3.30 (t, J=7.2 Hz, 2H), 1.91 (h, J=7.3 Hz, 2H), 1.12 (t, J=7.3 Hz, 3H). ¹³C NMR (101 MHz, CDCl₃) δ 188.0, 172.6, 132.7, 130.5, 128.8, 128.4, 36.5, 22.6, 13.5. HRMS (+ESI-QTOF) m/z: [M+H]⁺ calcd for C₁₁H₁₃N₂S₂⁺ 237.0515; found: 237.0518.

5-Isopropylthio-3-phenyl-1,2,4-thiadiazole (4d) Synthesized according to GP1 from benzamidine hydrochloride (157 mg) and 2-iodopropane (204 mg). Colorless oil (137 g, 51%). ¹H NMR (400 MHz, CDCl₃) δ 8.31-8.24 (m, 2H), 7.50-7.44 (m, 3H), 4.00 (hept, J=6.8 Hz, 1H), 1.56 (d, J=6.8 Hz, 6H). ¹³C NMR (101 MHz, CDCl₃) δ 187.0, 172.5, 132.7, 130.5, 128.8, 128.4, 40.5, 23.3. HRMS (+ESI-QTOF) m/z: [M+H]⁺ calcd for C₁₁H₁₃N₂S₂⁺ 237.0515; found: 237.0514.

5-Isobutylthio-3-phenyl-1,2,4-thiadiazole (4e) Synthesized according to GP1 from benzamidine hydrochloride (157 mg) and 1-bromo-2-methylpropane (164 mg). Colorless liquid (109 mg, 44% yield). ¹H NMR (400 MHz, CDCl₃) δ 8.32-8.22 (m, 1H), 7.54-7.41 (m, 2H), 3.22 (d, J=6.8 Hz, 1H), 2.14 (dh, J=6.8, 6.6 Hz, 1H), 1.12 (d, J=6.6 Hz, 3H). ¹³C NMR (101 MHz, CDCl₃) δ 188.3, 172.5, 132.7, 130.5, 128.8, 128.4, 43.1, 28.7, 22.1. HRMS (+ESI-QTOF) m/z: [M+H]⁺ calcd for C₁₂H₁₅N₂S₂⁺ 251.0671; found: 251.0673.

5-(Cyclopropylmethylthio)-3-phenyl-1,2,4-thiadiazole (4f) Synthesized according to GP1 from benzamidine hydrochloride (157 mg) and cyclopropylmethyl bromide (162 mg). Colorless liquid (169 mg, 68% yield). ¹H NMR (400 MHz, CDCl₃) δ 8.33-8.22 (m, 1H), 7.52-7.41 (m, 2H), 3.29 (d, J=7.3 Hz, 1H), 1.29 (ttt, J=8.0, 7.3, 4.7 Hz 1H), 0.79-0.62 (m, 1H), 0.48-0.37 (m, 1H). ¹³C NMR (101 MHz, CDCl₃) δ 187.8, 172.5, 132.7, 130.5, 128.8, 128.4, 40.4, 10.5, 6.3. HRMS (+ESI-QTOF) m/z: [M+H]⁺ calcd for C₁₂H₁₃N₂S₂⁺ 249.0515; found: 249.0514.

5-Allylthio-3-phenyl-1,2,4-thiadiazole (U.S. Pat. No. 3,770,754 to Parsons, which is hereby incorporated by reference in its entirety) (4g) Synthesized according to GP1 from benzamidine hydrochloride (157 mg) and allyl bromide (145 mg). Colorless liquid (173 mg, 74% yield). ¹H NMR (400 MHz, CDCl₃) δ 8.32-8.22 (m, 2H), 7.51-7.41 (m, 3H), 6.02 (ddt, J=16.9, 10.0, 6.9 Hz, 1H), 5.45 (dq, J=16.9, 1.3 Hz, 1H), 5.28 (dq, J=10.0, 1.0 Hz, 1H), 3.97 (dt, J=6.9, 1.2 Hz, 2H). ¹³C NMR (101 MHz, CDCl₃) δ 186.7, 172.4, 132.6, 131.7, 130.5, 128.8, 128.4, 120.1, 37.0. HRMS (+ESI-QTOF) m/z: [M+H]⁺ calcd for C₁₁H₁₁N₂S₂⁺ 235.0358; found: 235.0361.

5-Benzylthio-3-phenyl-1,2,4-thiadiazole (Park et al., “Parallel Synthesis of Drug-like 5-amino-substituted 1,2,4-thiadiazole Libraries Using Cyclization Reactions of a Carboxamidine Dithiocarbamate Linker,” Synthesis 2009:913-920 (2009), which is hereby incorporated by reference in its entirety) (4h) Synthesized according to GP1 from benzamidine hydrochloride (157 mg) and benzyl bromide (205 mg). Viscous, colorless oil (198 mg, 70% yield); lit. (Park et al., “Parallel Synthesis of Drug-like 5-amino-substituted 1,2,4-thiadiazole Libraries Using Cyclization Reactions of a Carboxamidine Dithiocarbamate Linker,” Synthesis 2009:913-920 (2009), which is hereby incorporated by reference in its entirety) white solid; no mp given. ¹H NMR (400 MHz, CDCl₃) δ 8.33-8.26 (m, 2H), 7.53-7.43 (m, 5H), 7.39-7.28 (m, 3H), 4.57 (s, 2H). ¹³C NMR (101 MHz, CDCl₃) δ 186.8, 172.3, 135.6, 132.6, 130.5, 129.2, 129.0, 128.8, 128.4, 128.2, 38.6. HRMS (+ESI-QTOF) m/z: [M+H]⁺ calcd for C₁₅H₁₃N₂S₂⁺ 285.0515; found: 285.0515.

5-Citronellylthio-3-phenyl-1,2,4-thiadiazole (4i) Synthesized according to GP1 from benzamidine hydrochloride (157 mg) and racemic citronellyl bromide (263 mg). Eluent: 100:0 to 93:7 hexane:ethyl acetate. Colorless oil (201 mg, 60% yield). ¹H NMR (600 MHz, CDCl₃) δ 8.31-8.24 (m, 2H), 7.50-7.43 (m, 3H), 5.10 (tp, J=7.1, 1.4 Hz, 1H), 3.36 (ddd, J=12.6, 9.6, 5.3 Hz, 1H), 3.29 (ddd, J=12.6, 9.2, 6.3 Hz, 1H), 2.04 (dp, J=14.6, 7.2 Hz, 2H), 1.99 (dp, J=14.6, 7.2 Hz, 2H), 1.89 (dddd, J=12.7, 9.2, 6.2, 4.8 Hz, 1H), 1.75-1.62 (m, 5H), 1.60 (d, J=1.3 Hz, 3H), 1.46-1.38 (m, 1H), 1.25 (dddd, J=13.3, 9.5, 7.5, 5.9 Hz, 1H), 1.00 (d, J=6.5 Hz, 3H). ¹³C NMR (151 MHz, CDCl₃) δ 187.9, 172.6, 132.7, 131.7, 130.5, 128.8, 128.4, 124.5, 36.8, 36.1, 32.4, 32.1, 25.9, 25.6, 19.4, 17.8. HRMS (+ESI-QTOF) m/z: [M+H]⁺ calcd for C₁₈H₂₅N₂S₂⁺ 333.1454; found: 333.1457.

5-Ethylthio-3-(4-fluorophenyl)-1,2,4-thiadiazole (4j) Synthesized according to the general procedure from 4-fluorobenzamidine hydrochloride (175 mg) and bromoethane (131 mg). White, crystalline solid (177 mg, 74% yield). mp 35-36° C. ¹H NMR (400 MHz, CDCl₃) δ 8.29-8.24 (m, J_{HF}=11.4, 2H), 7.17-7.11 (m, J_{HF}=8.8, 2H), 3.32 (q, J=7.4 Hz, 2H), 1.53 (t, J=7.4 Hz, 3H). ¹³C NMR (101 MHz, CDCl₃) δ 187.9, 171.5, 164.3 (d, J_{CF}=250.5 Hz), 130.5 (d, J_{CF}=8.7 Hz), 129.0 (d, J_{CF}=3.2 Hz), 115.8 (d, J_{CF}=21.9 Hz), 28.8, 14.4. ¹⁹F NMR (376 MHz, CDCl₃) δ −110.06 (tt, J=8.6, 5.3 Hz). HRMS (+ESI-QTOF) m/z: [M+H]⁺ calcd for C₁₀H₁₀FN₂S₂⁺ 241.0264; found: 241.0260.

3-(4-Chlorophenyl)-5-ethylthio-1,2,4-thiadiazole (4k) Synthesized according to the general procedure from 4-chlorobenzamidine hydrochloride (191 mg) and bromoethane (131 mg). White crystalline solid (188 mg, 73% yield). mp 44-45° C. ¹H NMR (400 MHz, CDCl₃) δ 8.24-8.17 (m, 2H), 7.48-7.40 (m, 2H), 3.33 (q, J=7.4 Hz, 2H), 1.54 (t, J=7.4 Hz, 3H). ¹³C NMR (101 MHz, CDCl₃) δ 188.1, 171.5, 136.6, 131.2, 129.7, 129.0, 28.9, 14.4. HRMS (+ESI-QTOF) m/z: [M+H]⁺ calcd for C₁₀H₁₀ClN₂S₂⁺ 256.9968; found: 256.9970.

3-(4-Chlorophenyl)-5-methylthio-1,2,4-thiadiazole (4l) Performed on 5 mmol scale from 4-chlorobenzamidine hydrochloride (955 mg, 5 mmol) and iodomethane (681 mg, 6.25 mmol, 1.2 eq). Beige semi-crystalline solid, which may be recrystallized from hot heptane to give white needles (846 mg, 70% yield). mp 105-107° C. (lit.⁴³ 101° C.). ¹H NMR (400 MHz, CDCl₃) δ 8.24-8.17 (m, 2H), 7.46-7.40 (m, 2H), 2.79 (s, 3H). ¹³C NMR (101 MHz, CDCl₃) δ 189.12, 171.63, 136.61, 131.10, 129.73, 129.02, 16.70. HRMS (+ESI-QTOF) m/z: [M+H]⁺ calcd for C₉H₈ClN₂S₂⁺ 242.9812; found: 242.9810.

3-(4-chlorophenyl)-5-(3-chloroprop-1-yl)-1,2,4-thiadiazole (4m) Synthesized according to the general procedure from 4-chlorobenzamidine hydrochloride (191 mg) and 1-bromo-3-chloropropane (236 mg). Pale yellow liquid (224 mg, 73% yield), containing a small amount of 4-chlorobenzonitrile as an impurity. ¹H NMR (600 MHz, CDCl₃) δ 8.23-8.17 (m, 1H), 7.49-7.38 (m, 1H), 3.72 (t, J=6.1 Hz, 1H), 3.51 (t, J=6.9 Hz, 1H), 2.35 (tt, J=6.9, 6.1 Hz, 1H). ¹³C NMR (151 MHz, CDCl₃) δ 187.1, 171.4, 136.7, 131.0, 129.7, 129.1, 43.2, 31.7, 31.4. HRMS (+ESI-QTOF) m/z: [M+H]⁺ calcd for C₁₁H₁₁Cl₂N₂S₂⁺ 304.9735; found: 304.9734.

5-((2-bromo-5-methoxybenzyl)thio)-3-(4-chlorophenyl)-1,2,4-thiadiazole (4n) Synthesized according to the general procedure from 4-chlorobenzamidine hydrochloride (191 mg) and 2-bromo-5-methoxybenzyl bromide (420 mg). White platelets (311 mg, 73%). ¹H NMR (600 MHz, CDCl₃) δ 8.27-8.22 (m, 2H), 7.48 (d, J=8.8 Hz, 1H), 7.46-7.43 (m, 2H), 7.18 (d, J=3.0 Hz, 1H), 6.74 (dd, J=8.8, 3.1 Hz, 1H), 4.67 (s, 2H), 3.74 (s, 3H). ¹³C NMR (151 MHz, CDCl₃) δ 186.8, 171.0, 159.0, 136.6, 136.0, 133.8, 131.0, 129.6, 129.0, 116.8, 115.5, 115.0, 55.5, 39.0. HRMS (+ESI-QTOF) m/z: [M+H]⁺ calcd for C₁₆H₁₃BrClN₂OS₂⁺ 426.9336; found: 426.9340.

3-(4-Bromophenyl)-5-ethylthio-1,2,4-thiadiazole (4o) Synthesized according to the general procedure from 4-bromobenzamidine hydrochloride (236 mg) and bromoethane (131 mg). Light tan crystalline solid (223 mg, 74% yield). mp 36-37° C. ¹H NMR (400 MHz, CDCl₃) δ 8.18-8.10 (m, 2H), 7.63-7.55 (m, 2H), 3.33 (q, J=7.4 Hz, 2H), 1.53 (t, J=7.4 Hz, 3H). ¹³C NMR (101 MHz, CDCl₃) δ 188.1, 171.5, 132.0, 131.6, 129.9, 125.0, 28.8, 14.4. HRMS (+ESI-QTOF) m/z: [M+H]⁺ calcd for C₁₀H₁₀BrN₂S₂⁺ 300.9463; found: 300.9464.

5-Ethylthio-3-(4-(trifluoromethyl)phenyl)-1,2,4-thiadiazole (4p) Synthesized according to GP1 from 4-(trifluoromethyl)benzamidine hydrochloride dihydrate (261 mg) and bromoethane (131 mg). Eluent: 100:0 to 95:5 hexane:ethyl acetate. Colorless oil (197 mg, 68% yield). ¹H NMR (400 MHz, CDCl₃) δ 8.42-8.35 (m, 2H), 7.75-7.68 (m, 2H), 3.35 (q, J=7.4 Hz, 3H), 1.54 (t, J=7.4 Hz, 3H). ¹³C NMR (101 MHz, CDCl₃) δ 188.5, 171.1, 135.7, 132.1 (q, J=32.5 Hz), 128.7, 125.8 (q, J=3.9 Hz), 124.1 (q, J=273.3 Hz), 28.9, 14.4. ¹⁹F NMR (376 MHz, CDCl₃) δ −62.8. HRMS (+ESI-QTOF) m/z: [M+H]⁺ calcd for C₁₁H₁₀F₃N₂S₂⁺ 291.0232; found: 291.0232.

5-Ethylthio-3-(4-methoxyphenyl)-1,2,4-thiadiazole (4q) Synthesized according to the general procedure from 4-methoxybenzamidine hydrochloride (187 mg) and bromoethane (131 mg). Eluent: 100:0 to 85:15 hexane:ethyl acetate. White crystalline solid (147 mg, 58% yield). mp 54-56° C. ¹H NMR (400 MHz, CDCl₃) δ 8.25-8.17 (m, 2H), 7.01-6.93 (m, 2H), 3.87 (s, 3H), 3.32 (q, J=7.4 Hz, 2H), 1.53 (t, J=7.4 Hz, 3H). ¹³C NMR (101 MHz, CDCl₃) δ 187.3, 172.4, 161.5, 130.0, 125.7, 114.1, 55.5, 28.8, 14.4. HRMS (+ESI-QTOF) m/z: [M+H]⁺ calcd for C₁₁H₁₃N₂OS₂⁺ 253.0464; found: 253.0466.

5-Ethylthio-3-(3-methoxyphenyl)-1,2,4-thiadiazole (4r) Synthesized according to the general procedure from 3-methoxybenzamidine hydrochloride (187 mg) and bromoethane (131 mg). Extracted with ethyl acetate. Eluent: 100:0 to 80:20 hexane:ethyl acetate. Colorless oil (154 mg, 61% yield). ¹H NMR (400 MHz, CDCl₃) l 7.88 (ddd, J=7.6, 1.5, 1.0 Hz, 1H), 7.82 (dd, J=2.7, 1.5 Hz, 1H), 7.37 (dd, J=8.3, 7.6 Hz, 1H), 7.01 (ddd, J=8.3, 2.7, 1.0 Hz, 1H), 3.89 (s, 3H), 3.33 (q, J=7.4 Hz, 2H), 1.53 (t, J=7.4 Hz, 3H). ¹³C NMR (101 MHz, CDCl₃) δ 187.6, 172.4, 160.0, 133.9, 129.8, 121.0, 117.0, 113.0, 55.6, 28.8, 14.4. HRMS (+ESI-QTOF) m/z: [M+H]⁺ calcd for C₁₁H₁₃N₂OS₂⁺ 253.0464; found: 253.0466.

5-((2-Methoxyethyl)thio)-3-(3-methoxyphenyl)-1,2,4-thiadiazole (4s) Synthesized according to the general procedure from 3-methoxybenzamidine hydrochloride (187 mg) and bromoethyl methyl ether (167 mg). Extracted with ethyl acetate. Eluent: 100:0 to 80:20 hexane:ethyl acetate. Colorless oil (186 mg, 66% yield). ¹H NMR (600 MHz, CDCl₃) δ 7.86 (ddd, J=7.7, 1.5, 1.0 Hz, 2H), 7.80 (dd, J=2.7, 1.5 Hz, 1H), 7.37 (dd, J=8.3, 7.7 Hz, 1H), 7.00 (ddd, J=8.3, 2.7, 1.0 Hz, 1H), 3.88 (s, 3H), 3.79 (t, J=6.2 Hz, 2H), 3.54 (t, J=6.2 Hz, 2H), 3.42 (s, 3H). ¹³C NMR (151 MHz, CDCl₃) δ 187.2, 172.1, 159.9, 133.8, 129.8, 121.0, 116.9, 113.0, 70.5, 59.0, 55.5, 34.1. HRMS (+ESI-QTOF) m/z: [M+H]⁺ calcd for C₁₂H₁₅N₂O₃S₂⁺ 283.0569; found: 283.0569.

tert-Butyl (2-((3-methoxyphenyl)-1,2,4-thiadiazol-5-yl)thio)ethyl)carbamate (4t) Synthesized according to the general procedure from 3-methoxybenzamidine hydrochloride (187 mg) and N-Boc-2-bromoethylamine (269 mg). Extracted with ethyl acetate. Eluent: 100:0 to 80:20 hexane:ethyl acetate to give a white, fibrous solid (192 mg, 52% yield). mp 86-87° C. ¹H NMR (600 MHz, CDCl₃) δ 7.85 (ddd, J=7.6, 1.5, 1.0 Hz, 1H), 7.79 (dd, J=2.7, 1.5 Hz, 1H), 7.36 (t, J=7.9 Hz, 1H), 7.00 (ddd, J=8.3, 2.7, 1.0 Hz, 1H), 5.22 (s, 1H), 3.88 (s, 3H), 3.60 (q, J=6.2 Hz, 2H), 3.47 (t, J=6.3 Hz, 2H), 1.43 (s, 9H). ¹³C NMR (151 MHz, CDCl₃) δ 186.8, 172.2, 159.9, 155.9, 133.7, 129.8, 121.0, 116.9, 113.1, 79.8, 55.5, 40.2, 34.6, 28.5. HRMS (+ESI-QTOF) m/z: [M+H]⁺ calcd for C₁₆H₂₁N₃O₃S₂⁺ 368.1097; found: 368.1096.

5-Ethylthio-3-(3-toluyl)-1,2,4-thiadiazole (4u) Synthesized according to the general procedure from 3-methylbenzamidine hydrochloride (171 mg) and bromoethane (131 mg). Eluent: 100:0 to 95:5 hexane:ethyl acetate. Colorless oil (166 mg, 70% yield). ¹H NMR (400 MHz, CDCl₃) δ 8.09 (s, 1H), 8.07 (d, J=7.6 Hz, 1H), 7.36 (t, J=7.6 Hz, 1H), 7.27 (d, J=7.6 Hz, 1H), 3.34 (q, J=7.4 Hz, 1H), 2.43 (s, 1H), 1.54 (t, J=7.4 Hz, 2H). ¹³C NMR (101 MHz, CDCl₃) δ 187.6, 172.8, 138.5, 132.6, 131.3, 129.0, 128.7, 125.6, 28.8, 21.6, 14.4. HRMS (+ESI-QTOF) m/z: [M+H]⁺ calcd for C₁₁H₁₃N₂S₂⁺ 237.0515; found: 237.0518.

5-Ethylthio-3-(4-nitrophenyl)-1,2,4-thiadiazole (4v) Synthesized according to the general procedure from 4-nitrobenzamidine hydrochloride (202 mg) and bromoethane (131 mg). Eluent: 100:0 to 80:20 hexane:ethyl acetate. Pale yellow prisms (174 mg, 65% yield). mp 103-104° C. ¹H NMR (400 MHz, CDCl₃) δ 8.49-8.41 (m, 1H), 8.36-8.28 (m, 1H), 3.36 (q, J=7.4 Hz, 1H), 1.56 (t, J=7.4 Hz, 2H). ¹³C NMR (101 MHz, CDCl₃) δ 189.0, 170.2, 149.0, 138.0, 129.3, 124.1, 29.0, 14.4. HRMS (+ESI-QTOF) m/z: [M+H]⁺ calcd for C₁₀H₁₀N₃O₂S₂⁺ 268.0209; found: 268.0207.

5-Ethylthio-3-(pyridin-2-yl)-1,2,4-thiadiazole (4w) Synthesized according to the general procedure from pyridine-2-carboxamidine hydrochloride (158 mg) and bromoethane (131 mg). Extracted with ethyl acetate. Eluent: 100:0 to 20:80 hexane:ethyl acetate. Pale yellow oil (139 mg, 62% yield). ¹H NMR (400 MHz, CDCl₃) δ 8.78 (ddd, J=4.8, 1.8, 0.9 Hz, 1H), 8.31 (dt, J=7.9, 1.1 Hz, 1H), 7.83 (td, J=7.7, 1.8 Hz, 1H), 7.37 (ddd, J=7.6, 4.8, 1.2 Hz, 1H), 3.33 (q, J=7.4 Hz, 2H), 1.53 (t, J=7.4 Hz, 3H). ¹³C NMR (101 MHz, CDCl₃) δ 189.0, 171.8, 150.5, 150.3, 137.1, 124.8, 123.9, 28.9, 14.2. HRMS (+ESI-QTOF) m/z: [M+H]⁺ calcd for C₉H₁₀N₃S₂⁺ 224.0311; found: 224.0312.

5-Ethylthio-3-(pyridin-4-yl)-1,2,4-thiadiazole (4x) Synthesized according to the general procedure from pyridine-4-carboxamidine hydrochloride (158 mg) and bromoethane (131 mg). Extracted with ethyl acetate. Eluent: 85:15 to 0:100 hexane:ethyl acetate. White crystalline solid (117 mg, 52% yield). mp 49-50° C. ¹H NMR (400 MHz, CDCl₃) δ 8.74 (d, J=6.2 Hz, 2H), 8.09 (d, J=6.2 Hz, 2H), 3.34 (q, J=7.4 Hz, 2H), 1.53 (t, J=7.4 Hz, 4H). ¹³C NMR (101 MHz, CDCl₃) δ 188.9, 170.3, 150.7, 139.2, 122.1, 28.9, 14.3. HRMS (+ESI-QTOF) m/z: [M+H]⁺ calcd for C₉H₁₀N₃S₂⁺ 224.0311; found: 224.0314.

5-Ethylthio-3-(pyrazin-2-yl)-1,2,4-thiadiazole (4y) Synthesized according to the general procedure from pyrazine-2-carboxamidine hydrochloride (159 mg) and bromoethane (131 mg). Extracted with ethyl acetate. Eluent: 100:0 to 20:80 hexane:ethyl acetate. White crystalline solid (132 mg, 59% yield). mp 60-62° C. ¹H NMR (400 MHz, CDCl₃) δ 9.53 (d, J=1.5 Hz, 1H), 8.73 (dd, J=2.5, 1.5 Hz, 1H), 8.66 (d, J=2.5 Hz, 1H), 3.36 (q, J=7.4 Hz, 2H), 1.54 (t, J=7.4 Hz, 3H). ¹³C NMR (101 MHz, CDCl₃) δ 189.8, 169.5, 146.1, 145.6, 145.4, 144.7, 29.0, 14.2. HRMS (+ESI-QTOF) m/z: [M+H]⁺ calcd for C₈H₉N₄S₂⁺ 225.0263; found: 225.0263.

5-Ethylthio-3-methyl-1,2,4-thiadiazole (5a) Synthesized according to the general procedure from acetamidine hydrochloride (94.5 mg) and bromoethane (131 mg). Eluent: 100:0 to 95:5 hexane:ethyl acetate. Colorless oil with unpleasant odor (89.2 mg, 56% yield). ¹H NMR (400 MHz, CDCl₃) δ 3.23 (q, J=7.4 Hz, 2H), 2.61 (s, 3H), 1.48 (t, J=7.4 Hz, 3H). ¹³C NMR (101 MHz, CDCl₃) δ 187.5, 173.1, 28.7, 19.0, 14.3. HRMS (+ESI-QTOF) m/z: [M+H]⁺ calcd for C₅H₉N₂S₂⁺ 161.0202; found: 161.0200.

3-Cyclopropyl-5-ethylthio-1,2,4-thiadiazole (5b) Synthesized according to the general procedure from cyclopropanecarboxamidine hydrochloride (121 mg) and bromoethane (131 mg). Eluent: 100:0 to 95:5 hexane:ethyl acetate. Colorless oil. (121 mg, 65% yield). ¹H NMR (400 MHz, CDCl₃) δ 3.20 (q, J=7.4 Hz, 2H), 2.26 (tt, J=8.2, 4.9 Hz, 1H), 1.46 (t, J=7.4 Hz, 3H), 1.14-1.09 (m, 2H), 1.05-1.00 (m, 2H). ¹³C NMR (101 MHz, CDCl₃) δ 187.0, 178.2, 28.6, 14.3, 13.8, 9.5. HRMS (+ESI-QTOF) m/z: [M+H]⁺ calcd for C₇H₁₁N₂S₂⁺ 187.0358; found: 187.0356.

3-(2,6-Dichlorobenzyl)-5-ethylthio-1,2,4-thiadiazole (5c) Synthesized according to the general procedure from 2-(2,6-dichlorophenyl)ethanimidamide hydrochloride (240 mg) and bromoethane (131 mg). Eluent: 100:0 to 95:5 hexane:ethyl acetate to yield a pale yellow solid (172 mg, 56%). ¹H NMR (400 MHz, CDCl₃) δ 7.35 (d, J=8.0 Hz, 1H), 7.18 (t, J=8.0 Hz, 1H), 4.60 (s, 1H), 3.22 (q, J=7.4 Hz, 1H), 1.46 (t, J=7.4 Hz, 1H). ¹³C NMR (101 MHz, CDCl₃) δ 187.82, 172.40, 136.32, 133.67, 128.87, 128.28, 35.07, 28.76, 14.26. HRMS (+ESI-QTOF) m/z: [M+H]⁺ calcd for C₁₁H₁₁Cl₂N₂S₂⁺ 304.9735; found: 304.9739.

Ethyl 2-(5-ethylthio-1,2,4-thiadiazol-3(2H)-ylidene)acetate (5d) Synthesized according to the general procedure from 2-carbethoxyacetamidine hydrochloride (167 mg) and bromoethane (131 mg). Eluent: 100:0 to 85:15 hexane:ethyl acetate. White crystalline solid (165 mg, 71% yield). mp 128-129° C. ¹H NMR (400 MHz, CDCl₃) δ 5.76 (broad s, 2H), 4.36 (q, J=7.1 Hz, 2H), 2.99 (q, J=7.4 Hz, 2H), 1.46 (t, J=7.4 Hz, 3H), 1.40 (t, J=7.1 Hz, 3H). ¹³C NMR (101 MHz, CDCl₃) δ 173.9, 165.2, 163.2, 109.4, 61.1, 28.1, 14.5, 13.5. HRMS (+ESI-QTOF) m/z: [M+H]⁺ calcd for C₈H₁₃N₂O₂S₂⁺233.0413; found: 233.0416.

2-(5-Ethylthio-1,2,4-thiadiazol-3(2H)-ylidene)acetamide (5e) Synthesized according to the general procedure from malonamamidine hydrochloride (138 mg) and bromoethane (131 mg). Eluent: 100:0 to 20:80 hexane:ethyl acetate. White crystalline solid (117 mg, 57% yield). mp 140-142° C. ¹H NMR (400 MHz, CDCl₃) δ 6.5 (broad s, 2H), 6.10 (broad s, 2H), 3.07 (q, J=7.4 Hz, 2H), 1.43 (t, J=7.4 Hz, 3H). ¹³C NMR (101 MHz, CDCl₃) δ 166.3, 164.8, 162.4, 114.8, 31.6, 14.4. HRMS (+ESI-QTOF) m/z: [M+H]⁺ calcd for C₆H₁₀N₃OS₂⁺ 204.0260; found: 204.0262.

5-Ethylthio-3-methoxy-1,2,4-thiadiazole (5f) Synthesized according to the general procedure from O-methylisourea hemisulfate (121 mg) and bromoethane (131 mg). Solvent: 9:1 acetonitrile:DMPU. Eluent: 100:0 to 95:5 hexane:ethyl acetate. White crystals (83 mg, 47% yield). mp 35-37° C. ¹H NMR (400 MHz, CDCl₃) δ 7.26, 4.06, 3.26, 3.24, 3.22, 3.20, 1.48, 1.46, 1.45. ¹³C NMR (101 MHz, CDCl₃) δ 188.8, 171.2, 57.3, 28.4, 14.4. HRMS (+ESI-QTOF) m/z: [M+H]⁺ calcd for C₅H₉N₂OS₂⁺ 177.0151; found: 177.0148.

3,5-Bis(ethylthio)-1,2,4-thiadiazole (5h) Synthesized according to the general procedure from S-ethylisothiourea hydrobromide (185 mg) and bromoethane (131 mg). Eluent: 100:0 to 85:15 hexane:ethyl acetate to yield a pale yellow oil (116 mg, 56% yield). ¹H NMR (400 MHz, CDCl₃) δ 3.25 (q, J=7.4 Hz, 2H), 3.2 (q, J=7.4 Hz, 2H), 1.47 (t, J=7.4 Hz, 3H), 1.43 (t, J=7.4 Hz, 3H). ¹³C NMR (101 MHz, CDCl₃) δ 187.9, 171.1, 28.7, 26.6, 14.8, 14.3. HRMS (+ESI-QTOF) m/z: [M+H]⁺ calcd for C₆H₁₀N₂S₃⁺ 206.0006; found: 206.0003.

5-Ethylthio-3-dimethylamino-1,2,4-thiadiazole (5i) Synthesized according to the general procedure from N,N-dimethylguanidine hemisulfate (136 mg) and bromoethane (131 mg). Solvent: 9:1 acetonitrile:DMPU. Eluent: 100:0 to 70:30 hexane:ethyl acetate. White solid (85 mg, 45% yield). mp 116-118° C. ¹H NMR (400 MHz, CDCl₃) δ 3.165 (q, J=7.4 Hz, 2H), 3.162 (s, 6H), 1.47 (t, J=7.4 Hz, 3H). ¹³C NMR (101 MHz, CDCl₃) δ 186.1, 171.3, 39.1, 28.3, 14.5. HRMS (+ESI-QTOF) m/z: [M+H]⁺ calcd for C₆H₁₂N₃S₂⁺ 190.0467; found: 190.0468.

3-(4-Benzylpiperazin-1-yl)-5-ethylthio-1,2,4-thiadiazole (5j) Synthesized according to the general procedure from 4-benzylpiperazine-1-carboximidamide hydroiodide (346 mg) and bromoethane (131 mg). Eluent: 100:0 to 80:20 hexane:ethyl acetate. Colorless liquid (188 mg, 59%). ¹H NMR (600 MHz, CDCl₃) δ 7.37-7.30 (m, 4H), 7.30-7.24 (m, 1H), 3.72-3.67 (m, 4H), 3.55 (s, 2H), 3.16 (q, J=7.4 Hz, 2H), 2.55-2.50 (m, 4H), 1.46 (t, J=7.4 Hz, 3H). ¹³C NMR (151 MHz, CDCl₃) δ 186.5, 170.7, 138.1, 129.3, 128.4, 127.3, 63.3, 52.8, 46.8, 28.3, 14.4. HRMS (+ESI-QTOF) m/z: [M+H]⁺ calcd for C₁₅H₂₁N₄S₂⁺ 321.1202; found: 321.1199.

N^{α}-benzoyl-O-ethyl-N₆-(5-ethylthio-1,2,4-thiadiazol-3-yl)ornithine (5k) Synthesized according to the general procedure from N^{α}-benzoyl−1-arginine ethyl ester hydrochloride (343 mg) and bromoethane (131 mg). Extracted with ethyl acetate. Eluent: 100:0 to 40:60 hexane:ethyl acetate. White botryoidal crystals (216 mg, 53% yield). mp 92-94° C. ¹H NMR (400 MHz, CDCl₃) δ 7.84-7.78 (m, 2H), 7.55-7.47 (m, 1H), 7.47-7.41 (m, 2H), 6.86 (d, J=7.7 Hz, 1H), 5.19 (t, J=6.0 Hz, 1H), 4.85 (td, J=7.4, 5.2 Hz, 1H), 4.23 (q, J=7.1 Hz, 2H), 3.51-3.39 (m, 2H), 3.14 (q, J=7.4 Hz, 2H), 2.07 (ddt, J=13.4, 9.6, 5.6 Hz, 1H), 1.89 (dddd, J=13.5, 8.8, 7.2, 6.1 Hz, 1H), 1.84-1.67 (m, 2H), 1.44 (t, J=7.4 Hz, 3H), 1.29 (t, J=7.1 Hz, 3H). ¹³C NMR (101 MHz, CDCl₃) δ 186.6, 172.6, 169.3, 167.3, 134.1, 131.9, 128.7, 127.2, 61.9, 52.5, 43.1, 30.1, 28.3, 25.8, 14.4, 14.3. HRMS (+ESI-QTOF) m/z: [M+H]⁺ calcd for C₁₈H₂₄N₄O₃S₂⁺ 409.1363; found: 409.1365.

2-(4-chlorophenyl)-4-carbmethoxy-5-((carbmethoxymethyl)thio)imidazole (6) 4-Chlorobenzamidine hydrochloride (2 mmol) and DBU were dissolved in acetonitrile (10 mL), and carbon disulfide (2.2 mmol, 1.1 eq) was added in one portion. The dark orange solution was stirred at room temperature for thirty minutes, and methyl bromoacetate (4.4 mmol, 2.2 eq) was added in one portion, and the solution turned bright yellow, then deep red. The reaction was stirred for 12 hours; then NCS (1.2 eq) was added in one portion, followed by stirring for 30 minutes. The reaction was quenched by the addition of 1M sodium thiosulfate (5 mL) and stirring for 30 minutes, and the mixture was diluted with water (50 mL) and the product was extracted with ethyl acetate (30 mL). The organic layer was washed with water, then 1 M sodium hydroxide, then 1 M hydrochloric acid, and then brine (50 mL each), and then dried over anhydrous magnesium sulfate. The solvent was removed under vacuum, and the residue was subjected to flash chromatography using 100:0 to 75:25 hexane:ethyl acetate. White, fibrous crystals. ¹H NMR (400 MHz, CDCl₃) δ 7.78-7.69 (m, 2H), 7.42-7.36 (m, 2H), 4.08 (s, 2H), 3.80 (s, 2H), 3.76 (s, 3H). ¹³C NMR (101 MHz, CDCl₃) δ 168.6, 167.4, 161.4, 158.5, 135.6, 131.9, 131.5, 128.1, 122.0, 53.1, 52.6, 35.2. HRMS (+ESI-QTOF) m/z: [M+H]⁺ calcd for C₁₄H₁₄N₂O₄S⁺ 341.0357; found: 341.0355.

5-(2-(Carbmethoxy)ethylthio)-3-(4-chlorophenyl)-1,2,4-thiadiazole (9a) Synthesized according to the general procedure from 4-chlorobenzamidine hydrochloride (191 mg) and methyl acrylate (125 μL, 1.5 eq) instead of an alkyl halide. Extracted with ethyl acetate. Eluent: 100:0 to 85:15 hexane:ethyl acetate; additional recrystallization from hot hexane/ethyl acetate yielded white, fibrous crystals (192 mg, 61% yield). mp 89-90° C. ¹H NMR (400 MHz, CDCl₃) δ 8.22-8.14 (m, 2H), 7.46-7.38 (m, 2H), 3.73 (s, 3H), 3.60 (t, J=7.0 Hz, 2H), 2.94 (t, J=7.0 Hz, 2H). ¹³C NMR (101 MHz, CDCl₃) δ 188.1, 171.0, 136.8, 130.8, 129.6, 129.2, 72.4, 41.4, 29.6, 10.1. HRMS (+ESI-QTOF) m/z: [M+H]⁺ calcd for C₁₂H₁₂ClN₂O₂S₂⁺ 315.0023; found: 315.0023.

5-((3-Oxobut-1-yl)thio)-3-(4-chlorophenyl)-1,2,4-thiadiazole (9b) Synthesized according to the general procedure from 4-chlorobenzamidine hydrochloride (191 mg) and methyl vinyl ketone (135 μL, 1.5 eq) instead of an alkyl halide. Extracted with ethyl acetate. Eluent: 100:0 to 85:15 hexane:ethyl acetate. Pale yellow oil (32 mg, 11% yield). ¹H NMR (600 MHz, CDCl₃) δ 8.22-8.17 (m, 1H), 7.46-7.40 (m, 1H), 3.54 (t, J=6.7 Hz, 1H), 3.07 (t, J=6.7 Hz, 1H), 2.21 (s, 1H). ¹³C NMR (151 MHz, CDCl₃) δ 187.5, 171.3, 136.7, 129.7, 129.1, 42.9, 30.2, 27.9. HRMS (+ESI-QTOF) m/z: [M+H]⁺ calcd for C₁₂H₁₂ClN₂OS₂⁺ 299.0074; found: 299.0071.

5-(2-Hydroxy-1-butylthio)-3-(4-chlorophenyl)-1,2,4-thiadiazole (9c) Synthesized according to the general procedure from 4-chlorobenzamidine hydrochloride (191 mg) and 1,2-epoxybutane (130 μL, 1.5 eq) instead of an alkyl halide. Extracted with ethyl acetate. Eluent: 100:0 to 80:20 hexane:ethyl acetate to yield a colorless oil which solidified upon standing overnight into white, fibrous crystals (143 mg, 48% yield). mp 77-79° C. ¹H NMR (400 MHz, CDCl₃) δ 8.19-8.11 (m, 2H), 7.48-7.39 (m, 2H), 4.05-3.93 (m, 1H), 3.58 (dd, J=14.0, 3.2 Hz, 1H), 3.33 (dd, J=14.0, 7.5 Hz, 1H), 3.20 (d, J=4.3 Hz, 1H), 1.69 (qd, J=7.4, 6.3 Hz, 2H), 1.05 (t, J=7.4 Hz, 3H). ¹³C NMR (101 MHz, CDCl₃) δ 188.0, 170.8, 136.7, 130.6, 129.5, 129.0, 72.2, 41.3, 29.5, 10.0. HRMS (+ESI-QTOF) m/z: [M+H]⁺ calcd for C₁₂H₁₅ClN₂OS₂⁺ 301.0231; found: 301.0235.

### General Procedure for the Synthesis of 5-methoxy-1,2,4-thiadiazoles (GP2)

In a modification of GP1, amidine hydrochloride (1.0 mmol) and DBU (312 mg, 2.05 mmol) were dissolved in methanol (10 mL), followed by the addition of carbon disulfide (114 mg, 1.5 mmol). The pale yellow solution was stirred for 2 hours at 22° C., and then ethyl bromide (164 mg, 1.5 mmol) was added in one portion at 22° C. and the reaction was stirred for 18 hours. The yellow solution was then cooled to 0° C., and NCS (147 mg, 1.1 eq). The reaction was stirred at 22° C. for 30 minutes, and excess NCS was quenched by the addition of 1 M sodium thiosulfate solution (2 mL). The reaction was worked up as in GP1. Thiadiazoles 10 were purified by flash chromatography using 100:0 to 80:20 hexane:ethyl acetate as eluent.

3-phenyl-5-methoxy-1,2,4-thiadiazole (10a) Synthesized according to GP2 from benzamidine hydrochloride (157 mg). Eluent: 100:0 to 75:25 hexane:ethyl acetate. Pale yellow liquid (49.7 mg, 26%). ¹H NMR (400 MHz, CDCl₃) δ 8.24-8.13 (m, 2H), 7.50-7.40 (m, 3H), 4.26 (s, 3H). ¹³C NMR (101 MHz, CDCl₃) δ 191.7, 168.5, 133.1, 130.4, 128.7, 128.0, 60.3. HRMS (+ESI-QTOF) m/z: [M+H]⁺ calcd for C₉H₉N₂OS⁺ 193.0430; found: 193.0426.

3-(4-fluorophenyl)-5-methoxy-1,2,4-thiadiazole (10b) Synthesized according to GP2 from 4-fluorobenzamidine hydrochloride (175 mg). Eluent: 100:0 to 75:25 hexane:ethyl acetate. Remaining 11b was removed by adding hexane (1 mL) and cooling to 0° C., followed by filtering through a glass pipet with a cotton plug; the hexane was then removed from the filtrate under vacuum to yield 10b as a white solid (62.9 mg, 30%), containing approximately 5% 11b). mp 73-75° C. ¹H NMR (400 MHz, CDCl₃) δ 8.24-8.15 (m, 2H), 7.16-7.08 (m, 3H), 4.24 (s, 3H). ¹³C NMR (101 MHz, CDCl₃) δ 191.7, 167.4, 164.2 (d, J=250.2 Hz), 130.0 (d, J=8.7 Hz), 129.5 (d, J=3.1 Hz), 115.7 (d, J=21.9 Hz), 60.4. ¹⁹F NMR (376 MHz, CDCl₃) δ −110.35 (tt, J=8.9, 5.3 Hz). HRMS (+ESI-QTOF) m/z: [M+H]⁺ calcd for C₉H₈FN₂OS⁺ 211.0336; found: 211.0333.

3-(4-chlorophenyl)-5-methoxy-1,2,4-thiadiazole (10c) Synthesized according to GP2 from 4-chlorobenzamidine hydrochloride (192 mg). Eluent: 100:0 to 75:25 hexane:ethyl acetate. Remaining 11c was removed as in 10b to yield 10c as a white crystalline solid (95.1 mg, 42%). mp 137-139° C. ¹H NMR (600 MHz, CDCl₃) δ 8.16-8.11 (m, 2H), 7.44-7.38 (m, 2H), 4.24 (s, 3H). ¹³C NMR (151 MHz, CDCl₃) δ 191.8, 167.4, 136.4, 131.6, 129.3, 128.9, 60.4. HRMS (+ESI-QTOF) m/z: [M+H]⁺ calcd for C₉H₈ClN₂OS⁺ 227.0040; found: 227.0038.

5-methoxy-3-(3-methoxyphenyl)-1,2,4-thiadiazole (10d) Synthesized according to GP2 from 3-methoxybenzamidine hydrochloride (187 mg). Eluent: 100:0 to 80:20 hexane:ethyl acetate. Remaining 11d was removed as in 10b. White crystalline solid (108 mg, 49%). mp 73-75° C. ¹H NMR (600 MHz, CDCl₃) δ 7.81 (dt, J=7.6, 1.3 Hz, 1H), 7.75 (dd, J=2.7, 1.5 Hz, 1H), 7.35 (t, J=8.0 Hz, 1H), 6.99 (ddd, J=8.2, 2.7, 1.0 Hz, 1H), 4.24 (s, 3H), 3.88 (s, 3H). ¹³C NMR (151 MHz, CDCl₃) δ 191.5, 168.2, 159.8, 134.3, 129.6, 120.5, 116.7, 112.5, 60.2, 55.4. HRMS (+ESI-QTOF) m/z: [M+H]⁺ calcd for C₁₀H₁₁N₂O₂S⁺ 223.0536; found: 223.0536.

5-methoxy-3-(4-nitrophenyl)-1,2,4-thiadiazole (10e) Synthesized according to GP2 from 4-nitrobenzamidine hydrochloride (202 mg). Eluent: 100:0 to 75:25 hexane:ethyl acetate. Pale yellow crystalline solid (151 mg, 64%). mp 138-140° C. ¹H NMR (600 MHz, CDCl₃) δ 8.40-8.34 (m, 1H), 8.31-8.26 (m, 1H), 4.28 (s, 1H). ¹³C NMR (151 MHz, CDCl₃) δ 192.2, 166.1, 148.8, 138.4, 128.8, 124.0, 60.6. HRMS (+ESI-QTOF) m/z: [M+H]⁺ calcd for C₉H₈N₂O₃S⁺ 238.0281; found: 238.0282.

### General Procedure for the Synthesis of 2-methoxy-4,6-disubstituted-1,3,5-triazines (GP3)

In a modification of GP1, amidine hydrochloride (2.0 mmol) and DBU (624 mg, 4.1 mmol) were dissolved in methanol (15 mL), followed by the addition of carbon disulfide (76 mg, 1 mmol) and ethyl bromide (273 mg, 2.5 mmol) in one portion at 22° C. The reaction was stirred at room temperature for 2 hours, and then heated at reflux 6 hours. The excess methanol was removed from the pale yellow solution under vacuum, and the reaction was worked up as in GP1. Triazines were purified by flash chromatography using 100:0 to 80:20 hexane:ethyl acetate as eluent.

2-methoxy-4,6-diphenyl-1,3,5-triazine (11a) Synthesized according to GP3 from benzamidine hydrochloride (313 mg). White crystalline solid (87.0 mg, 33%). mp 109-111° C. ¹H NMR (400 MHz, CDCl₃) δ 8.69-8.62 (m, 4H), 7.62-7.57 (m, 2H), 7.56-7.51 (m, 4H), 4.23 (s, 3H). ¹³C NMR (101 MHz, CDCl₃) δ 173.7, 172.0, 135.8, 132.8, 129.2, 128.7, 55.1. HRMS (+ESI-QTOF) m/z: [M+H]⁺ calcd for C₁₆H₁₄N₃O⁺ 264.1131; found: 264.1129.

4,6-bis(4-fluorophenyl)-2-methoxy-1,3,5-triazine (11b) Synthesized according to GP3 from 4-fluorobenzamidine hydrochloride (349 mg). White crystalline solid (136 mg, 45%). mp 173-174° C. ¹H NMR (600 MHz, CDCl₃) δ 8.66-8.59 (m, 4H), 7.23-7.16 (m, 4H), 4.20 (s, 3H). ¹³C NMR (151 MHz, CDCl₃) δ 172.6, 171.8, 165.9 (d, J=253.4 Hz), 131.7 (d, J=2.9 Hz), 131.4 (d, J=8.9 Hz), 115.7 (d, J=21.8 Hz), 55.0. ¹⁹F{¹H} NMR (565 MHz, CDCl₃) δ −106.6. HRMS (+ESI-QTOF) m/z: [M+H]⁺ calcd for C₁₆H₁₂F₂N₃O⁺ 300.0943; found: 300.0941.

4,6-bis(4-chlorophenyl)-2-methoxy-1,3,5-triazine (11c) Synthesized according to GP3 from 4-chlorobenzamidine hydrochloride (382 mg). White fibrous solid (130 mg, 39%). mp 192-194° C. ¹H NMR (600 MHz, CDCl₃) δ 8.58-8.53 (m, 4H), 7.52-7.47 (m, 4H), 4.21 (s, 3H). ¹³C NMR (151 MHz, CDCl₃) δ 172.9, 172.0, 139.3, 134.1, 130.5, 129.1, 55.3. HRMS (+ESI-QTOF) m/z: [M+H]⁺ calcd for C₁₆H₁₂Cl₂N₃O⁺ 332.0352; found: 332.0353.

4,6-bis(3-methoxyphenyl)-2-methoxy-1,3,5-triazine (11d) Synthesized according to GP3 from 3-methoxybenzamidine hydrochloride (373 mg). White fibrous solid (109 mg, 34%). mp 126-128° C. ¹H NMR (600 MHz, CDCl₃) δ 8.25 (dt, J=7.7, 1.3 Hz, 2H), 8.17 (dd, J=2.7, 1.5 Hz, 2H), 7.44 (t, J=7.9 Hz, 2H), 7.14 (ddd, J=8.2, 2.7, 1.0 Hz, 2H), 4.22 (s, 3H), 3.93 (s, 6H). ¹³C NMR (151 MHz, CDCl₃) δ 173.4, 171.8, 159.9, 137.1, 129.6, 121.6, 118.9, 113.7, 55.5, 55.1. HRMS (+ESI-QTOF) m/z: [M+H]⁺ calcd for C₁₈H₁₈N₃O₃⁺ 324.1343; found: 324.1342.

4,6-bis(4-chlorophenyl)-2-ethylthio-1,3,5-triazine (14) In acetonitrile (10 mL) was dissolved DBU (312 mg, 2.05 mmol) and 4-chlorobenzamidine hydrochloride (192 mg). Carbon disulfide (38 mg, 0.5 mmol) and ethyl bromide (273 mg, 2.5 eq) were added together, and the reaction was stirred at 22° C. for two hours, and then at reflux for six hours. The yellow solution was poured into water (50 mL) and extracted with ethyl acetate (3×10 mL). The combined organic layers were washed with water, then 1 M sodium hydroxide, then brine (10 mL each). The solution as dried over anhydrous magnesium sulfate, and the solvent was removed under vacuum. The crude product was purified by column chromatography using and eluent gradient of 100:0 to 75:25 hexane:ethyl acetate to yield a quantity of 3ca (38.9 mg, 30% based on CS₂) as bright yellow-orange needles, and the title compound as a white fibrous solid (33.4 mg, 18%). mp 173-174° C. ¹H NMR (400 MHz, CDCl₃) δ 8.59-8.49 (m, 4H), 7.57-7.45 (m, 4H), 3.31 (q, J=7.3 Hz, 2H), 1.51 (t, J=7.4 Hz, 3H). ¹³C NMR (101 MHz, CDCl₃) δ 183.3, 169.4, 139.2, 134.2, 130.4, 129.1, 25.2, 14.5. HRMS (+ESI-QTOF) m/z: [M+H]⁺ calcd for C₁₇H₁₄Cl₂N₃S⁺ 362.0280; found: 362.0279.

3-phenyl-5-ethoxy-1,2,4-thiadiazole (15) Synthesized according to GP2 from benzamidine hydrochloride (157 mg), with the modification of using 1:1 ethanol:acetonitrile instead of methanol as the solvent. Eluent: 100:0 to 80:20 hexane:ethyl acetate. Pale yellow liquid. ¹H NMR (400 MHz, CDCl₃) δ 8.24-8.17 (m, 2H), 7.51-7.39 (m, 3H), 4.61 (q, J=7.1 Hz, 2H), 1.52 (t, J=7.1 Hz, 3H). ¹³C NMR (101 MHz, CDCl₃) δ 191.0, 168.5, 133.2, 130.3, 128.6, 127.9, 70.1, 14.6. HRMS (+ESI-QTOF) m/z: [M+H]⁺ calcd for C₁₀H₁₁N₂OS⁺ 207.0587; found: 207.0588.

### Example 2—One-Pot Synthesis of Ketene Dithioacetals from Aldehydes and Disulfides Via an In Situ Horner-Wadsworth-Emmons Reaction

Results and Discussion

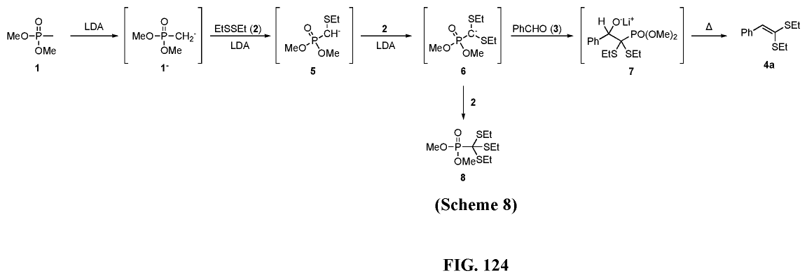

Because sulfur stabilizes carbanions (Bernasconi & Kittredge, “Carbanion Stabilization by Adjacent Sulfur: Polarizability, Resonance, or Negative Hyperconjugation? Experimental Distinction Based on Intrinsic Rate Constants of Proton Transfer from (Phenylthio)nitromethane and 1-Nitro-2-phenylethane,” J. Org. Chem. 63:1944-1953 (1998); Corey & Seebach, “Carbanions of 1,3-Dithianes. Reagents for C—C Bond Formation by Nucleophilic Displacement and Carbonyl Addition,” Angew. Chem. Int. Ed. Engl. 4:1075-1077 (1965), which are hereby incorporated by reference in their entirety), the approach to the synthesis of KDTAs began by attempting to form the anion of dimethyl bis(ethylthio)methylphosphonate (6) using 1, diethyl disulfide (2), and an excess of lithium diisopropylamide (FIG. 124), under the premise that sulfenylation occurs stepwise, and that the rate of addition of diethyl disulfide is fastest for the phosphonate anion 1⁻, followed by the monosulfenylated phosphonate 5, and the bis(sulfenyl)ated phosphonate anion 6 adds most slowly to diethyl disulfide. This pattern follows that expected from basicity and sterics. The addition of benzaldehyde (3) to a solution of the anion 6 then forms the phosphonoalkoxide 7, the expected HWE intermediate, which readily eliminates dimethylphosphate to give the expected KDTA.

Optimization of the reaction conditions is shown in Table 3. Changing the solvent from THE to diethyl ether (Table 3, Entry 2) significantly decreased the yield, likely a result of the observed decrease of the solubility of anion 1⁻ in diethyl ether. Replacing LDA with lithium 2,2,6,6-tetramethylpiperide (Table 3, Entry 3) resulted in a slight reduction of yield, while using butyllithium (Table 3, Entry 4) as the base produced only a trace of 4a, and a large quantity of ethyl butyl sulfide was detected by GCMS, as expected from the direct addition of butyllithium to diethyl disulfide. The yield was dramatically reduced if the diethyl disulfide was added rapidly to the reaction (Table 3, Entry 5), or if an excess of 2 was used (Table 3, Entry 6). Conceivably, using an excess of 2 may result in the formation of trithioorthoester 8 via the additional sulfenylation of 6 (FIG. 124). However, no effort was made to synthesize or isolate 8 from the reaction. Additionally, using excess 2a or attempting the formation of the anion 6 was attempted at 0° C. (Table 3, Entry 7) resulted in the production of substantial quantities of N-(ethylthio)diisopropylamine, as detected in the reaction mixture by GCMS. No benefit was seen when the reaction was cooled to −78° C. before the addition of the aldehyde (Table 3, Entry 8). While the elimination of dimethylphosphate to form 4a occurs readily, warming briefly to 55° C. is beneficial for completing the elimination, as omitting this warming step lowered the reaction yield (Table 3, Entry 9).

TABLE 3

| Optimization of the reaction conditions. Standard conditions: 1 | | |
|---|---|---|
| (1:1 mmol), LDA (3.3 mmol), diethyl disulfide (2, 2.2 mmol) | | |
| added dropwise, in THF (15 mL) at 0° C., then | | |
| benzaldehyde (3, 1 mmol), warming to 55° C. | | |
| 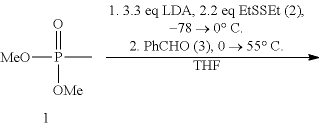 | | |
| 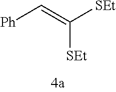 | | |
| Entry | Change from standard conditions | Yield |
| 1 | No change | 82% |
| 2 | Et₂O instead of THF | 43% |
| 3 | LiTMP instead of LDA | 76% |
| 4 | nBuLi instead of LDA | trace |
| 5 | Rapid addition of EtSSEt | 18% |
| 6 | 3 equivalents of 2 | 46% |
| 7 | Step 1 at 0° C. | 31% |
| 8 | Step 2 at −78° C. | 80% |
| 9 | Warming to 22° C. instead of 55° C. in step 2 | 67% |

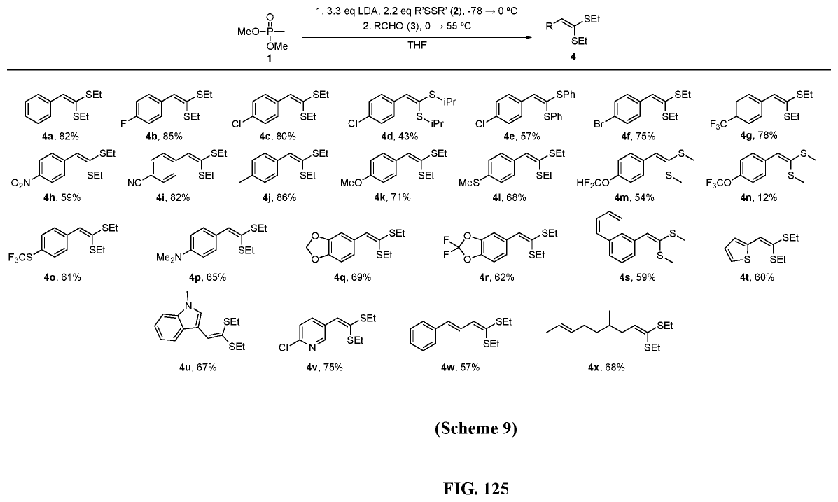

FIG. 125 shows the scope of the one-pot reaction for synthesizing a variety of ketene dithioacetals. In general, KDTAs containing electron-withdrawing groups (e.g., 4b-g,i) were produced in superior yields to those with electron-donating groups (e.g., 4k and 4l). The lower yield of the nitro derivative 4h may be a result of a competing side reactions occurring at the nitro group (Kobrich & Buck, “Nachweis und Darstellung metallierter Nitroaromaten,” Chem. Ber. 103:1412-1419 (1970), which is hereby incorporated by reference in its entirety). Both 7r-excessive and 7r-deficient heterocyclic carboxaldehydes produced their respective products (4t-v) in acceptable yields, as did both α,β-unsaturated aldehyde cinnamaldehyde (4w) and alkyl aldehyde citronellal (4x).

The use of several disulfides was also explored. While the optimization of conditions was performed using diethyl disulfide, dimethyl disulfide also gave acceptable yields of the expected products (4m and 4s). When using diisopropyl disulfide to produce 4d, the expected product was obtained; however, the yield was substantially lower than that achieved when synthesizing the ethyl analog 4c, likely due to steric hinderance of the phosphonate anion. Diphenyl disulfide (2d) also gave acceptable yields of 4e. It is unclear why the yield of 4n was so poor, especially when compared to 4m or 4o. Despite several attempts using freshly-distilled 4-trifluoromethoxybenzaldehyde, an acceptable yield of this product could not be achieved.

The use of S-methyl methanethiosulfonate (MeSSO₂Me) and dimethyl disulfide as sulfenylating reagents was explored, as thiosulfonates are generally considered more reactive and effective than disulfides for sulfenylation (Scholz, D., “Neue Synthesemethoden; 8. α-Methylthiolierung cyclischer Ketone,” Synthesis 1983:944-945 (1983); Wladislaw et al., “α-(Methylthio)benzyl Sulfones as Synthetic Intermediates. Part IV. Some New o-, m- and p-substituted α-(Methylthio)- and α,α′-Bis-(methylthio)-benzyl Sulfones,” Phosphorus, Sulfur, Silicon Relat. Elem. 48:163-167 (1990), which are hereby incorporated by reference in their entirety). However, under the normal reaction conditions, a heavy white precipitate, likely lithium methanesulfinate, formed, which interfered with stirring and resulted in almost no product. A trace of 4s was detected by GCMS when attempting to synthesize this compound using MeSSO₂Me in place of dimethyl disulfide, compared to the respectable yield of 59%.

The purified KDTAs typically have little odor and appear to be stable for at least several months when stored under argon in the dark at room temperature. However, the isolated compounds were stored at −20° C. If these compounds are left on the benchtop exposed to air and light for several days, they slowly yellow and become malodorous, and the NMRs of these samples show minor degradation.

Conclusions

Although ketenedithioacetals have been readily synthesized from EWG-stabilized carbanions in the presence of alkyl halides and carbon disulfide, the method described here permits the one-pot synthesis of KDTAs from aldehydes via a Horner-Wadsworth-Emmons reaction using readily-available dimethyl methylphosphonate and disulfides. Benzaldehydes containing electron-withdrawing substituents tended to result in somewhat better yields. However, the yield of the KDTA products were also acceptable for electron-rich aldehydes. Additionally, heterocyclic, allylic, and alkyl aldehydes were all smoothly converted to KDTAs. This simple strategy for the synthesis of ketenedithioacetals should improve the accessibility of this interesting class of molecules for use in organic synthesis.

Experimental

General Information

Anhydrous tetrahydrofuran was purchased from Acros Organics and was stored under argon. Diisopropylamine was purchased from Sigma-Aldrich and distilled under argon before use. n-Butyllithium was purchased from Sigma-Aldrich and titrated before use. Dimethyl methylphosphonate was purchased from Strem Chemicals and was distilled under vacuum before use. All disulfides were purchased from TCI and were used as received, and aldehydes were purchased from multiple sources, including Sigma-Aldrich, Acros Organics, Alfa Aesar, Matrix Scientific, Oakwood Chemicals, Combi-Blocks, Synthonix, and Chem-Impex. Liquid aldehydes were purified by distillation or column chromatography before use to remove any carboxylic acid. All reactions were performed under an argon atmosphere. Ketenedithioacetals were purified on a Buchi Pure C-810 Flash chromatography system using HPLC grade solvents on 12 g or 25 g FlashPure silica cartridges. The characterization of all compounds was performed at the Iowa State University Chemical Instrumentation Facility. NMR spectra were obtained using Avance III 600 MHz and Avance NEO 400 MHz spectrometers. Chemical shifts are reported in ppm relative to the residual solvent peak (CDCl₃: 7.26 ppm for ¹H and 77.16 ppm for ¹³C; DMSO-d₆: 2.50 for ¹H and 39.52 ppm for ¹³C; CD₃OD: 3.31 ppm for ¹H and 49.00 ppm for ¹³C) (Fulmer et al., “NMR Chemical Shifts of Trace Impurities: Common Laboratory Solvents, Organics, and Gases in Deuterated Solvents Relevant to the Organometallic Chemist,” Organometallics 29:2176-217 (2010), which are hereby incorporated by reference in their entirety) or, for ¹⁹F, an external CFCl₃ reference (0 ppm). Coupling constants are reported in Hz. HRMS analysis was performed using electrospray ionization positive ion mode (ESI+) on an Agilent QTOF 6540 mass spectrometer. Accurate mass measurement was achieved by constantly infusing a calibrant (masses: 121.0508 and 922.0098). Melting points were obtained on Stuart SMP30 melting point apparatus using a temperature ramp rate of 3° C. min⁻¹.

All reactions were performed in a fumehood, as dialkyl disulfides are foul-smelling compounds. To minimize the risk of the production of free thiol, the aqueous workup was performed in the order described in the general procedure, with basic washings taking place before acidic washings; the basic and acidic washings were not mixed. Thiolates in the basic washing may be destroyed by the careful addition of sodium hypochlorite.

General Procedure for the Synthesis of Ketenedithioacetals

A 50 mL 14/20 one-neck round bottom flask with rubber septum and stir bar was flushed with argon, and charged with diisopropylamine (344 mg, 3.4 mmol) and tetrahydrofuran (15 mL). The solution was cooled to −20° C., and n-butyllithium (nominally 2.5 M in hexane, 3.3 mmol) was added over three minutes. The LDA solution was stirred at −20° C. for 10 minutes, and dimethyl methylphosphonate (136 mg, 1.1 mmol) was added over 1 minute, and the solution was stirred −20° C. for 10 minutes. Diethyl disulfide (147 mg, 1.2 mmol) was added, and the solution was stirred at −20° C. for 15 minutes, and then warmed to 0° C., followed by stirring for 30 minutes. The aldehyde was dissolved in tetrahydrofuran (5 mL) and added over 3 minutes at 0° C., followed by stirring at 0° C. The reaction progress was monitored by TLC, using 2,4-dinitrophenylhydrazine stain to check for the presence of aldehyde. After consumption of the aldehyde, the reaction was warmed to 55° C. for 15 minutes, cooled to room temperature, and then quenched by the addition of saturated ammonium chloride solution. The organic layer was diluted by the addition of hexane (20 mL), and was washed subsequently with 0.5 M sodium hydroxide (2×20 mL), water (2×20 mL), 0.5 M hydrochloric acid (20 mL), and brine (20 mL), and then dried over anhydrous magnesium sulfate. The solvent was removed under vacuum, and the crude product was purified by flash chromatography on silica using a gradient of 100:0 to 95:5 hexane:ethyl acetate as an eluent.

S,S′-diethyl phenylketenedithioacetal (4a) Colorless oil. Yield: 184 mg (82%). ¹H NMR (400 MHz, CDCl₃) δ 7.66-7.58 (m, 1H), 7.38-7.29 (m, 2H), 7.24 (dd, J=7.4, 1.9 Hz, 1H), 7.02 (s, 1H), 2.851 (q, J=7.3 Hz, 2H), 2.850 (q, J=7.3 Hz, 2H), 1.32 (t, J=7.3 Hz, 3H), 1.24 (t, J=7.3 Hz, 3H). ¹³C NMR (101 MHz, CDCl₃) δ 136.6, 135.0, 132.2, 129.5, 128.2, 127.4, 28.1, 27.8, 15.0, 14.4. HRMS (+ESI-QTOF) m/z: [M+H]⁺ calcd for C₁₂H₁₇S₂⁺ 225.0766; found: 225.0766.

S,S′-diethyl (4-fluorophenyl)ketenedithioacetal (4b) Colorless oil. Yield: 205 mg (85%). ¹H NMR (400 MHz, CDCl₃) δ 7.66-7.55 (m, 2H), 7.07-6.98 (m, 2H), 6.98 (s, 1H), 2.85 (q, J=7.3 Hz, 3H), 2.84 (q, J=7.3 Hz, 2H), 1.31 (t, J=7.3 Hz, 3H), 1.23 (t, J=7.4 Hz, 3H). ¹³C NMR (101 MHz, CDCl₃) δ 161.8 (d, J=247.8 Hz), 134.0, 132.7 (d, J=3.5 Hz), 132.0 (d, J=2.0 Hz), 131.2 (d, J=7.9 Hz), 115.1 (d, J=21.4 Hz), 28.1, 27.8, 15.0, 14.4. ¹⁹F NMR (376 MHz, CDCl₃) δ −113.9 (tt, J=8.9, 5.7 Hz). HRMS (+ESI-QTOF) m/z: [M+H]⁺ calcd for C₁₂H₁₆FS₂⁺ 243.0672; found: 243.0668.

S,S′-diethyl (4-chlorophenyl)ketenedithioacetal (4c) Colorless oil. Yield: 206 mg (80%). ¹H NMR (400 MHz, CDCl₃) δ 7.60-7.52 (m, 2H), 7.32-7.27 (m, 2H), 6.93 (s, 1H), 2.847 (q, J=7.3 Hz, 2H), 2.844 (q, J=7.3 Hz, 2H), 1.31 (t, J=7.3 Hz, 3H), 1.23 (t, J=7.3 Hz, 3H). ¹³C NMR (101 MHz, CDCl₃) δ 135.1, 133.4, 133.3, 132.9, 130.7, 128.3, 28.2, 27.8, 15.0, 14.4. HRMS (+ESI-QTOF) m/z: [M+H]⁺ calcd for C₁₂H₁₆ClS₂⁺ 259.0376; found: 259.0377.

S,S′-diisopropyl (4-chlorophenyl)ketenedithioacetal (4d) Eluent: 100:0 to 98:2 hexane:ethyl acetate. Colorless oil. Yield: 122 mg (43%). ¹H NMR (400 MHz, CDCl₃) δ 7.63-7.55 (m, 2H), 7.33-7.25 (m, 2H), 6.99 (s, 1H), 3.53 (hept, J=6.7 Hz, 1H), 3.42 (hept, J=6.7 Hz, 1H), 1.32 (d, J=6.7 Hz, 6H), 1.27 (d, J=6.7 Hz, 6H). ¹³C NMR (101 MHz, CDCl₃) δ 135.5, 135.1, 133.5, 133.0, 130.9, 128.3, 38.2, 37.3, 23.1, 22.7. HRMS (+ESI-QTOF) m/z: [M+H]⁺ calcd for C₁₄H₂₀ClS₂⁺ 287.0689; found: 287.0692.

S,S′-diphenyl (4-chlorophenyl)ketenedithioacetal (4e) Recrystallized from hot acetonitrile to yield white prisms (202 mg, 57%). mp 79-81° C. ¹H NMR (400 MHz, CDCl₃) δ 7.59-7.54 (m, 1H), 7.36-7.29 (m, 6H), 7.29-7.23 (m, 4H), 7.04 (s, 1H). ¹³C NMR (101 MHz, CDCl₃) δ 135.6, 134.4, 133.7, 133.58, 133.55, 132.68, 132.64, 131.1, 130.6, 129.2, 128.9, 128.5, 128.2, 127.4. HRMS (+ESI-QTOF) m/z: [M+H]⁺ calcd for C₂₀H₁₆ClS₂⁺ 355.0376; found: 355.0375.

S,S′-diethyl (4-bromophenyl)ketenedithioacetal (4f) Colorless oil. Yield: 226 mg (75%). ¹H NMR (400 MHz, CDCl₃) δ 7.52-7.47 (m, 2H), 7.46-7.42 (m, 2H), 6.90 (s, 1H), 2.846 (q, J=7.3 Hz, 2H), 2.844 (q, J=7.3 Hz, 2H), 1.31 (t, J=7.3 Hz, 3H), 1.23 (t, J=7.3 Hz, 3H). ¹³C NMR (101 MHz, CDCl₃) δ 135.5, 133.6, 133.2, 131.3, 131.0, 121.1, 28.2, 27.8, 15.0, 14.3. HRMS (+ESI-QTOF) m/z: [M+H]⁺ calcd for C₁₂H₁₆BrS₂⁺ 302.9871; found: 302.9875.

S,S′-diethyl (4-(trifluoromethyl)phenyl)ketenedithioacetal (4g) Colorless oil. Yield: 229 mg (78%). ¹H NMR (400 MHz, CDCl₃) δ 7.74-7.67 (m, 2H), 7.61-7.53 (m, 2H), 6.95 (s, 1H), 2.873 (q, J=7.3 Hz, 2H), 2.867 (q, J=7.3 Hz, 2H), 1.33 (t, J=7.3 Hz, 3H), 1.24 (t, J=7.3 Hz, 3H). ¹³C NMR (101 MHz, CDCl₃) δ 140.1 (q, J_{CF}=1.3 Hz), 135.9, 132.0, 129.5, 128.8 (q, J_{CF}=32.4 Hz), 125.1 (q, J_{CF}=3.8 Hz), 124.4 (q, J_{CF}=270.2 Hz), 28.3, 27.9, 15.0, 14.3. ¹⁹F NMR (377 MHz, CDCl₃) δ −62.5. HRMS (+ESI-QTOF) m/z: [M+H]⁺ calcd for C₁₃H₁₆F₃S₂⁺ 293.0640; found: 293.0637.

S,S′-diethyl (4-nitrophenyl)ketenedithioacetal (4h) Eluent: 100:0 to 85:5 hexane:ethyl acetate. Yellow-red oil that solidifies upon cooling to −20° C. Yield: 158 mg (59%). ¹H NMR (400 MHz, CDCl₃) δ 8.22-8.14 (m, 2H), 7.79-7.72 (m, 2H), 6.89 (s, 1H), 2.90 (q, J=7.3 Hz, 4H), 1.34 (t, J=7.4 Hz, 3H), 1.26 (t, J=7.3 Hz, 3H). ¹³C NMR (101 MHz, CDCl₃) δ 146.1, 143.1, 139.2, 129.8, 129.7, 123.6, 28.6, 28.1, 15.1, 14.1. HRMS (+ESI-QTOF) m/z: [M+H]⁺ calcd for C₁₂H₁₆NO₂S₂⁺ 270.0617; found: 270.0616.

S,S′-diethyl (4-cyanophenyl)ketenedithioacetal (4i) Eluent: 100:0 to 85:5 hexane:ethyl acetate. Yellow oil that solidifies upon cooling to −20° C. Yield: 204 mg (82%). ¹H NMR (600 MHz, CDCl₃) δ 7.73-7.68 (m, 2H), 7.62-7.57 (m, 2H), 6.87 (s, 1H), 2.880 (q, J=7.3 Hz, 2H), 2.877 (q, J=7.3 Hz, 2H), 1.32 (t, J=7.3 Hz, 3H), 1.24 (t, J=7.3 Hz, 3H). ¹³C NMR (151 MHz, CDCl₃) δ 141.0, 137.9, 132.0, 130.7, 129.8, 119.2, 110.1, 28.5, 28.0, 15.0, 14.2. HRMS (+ESI-QTOF) m/z: [M+H]⁺ calcd for C₁₃H₁₆NS₂⁺ 250.0719; found: 250.0716.

S,S′-diethyl (4-tolyl)ketenedithioacetal (4j) Colorless oil. Yield: 206 mg (86%). ¹H NMR (400 MHz, CDCl₃) δ 7.56-7.49 (m, 2H), 7.15 (d, J=8.0 Hz, 2H), 7.01 (s, 1H), 2.848 (q, J=7.3 Hz, 2H), 2.839 (q, J=7.3 Hz, 2H), 2.35 (s, 3H), 1.31 (t, J=7.3 Hz, 3H), 1.23 (t, J=7.3 Hz, 3H). ¹³C NMR (101 MHz, CDCl₃) δ 137.3, 135.5, 133.8, 131.0, 129.5, 128.9, 28.0, 27.8, 21.4, 15.0, 14.4. HRMS (+ESI-QTOF) m/z: [M+H]⁺ calcd for C₁₃H₁₉S₂⁺ 239.0923; found: 239.0928.

S,S′-diethyl (4-methoxyphenyl)ketenedithioacetal (4k) Eluent: 100:0 to 85:5 hexane:ethyl acetate. Colorless oil. Yield: 181 mg (71%). ¹H NMR (400 MHz, CDCl₃) δ 7.65-7.57 (m, 2H), 7.01 (s, 1H), 6.91-6.83 (m, 2H), 3.82 (s, 3H), 2.846 (q, J=7.3 Hz, 2H), 2.833 (q, J=7.3 Hz, 2H), 1.30 (t, J=7.3 Hz, 3H), 1.23 (t, J=7.3 Hz, 3H). ¹³C NMR (101 MHz, CDCl₃) δ 158.9, 135.7, 131.0, 129.4, 129.3, 113.6, 55.4, 28.0, 27.8, 15.0, 14.5. HRMS (+ESI-QTOF) m/z: [M+H]⁺ calcd for C₁₃H₁₉OS₂⁺ 255.0872; found: 255.0875.

S,S′-diethyl (4-(methylthio)phenyl)ketenedithioacetal (4l) Colorless oil. Yield: 184 mg (68%). ¹H NMR (400 MHz, CDCl₃) δ 7.61-7.53 (m, 2H), 7.23-7.14 (m, 2H), 6.96 (s, 1H), 2.849 (q, J=7.3, 2H), 2.835 (q, J=7.3, 2H), 2.49 (s, 3H), 2.84 (q, J=7.3, 2H), 2.49 (s, 3H), 1.30 (t, J=7.3 Hz, 3H), 1.23 (t, J=7.3 Hz, 3H). ¹³C NMR (101 MHz, CDCl₃) δ 137.7, 134.6, 133.4, 131.6, 129.9, 126.0, 28.2, 27.8, 15.8, 15.0, 14.4. HRMS (+ESI-QTOF) m/z: [M+H]⁺ calcd for C₁₃H₁₉S₃⁺ 271.0643; found: 271.0642.

S,S′-dimethyl (4-(difluoromethoxy)phenyl)ketenedithioacetal (4m) Colorless oil. Yield: 141 mg (54%). ¹H NMR (600 MHz, CDCl₃) δ 7.59-7.54 (m, 2H), 7.11-7.03 (m, 2H), 6.73 (s, 1H), 6.51 (t, J=74.0 Hz, 1H), 2.41 (s, 3H), 2.36 (s, 3H). ¹³C NMR (151 MHz, CDCl₃) δ 149.9, 135.9, 133.8, 130.5, 128.5, 119.0, 115.9 (t, J=259.5 Hz), 17.4, 17.2. ¹⁹F{¹H}NMR (565 MHz, CDCl₃) δ −80.7. HRMS (+ESI-QTOF) m/z: [M+H]⁺ calcd for C₁₁H₁₃F₂OS₂⁺: 263.0370; found: 263.0372.

S,S′-dimethyl (4-(trifluoromethoxy)phenyl)ketenedithioacetal (4n) Colorless oil. Yield: 34.2 mg (12%). ¹H NMR (600 MHz, CDCl₃) δ 7.62-7.55 (m, 1H), 7.22-7.13 (m, 1H), 6.71 (s, 1H), 2.41 (s, 3H), 2.36 (s, 3H). ¹³C NMR (151 MHz, CDCl₃) δ 143.5, 136.9, 135.2, 130.5, 128.0, 120.7, 17.5, 17.3. Not observed: —OCF₃. ¹⁹F{¹H} NMR (565 MHz, CDCl₃) δ −57.8.

S,S′-diethyl (4-(trifluoromethylthio)phenyl)ketenedithioacetal (4o) Colorless oil. Yield: 197 mg (61%). ¹H NMR (400 MHz, CDCl₃) δ 7.71-7.62 (m, 2H), 7.63-7.56 (m, 2H), 6.92 (s, 1H), 2.875 (q, J=7.3 Hz, 2H), 2.869 (q, J=7.3 Hz, 2H), 1.32 (t, J=7.3 Hz, 3H), 1.25 (t, J=7.3 Hz, 3H). ¹³C NMR (101 MHz, CDCl₃) δ 139.2, 136.1, 135.8, 132.1, 130.3, 129.7 (q, J_{CF}=308.1 Hz), 122.5 (q, J_{CF}=2.2 Hz), 28.3, 28.0, 15.1, 14.3. ¹⁹F NMR (376 MHz, CDCl₃) δ −42.7. HRMS (+ESI-QTOF) m/z: [M+H]⁺ calcd for C₁₃H₁₆F₃S₃⁺ 325.0361; found: 325.0363.

S,S′-diethyl (4-(dimethylamino)phenyl)ketenedithioacetal (4p) Eluent: 100:0 to 85:5 hexane:ethyl acetate. Pale yellow oil. Yield: 173 mg (65%). ¹H NMR (400 MHz, CDCl₃) δ 7.67-7.58 (m, 2H), 7.02 (s, 1H), 6.72-6.64 (m, 2H), 2.98 (s, 6H), 2.85 (q, J=7.3 Hz, 2H), 2.80 (q, J=7.3 Hz, 2H), 1.28 (t, J=7.3 Hz, 3H), 1.24 (t, J=7.3 Hz, 3H). ¹³C NMR (101 MHz, CDCl₃) δ 149.8, 137.5, 130.9, 125.9, 125.0, 111.7, 40.5, 28.0, 27.8, 15.0, 14.5. HRMS (+ESI-QTOF) m/z: [M+H]⁺ calcd for C₁₄H₂₂NS₂⁺ 268.1188; found: 268.1186.

S,S′-diethyl (3,4-methylenedioxyphenyl)ketenedithioacetal (4q) Eluent: 100:0 to 85:5 hexane:ethyl acetate. Pale yellow oil. Yield: 185 mg (69%). ¹H NMR (400 MHz, CDCl₃) δ 7.40 (d, J=1.7 Hz, 1H), 6.99 (dd, J=8.1, 1.5 Hz, 1H), 6.95 (s, 1H), 6.78 (d, J=8.1 Hz, 1H), 5.96 (s, 2H), 2.85 (q, J=7.3 Hz, 2H), 2.81 (q, J=7.3 Hz, 2H), 1.29 (t, J=7.3 Hz, 3H), 1.24 (t, J=7.3 Hz, 3H). ¹³C NMR (101 MHz, CDCl₃) δ 147.4, 146.9, 135.4, 130.9, 130.1, 124.2, 109.4, 108.1, 101.2, 28.1, 27.8, 15.0, 14.4. HRMS (+ESI-QTOF) m/z: [M+H]⁺ calcd for C₁₃H₁₇O₂S₂⁺ 269.0665; found: 269.0665.

S,S′-diethyl (3,4-(difluoromethylenedioxy)phenyl)ketenedithioacetal (4r) Pale yellow oil. Yield: 216 mg (71%). ¹H NMR (400 MHz, CDCl₃) δ 7.60 (d, J=1.7 Hz, 1H), 7.19 (dd, J=8.4, 1.8 Hz, 1H), 7.00 (d, J=8.3 Hz, 1H), 6.94 (s, 1H), 2.863 (q, J=7.3 Hz, 2H), 2.838 (q, J=7.3 Hz, 2H), 1.30 (t, J=7.3 Hz, 3H), 1.24 (t, J=7.3 Hz, 3H). ¹³C NMR (101 MHz, CDCl₃) δ 143.7, 142.7, 133.3, 133.2, 132.9, 131.8 (t, J_{CF}=250.0 Hz), 125.6, 110.2, 109.0, 28.2, 27.8, 15.1, 14.4. ¹⁹F NMR (376 MHz, CDCl₃) δ −50.1. HRMS (+ESI-QTOF) m/z: [M+H]⁺ calcd for C₁₃H₁₅F₂O₂S₂⁺ 305.0476; found: 305.0477.

S,S′-dimethyl (naphth-1-yl)ketenedithioacetal (4s) Eluent: 100:0 to 90:10 hexane:ethyl acetate. Colorless oil. Yield: 158 mg (59%). Yield: 184 mg (75%). ¹H NMR (400 MHz, CDCl₃) δ 8.01-7.94 (m, 1H), 7.90-7.83 (m, 1H), 7.83-7.76 (m, 1H), 7.57 (dt, J=7.1, 1.1 Hz, 1H), 7.56-7.44 (m, 3H), 7.22 (s, 1H), 2.53 (s, 3H), 2.23 (s, 3H). ¹³C NMR (101 MHz, CDCl₃) δ 137.7, 133.7, 133.5, 131.6, 128.5, 127.8, 127.3, 126.9, 126.0, 125.8, 125.2, 124.5, 17.4, 17.2. HRMS (+ESI-QTOF) m/z: [M+H]⁺ calcd for C₁₄H₁₅S₂⁺ 247.0610; found: 247.0606.

S,S′-diethyl thiophen-2-ylketenedithioacetal (4t) Colorless oil. Yield: 160 mg (60%). ¹H NMR (400 MHz, CDCl₃) δ 7.28-7.24 (m, 1H), 7.13 (dd, J=3.8, 1.2 Hz, 1H), 6.99 (dd, J=5.3, 3.6 Hz, 1H), 2.91 (q, J=7.3 Hz, 3H), 2.82 (q, J=7.3 Hz, 3H), 1.29 (t, J=7.3 Hz, 6H). ¹³C NMR (101 MHz, CDCl₃) δ 140.2, 130.7, 129.8, 128.7, 126.6, 126.1, 28.6, 27.7, 15.0, 14.6. HRMS (+ESI-QTOF) m/z: [M+H]⁺ calcd for C₁₀H₁₅S₃⁺ 231.0330; found: 231.0326.

S,S′-diethyl (1-methylindol-3-yl)ketenedithioacetal (4u) Eluent: 100:0 to 85:5 hexane:ethyl acetate. Colorless oil. Yield: 187 mg (67%). ¹H NMR (400 MHz, CDCl₃) δ 7.99 (s, 1H), 7.72 (dt, J=7.9, 1.0 Hz, 1H), 7.40 (d, J=0.7 Hz, 1H), 7.31 (dt, J=8.2, 1.1 Hz, 1H), 7.26 (ddd, J=8.2, 6.8, 1.1 Hz, 1H), 7.18 (ddd, J=8.0, 6.9, 1.2 Hz, 1H), 3.83 (s, 3H), 2.94 (q, J=7.3 Hz, 2H), 2.82 (q, J=7.3 Hz, 2H), 1.31 (t, J=7.3 Hz, 3H), 1.27 (t, J=7.3 Hz, 3H). ¹³C NMR (101 MHz, CDCl₃) δ 136.2, 130.2, 129.3, 128.1, 124.1, 122.3, 120.0, 118.6, 111.9, 109.4, 33.3, 28.3, 27.8, 15.3, 14.6. HRMS (+ESI-QTOF) m/z: [M+H]⁺ calcd for C₁₅H₂₀NS₂⁺ 278.1032; found: 278.1031.

S,S′-diethyl (2-chloropyridin-5-yl)ketenedithioacetal (4v) Eluent: 100:0 to 90:10 hexane:ethyl acetate. Colorless oil. Yield: 194 mg (75%). ¹H NMR (400 MHz, CDCl₃) δ 8.46 (dd, J=2.5, 0.8 Hz, 1H), 8.05 (dd, J=8.4, 2.5 Hz, 1H), 7.27 (dd, J=8.4, 0.7 Hz, 1H), 6.82 (s, 1H), 2.859 (q, J=7.4, Hz, 2H), 2.853 (q, J=7.4, Hz, 2H), 1.30 (t, J=7.4 Hz, 3H), 1.22 (t, J=7.3 Hz, 3H). ¹³C NMR (101 MHz, CDCl₃) δ 150.3, 149.2, 138.5, 136.9, 131.4, 128.1, 123.5, 28.3, 27.8, 14.9, 14.1. HRMS (+ESI-QTOF) m/z: [M+H]⁺ calcd for C₁₁H₁₆ClNS₂⁺ 263.0326; found: 263.0323.

1,1-bis(ethylthio)-4-phenyl-1,3-butadiene (4w) Eluent: 100:0 to 90:10 hexane:ethyl acetate. Colorless oil. Yield: 143 mg (57%). ¹H NMR (400 MHz, CDCl₃) δ 7.48-7.39 (m, 2H), 7.43 (dd, J=15.7, 10.6 Hz, 1H), 7.36-7.30 (m, 2H), 7.26-7.21 (m, 1H), 6.76 (d, J=10.6 Hz, 1H), 6.59 (d, J=15.7 Hz, 1H), 2.843 (q, J=7.4 Hz, 2H), 2.832 (q, J=7.4 Hz, 2H), 1.29 (t, J=7.5 Hz, 3H), 1.27 (t, J=7.2 Hz, 3H). ¹³C NMR (101 MHz, CDCl₃) δ 137.6, 136.2, 133.1, 132.8, 128.8, 127.8, 126.7, 126.0, 28.2, 27.4, 15.2, 14.4. HRMS (+ESI-QTOF) m/z: [M+H]⁺ calcd for C₁₄H₁₉S₂⁺ 251.0923; found: 251.0919.

1,1-bis(ethylthio)-4,8-dimethylnona-1,7-diene (4x) Eluent: 100:0 to 99:1 hexane:ethyl acetate. Colorless oil. Yield: 185 mg (68%). ¹H NMR (600 MHz, CDCl₃) δ 6.17 (t, J=7.4 Hz, 1H), 5.09 (tp, J=7.1, 1.5 Hz, 1H), 2.76 (q, J=7.3 Hz, 2H), 2.70 (q, J=7.3 Hz, 2H), 2.35 (ddd, J=14.4, 7.1, 5.8 Hz, 1H), 2.23 (dt, J=14.6, 7.6 Hz, 1H), 2.05-1.92 (m, 2H), 1.68 (d, J=1.4 Hz, 3H), 1.60 (d, J=1.3 Hz, 3H), 1.55 (tt, J=14.4, 6.4 Hz, 1H), 1.35 (ddt, J=13.4, 9.5, 6.1 Hz, 1H), 1.22 (dt, J=7.3 Hz, 3H), 1.20 (dt, J=7.3 Hz, 3H), 1.20-1.14 (m, 1H), 0.89 (d, J=6.7 Hz, 3H). ¹³C NMR (151 MHz, CDCl₃) δ 140.1, 131.4, 129.5, 124.9, 38.0, 36.9, 33.2, 27.1, 27.0, 25.9, 25.8, 19.8, 17.8, 15.2, 14.5. HRMS (+ESI-QTOF) m/z: [M+H]⁺ calcd for C₁₅H₂₉S₂⁺ 273.1705; found: 273.1700. Although preferred embodiments have been depicted and described in detail herein, it will be apparent to those skilled in the relevant art that various modifications, additions, substitutions, and the like can be made without departing from the spirit of the application and these are therefore considered to be within the scope of the invention as defined in the claims which follow.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A compound of formula (I) having the following structure: 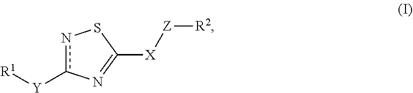
or a stereoisomer, salt, oxide, or solvate thereof, wherein
Y is a bond; —(CH₂)ₙ—; or —CH═;
R¹ is phenyl optionally substituted one or more times with halogen, CF₃, alkoxy, C₁-C₆ alkyl, NO₂, —OCF₃, —OCF₂H, —CN, alkylamine, or alkylthio; pyridine; pyrazine; carboxyalkyl; —CONH₂; alkoxy; alkylsulfide; alkylamine; benzylpiperazine; H; CF₃; and O- and N^{α}-protected amino acids comprising a carboxylate protected by a first protecting group and an amino group protected by a second protecting group; and
X is O or S;
Z is —(CH₂)ₙ—;
R² is selected from the group consisting of C₁-C₆ alkyl optionally substituted one or more times with halogen, alkoxy, or NH with an amine protecting group; C₂-C₁₀ alkenyl; phenyl optionally substituted one or more times with halogen or alkoxy; —COO(CH₂)ₙCH₃; —CO(CH₂)ₙCH₃; and an alcohol; and
n is an integer selected from 0-3,
with the following provisos:
when Y is a bond, R¹ is phenyl, X is S, and n is 0, R² is not methyl, ethyl, or C₃ alkylene;
when Y is a bond, R¹ is phenyl, X is S, and n is 1, R² is not phenyl;
when Y is a bond, R¹ is phenyl, X is O and n is 0, R² is not methyl;
when Y is a bond, R¹ is phenyl substituted with halogen, and n is 0, R² is not methyl;
when Y is a bond, R¹ is alkylsulfide, X is S, and n is 0, R² is not ethyl; and
when Y is a bond, R¹ is methyl, X is S, and n is 0, R² is not ethyl.

2. The compound of claim 1, wherein
Y is a bond and
R¹ is phenyl.

3. The compound of claim 1, wherein
Y is a bond and
R¹ is phenyl optionally substituted one or more times with halogen, CF₃, alkoxy, C₁-C₆ alkyl, NO₂, —OCF₃, —OCF₂H, —CN, alkylamine, or alkylthio.

4. The compound of claim 1, wherein
Y is a bond and
R¹ is pyridine.

5. The compound of claim 1, wherein
Y is a bond and R¹ is pyrazine; carboxyalkyl; —CONH₂; alkoxy; alkylsulfide; alkylamine;
benzylpiperazine; H; CF₃; or O- and N^{α}-protected amino acids comprising a carboxylate protected by a first protecting group and an amino group protected by a second protecting group.

6. The compound of claim 1, wherein
X is S.

7. The compound of claim 6, wherein the compound has a structure selected from

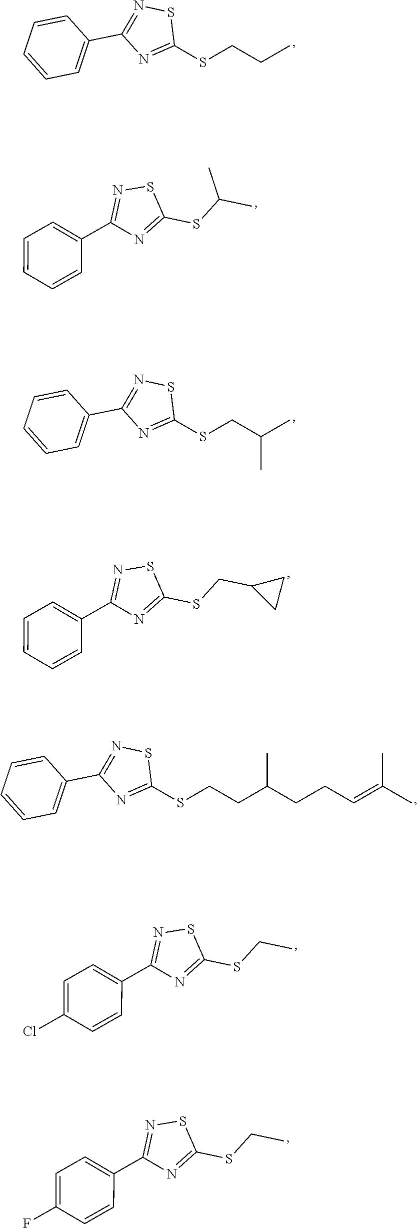

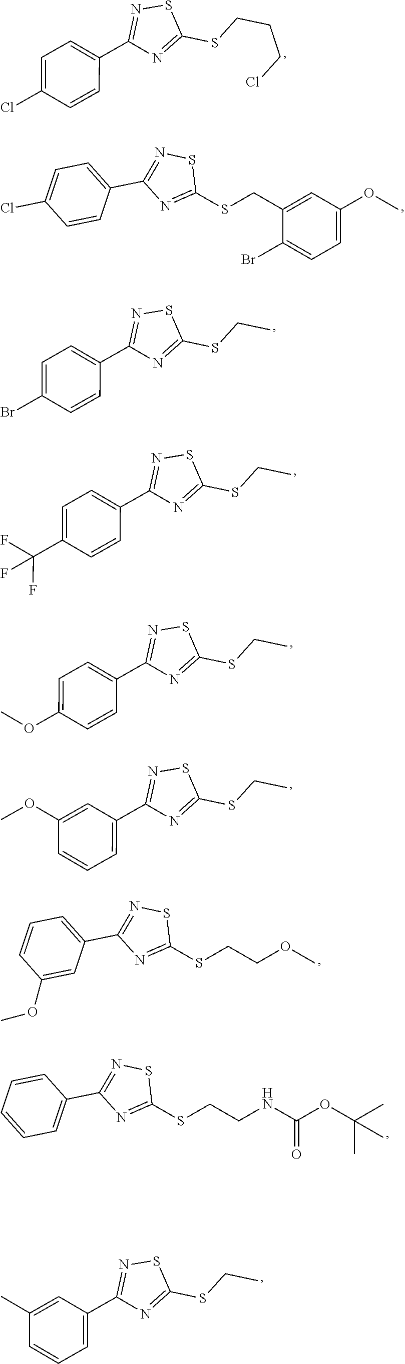

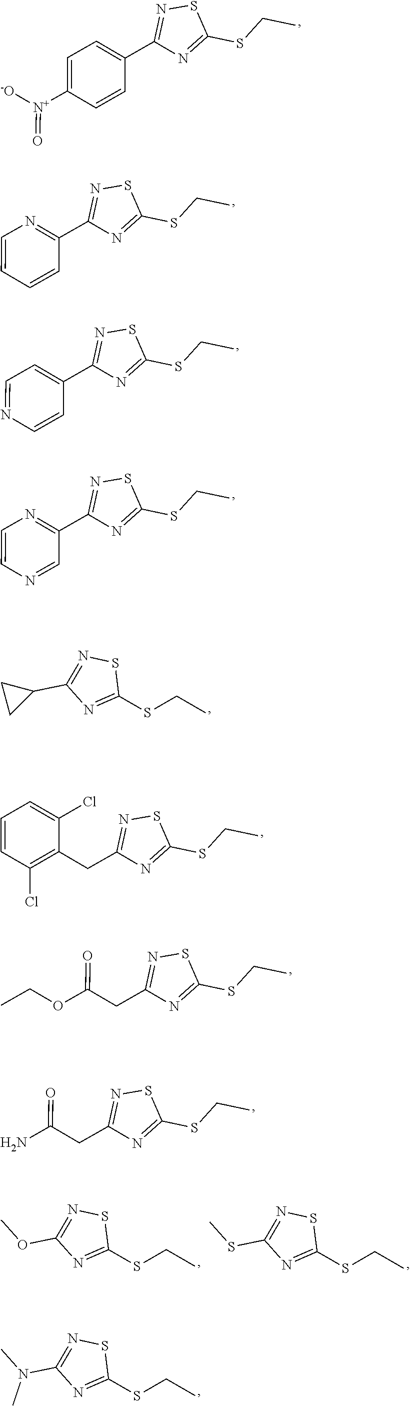

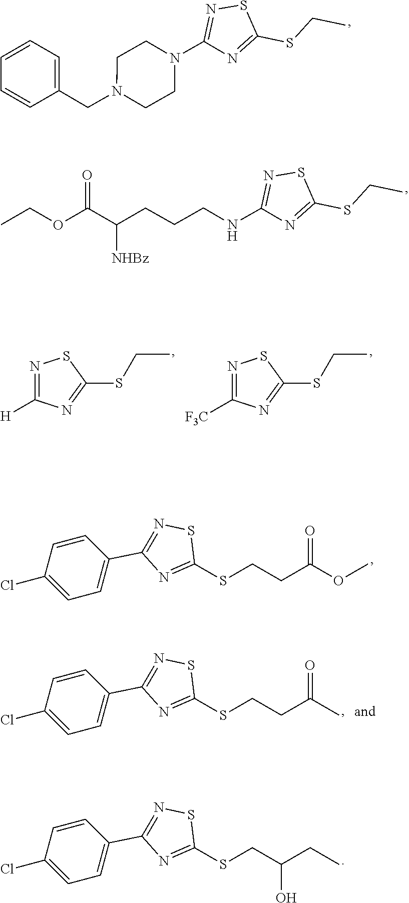

8. The compound of claim 1, wherein
X is O.

9. The compound of claim 7, wherein the compound has a structure selected from

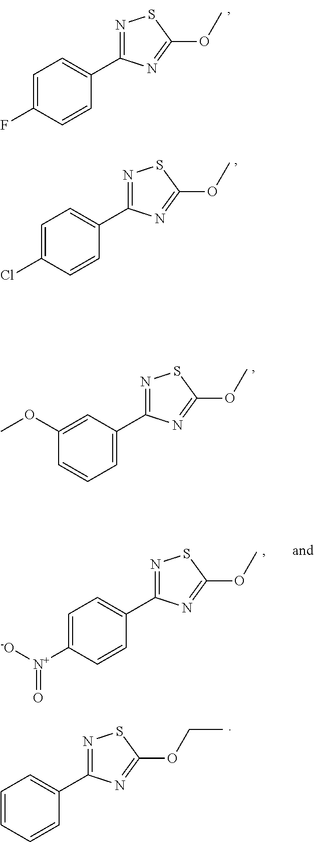

10. The compound of claim 1, wherein
R² is C₁-C₆ alkyl.

11. The compound of claim 1, wherein
R² is C₁-C₆ alkyl substituted with halogen, alkoxy, or NH with an amine protecting group.

12. The compound of claim 1, wherein
R² is C₂-C₁₀ alkenyl.

13. The compound of claim 1, wherein
R² is phenyl.

14. The compound of claim 1, wherein
R² is phenyl substituted one or more times with halogen or alkoxy.

15. The compound of claim 1, wherein
R² is —COO(CH₂)ₙCH₃; —CO(CH₂)ₙCH₃; or an alcohol.

16. A method of treating a plant or a growing media for a nematode, said method comprising:
contacting a plant or a growing media with a compound of formula (I) having the following structure: 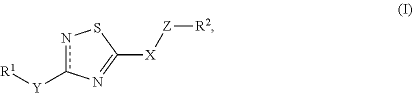
or a stereoisomer, salt, oxide, or solvate thereof, wherein
Y is a bond; —(CH₂)ₙ—; or —CH═;
R¹ is phenyl optionally substituted one or more times with halogen, CF₃, alkoxy, C₁-C₆ alkyl, NO₂, —OCF₃, —OCF₂H, —CN, alkylamine, or alkylthio; pyridine; pyrazine; carboxyalkyl; —CONH₂; alkoxy; alkylsulfide; alkylamine; benzylpiperazine; H; CF₃; and O- and N^{α}-protected amino acids comprising a carboxylate protected by a first protecting group and an amino group protected by a second protecting group; and
X is O or S;
Z is —(CH₂)ₙ—;
R² is selected from the group consisting of C₁-C₆ alkyl optionally substituted one or more times with halogen, alkoxy, or NH with an amine protecting group; C₂-C₁₀ alkenyl; phenyl optionally substituted one or more times with halogen or alkoxy; —COO(CH₂)ₙCH₃; —CO(CH₂)ₙCH₃; and an alcohol; and
n is an integer selected from 0-3,
to treat the plant or growing media for a nematode.

17. The method according to claim 16, wherein said contacting is carried out simultaneously with planting seed in the growing media.

18. The method of claim 16 or claim 17, wherein
Y is a bond and
R¹ is phenyl.

19. The method of claim 16 or claim 17, wherein
Y is a bond and
R¹ is phenyl optionally substituted one or more times with halogen, CF₃, alkoxy, C₁-C₆ alkyl, NO₂, —OCF₃, —OCF₂H, —CN, alkylamine, or alkylthio.

20. The method of claim 16 or claim 17, wherein
Y is a bond and
R¹ is pyridine.

21. The method of claim 16 or claim 17, wherein
Y is a bond and
R¹ is pyrazine; carboxyalkyl; —CONH₂; alkoxy; alkylsulfide; alkylamine;
benzylpiperazine; H; CF₃; and O- and N-protected amino acids comprising a carboxylate protected by a first protecting group and an amino group protected by a second protecting group.

22. The method of claim 16 or claim 17, wherein
X is S.

23. The method of claim 22, wherein the compound has a structure selected from

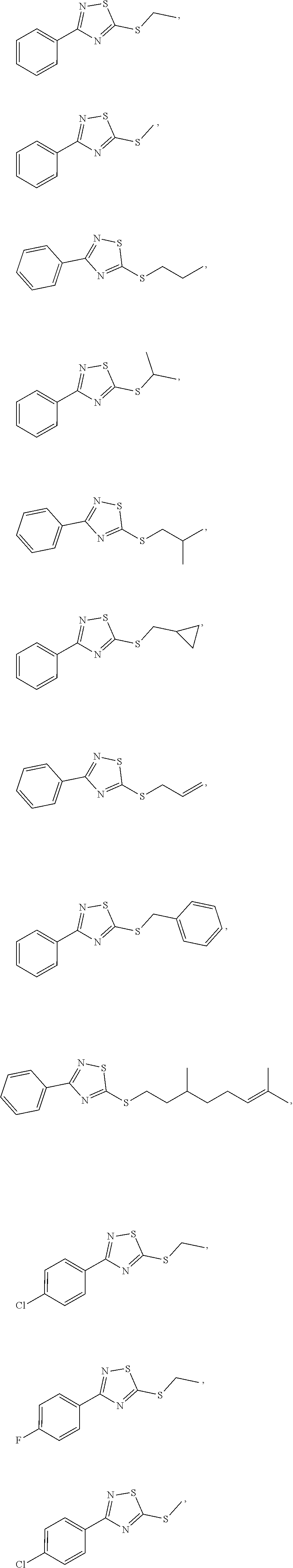

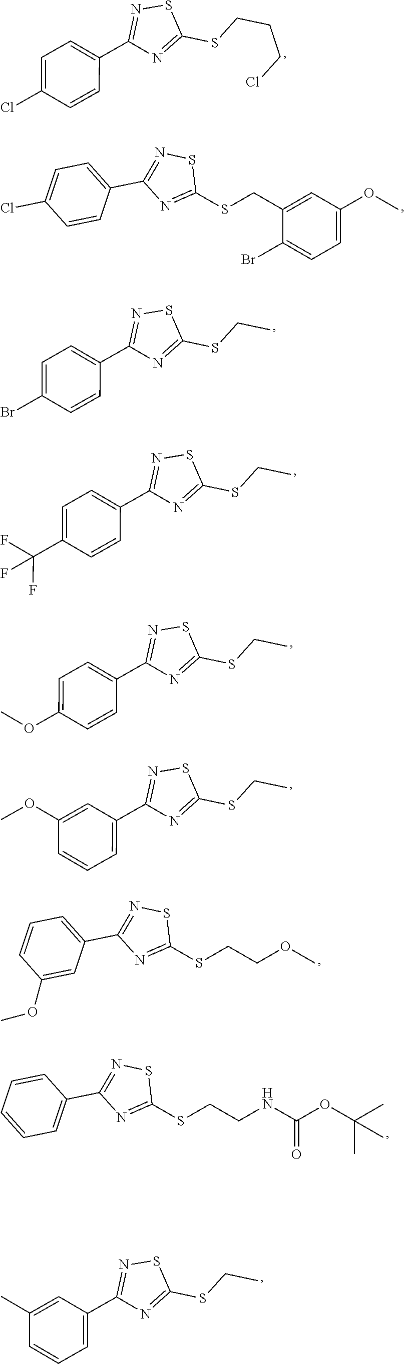

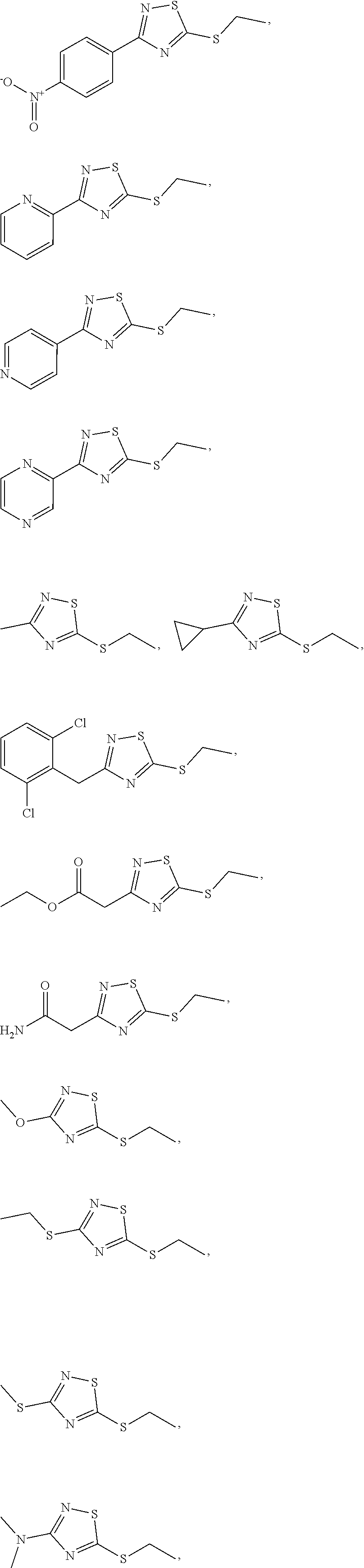

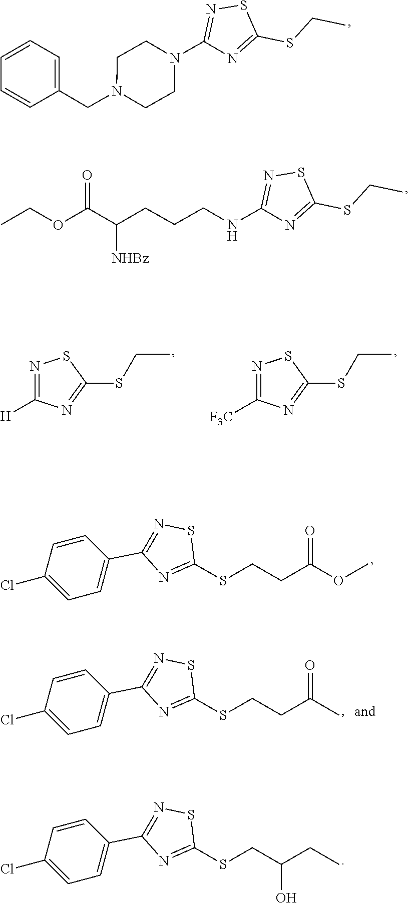

24. The method of claim 16 or claim 17, wherein
X is O.

25. The method of claim 24, wherein the compound has a structure selected from

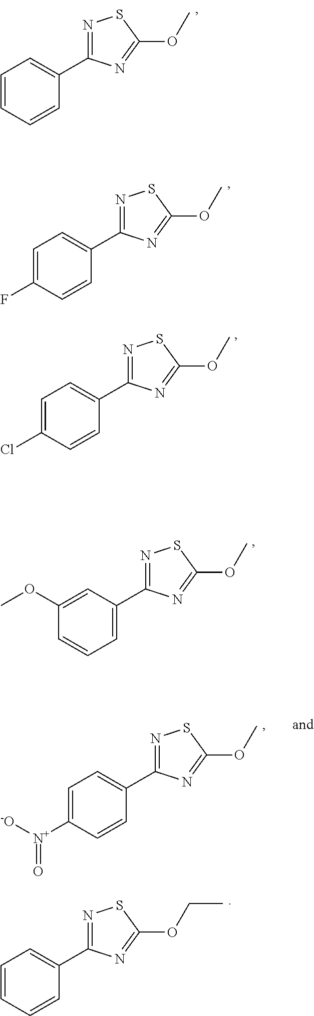

26. The method of claim 16 and claim 17, wherein
R² is C₁-C₆ alkyl.

27. The method of claim 16 or claim 17, wherein
R² is C₁-C₆ alkyl substituted with halogen, alkoxy, or NH with an amine protecting group.

28. The method of claim 16 or claim 17, wherein
R² is C₂-C₁₀ alkenyl.

29. The method of claim 16 or claim 17, wherein
R² is phenyl.

30. The method of claim 16 or claim 17, wherein
R² is phenyl substituted one or more times with halogen or alkoxy.

31. The method of claim 16 or claim 17, wherein
R² is —COO(CH₂)ₙCH₃; —CO(CH₂)ₙCH₃; or an alcohol.

32. A composition comprising:
a compound of formula (I) of any one of claims 1-15 and
an agriculturally acceptable carrier.

33. A compound of formula (II) having the following structure: 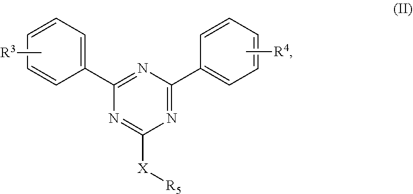
or a stereoisomer, salt, oxide, or solvate thereof, wherein
R³ and R⁴ are independently present or absent, and when present are independently halogen, alkoxy, or C₁-C₆ alkyl;
X is O or S; and
R⁵ is C₁-C₆ alkyl.

34. A method of treating a plant or a growing media for a nematode, said method comprising:
contacting a plant or a growing media with a compound for formula (II) of claim 33 to treat the plant or growing media for a nematode.

35. The method according to claim 34, wherein said contacting is carried out simultaneously with planting seed in the growing media.

36. A composition comprising:
a compound of formula (II) of claim 33 and an agriculturally acceptable carrier.

37. A compound of formula (III) having the following structure: 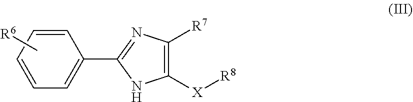
or a stereoisomer, salt, oxide, or solvate thereof, wherein
R⁶ is present or absent and when present is a halogen, alkoxy, or C₁-C₆ alkyl;
R⁷ is selected from H, C₁-C₆ alkyl, alkoxy, and —COO(CH₂)ₙCH₃;
X is S or O;
R⁸ is —(CH₂)ₙCOO(CH₂)nCH₃; and
n is an integer between 0-3.

38. A method of treating a plant or a growing media for a nematode, said method comprising:
contacting a plant or a growing media with a compound for formula (III) of claim 37 to treat the plant or growing media for a nematode.

39. The method according to claim 38, wherein said contacting is carried out simultaneously with planting seed in the growing media.

40. A composition comprising:
a compound of formula (III) of claim 37 and
an agriculturally acceptable carrier.

41. A method of making a compound formula (I) having the following structure: 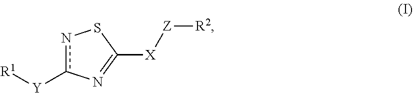
or a stereoisomer, salt, oxide, or solvate thereof, wherein
Y is a bond; —(CH₂)ₙ—; or —CH═;
R¹ is phenyl optionally substituted one or more times with halogen, CF₃, alkoxy, C₁-C₆ alkyl, NO₂, —OCF₃, —OCF₂H, —CN, alkylamine, or alkylthio; pyridine; pyrazine; carboxyalkyl; —CONH₂; alkoxy; alkylsulfide; alkylamine; benzylpiperazine; H; CF₃; and O- and N^{α}-protected amino acids comprising a carboxylate protected by a first protecting group and an amino group protected by a second protecting group; and
X is O or S;
Z is —(CH₂)ₙ—;
R² is selected from the group consisting of C₁-C₆ alkyl optionally substituted one or more times with halogen, alkoxy, or NH with an amine protecting group; C₂-C₁₀ alkenyl; phenyl optionally substituted one or more times with halogen or alkoxy; —COO(CH₂)ₙCH₃; —CO(CH₂)ₙCH₃; and an alcohol; and
n is an integer selected from 0-3,
said method comprising:
providing a starting material comprising an amidine, isourea, guanidine, or isothiourea molecule;
reacting the starting material with carbon disulfide or an alkyl imidazole-1-carbodithioate molecule to form a dithiocarbamate compound; and
converting the dithiocarbamate compound to a compound of formula (I).

42. The method according to claim 41, wherein said reacting is carried out in the presence of a base.

43. The method according to claim 41 or claim 42, wherein the base is selected from the group consisting of DBU, DBN, potassium carbonate, potassium phosphate, sodium bicarbonate, and mixtures thereof.

44. The method according to any one of claims 41-43, wherein said converting is carried out in the presence of an oxidizing agent.

45. A compound of formula (IV) having the following structure: 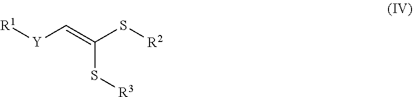
or a stereoisomer, salt, oxide, or solvate thereof, wherein
Y is a bond or C₂-C₁₀ alkylene;
R¹ is optional, and when present is phenyl optionally substituted at one or more positions with halogen, CF₃, NO₂, —CN, C₁-C₆ alkyl, C₁-C₆ alkoxy optionally substituted one or more times with halogen, C₁-C₆ alkylthio, —SCF₃, C₁-C₆ alkylamine; benzodioxolyl optionally substituted one or more times with halogen; naphthalene; thiophene; indole optionally substituted one or more times with C₁-C₆ alkyl; and pyridine optionally substituted one or more times with halogen; and
R² and R³ are independently selected from C₁-C₈ alkyl and phenyl,
with the following provisos:
when Y is a bond and R¹ is phenyl or phenyl substituted one time with a halogen, NO₂ or MeO, R² and R³ are not ethyl;
when Y is a bond and R¹ is naphthalene, R² and R³ are not methyl; and
when Y is C₂ alkylene and R¹ is phenyl, R² and R³ are not ethyl.

46. The compound according to claim 45, wherein Y is bond.

47. The compound according to claim 45 or claim 46, wherein R¹ is phenyl.

48. The compound according to claim 45 or claim 46, wherein R¹ is phenyl substituted with halogen.

49. The compound according to claim 45 or claim 46, wherein R¹ is phenyl substituted with CF₃.

50. The compound according to claim 45 or claim 46, wherein R¹ is phenyl substituted with NO₂.

51. The compound according to claim 45 or claim 46, wherein R¹ is phenyl substituted with —CN.

52. The compound according to claim 45 or claim 46, wherein R¹ is phenyl substituted with C₁-C₆ alkyl.

53. The compound according to claim 45 or claim 46, wherein R¹ is phenyl substituted with C₁-C₆ alkoxy optionally substituted one or more times with halogen.

54. The compound according to claim 53, wherein R¹ is phenyl substituted with —OCF₃ or —OCF₂H.

55. The compound according to claim 45 or claim 46, wherein R¹ is phenyl substituted with C₁-C₆ alkylthio.

56. The compound according to claim 55, wherein R¹ is phenyl substituted with —SCF₃.

57. The compound according to claim 45 or claim 46, wherein R¹ is phenyl substituted with C₁-C₆ alkylamine.

58. The compound according to claim 45 or claim 46, wherein R¹ is benzodioxolyl optionally substituted one or more times with halogen.

59. The compound according to claim 45 or claim 46, wherein R¹ is thiophene.

60. The compound according to claim 45 or claim 46, wherein R¹ is indole optionally substituted one or more times with C₁-C₆ alkyl.

61. The compound according to claim 45 or claim 46, wherein R¹ is pyridine optionally substituted one or more times with halogen.

62. The compound according to claim 45 having the following structure:

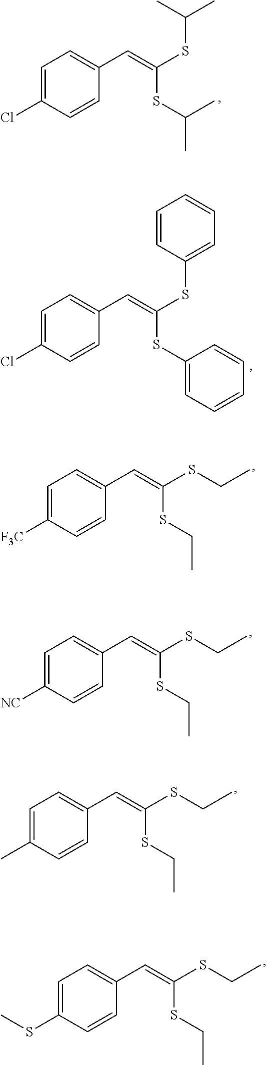

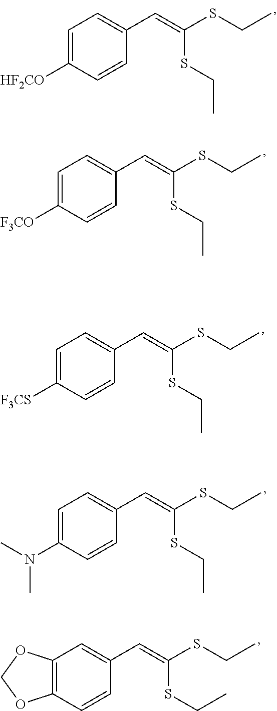

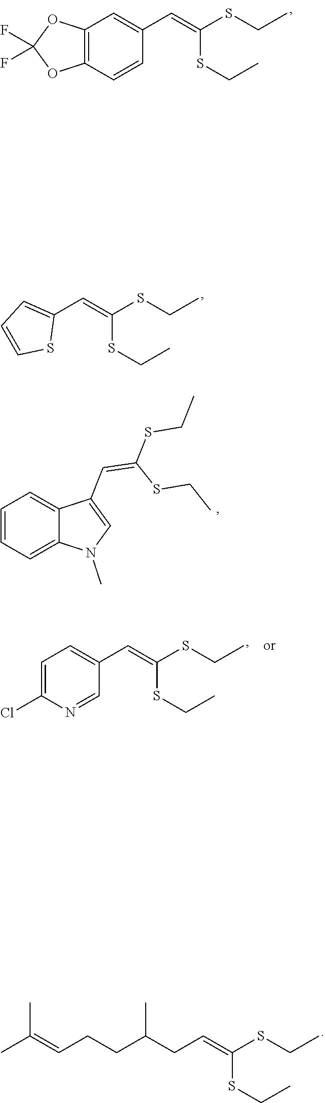

63. A method of treating a plant or growing media for a nematode, said method comprising:
contacting a plant or growing media with a compound of formula (IV) having the following structure: 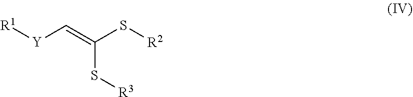
or a stereoisomer, salt, oxide, or solvate thereof, wherein
Y is a bond or C₂-C₁₀ alkylene;
R¹ is optional, and when present is phenyl optionally substituted at one or more positions with halogen, CF₃, NO₂, —CN, C₁-C₆ alkyl, C₁-C₆ alkoxy optionally substituted one or more times with halogen, C₁-C₆ alkylthio, —SCF₃, C₁-C₆ alkylamine; benzodioxolyl optionally substituted one or more times with halogen; naphthalene; thiophene; indole optionally substituted one or more times with C₁-C₆ alkyl; and pyridine optionally substituted one or more times with halogen; and
R² and R³ are independently selected from C₁-C₈ alkyl and phenyl to treat the plant or growing media for a nematode.

64. The method according to claim 63, wherein said contacting is carried out simultaneously with planting seed in the growing media.

65. The method according to claim 63 or claim 64, wherein Y is bond.

66. The method according to claim 63 or claim 64, wherein R¹ is phenyl.

67. The method according to claim 63 or claim 64, wherein R¹ is phenyl substituted with halogen.

68. The method according to claim 63 or claim 64, wherein R¹ is phenyl substituted with CF₃.

69. The method according to claim 63 or claim 64, wherein R¹ is phenyl substituted with NO₂.

70. The method according to claim 63 or claim 64, wherein R¹ is phenyl substituted with —CN.

71. The method according to claim 63 or claim 64, wherein R¹ is phenyl substituted with C₁-C₆ alkyl.

72. The method according to claim 63 or claim 64, wherein R¹ is phenyl substituted with C₁-C₆ alkoxy optionally substituted one or more times with halogen.

73. The method according to claim 72, wherein R¹ is phenyl substituted with —OCF₃ or —OCF₂H.

74. The method according to claim 63 or claim 64, wherein R¹ is phenyl substituted with C₁-C₆ alkylthio.

75. The method according to claim 63 or claim 64, wherein R¹ is phenyl substituted with —SCF₃.

76. The method according to claim 63 or claim 64, wherein R¹ is phenyl substituted with C₁-C₆ alkylamine.

77. The method according to claim 63 or claim 64, wherein R¹ is benzodioxolyl optionally substituted one or more times with halogen.

78. The method according to claim 63 or claim 64, wherein R¹ is naphthalene.

79. The method according to claim 63 or claim 64, wherein R¹ is thiophene.

80. The method according to claim 63 or claim 64, wherein R¹ is indole optionally substituted one or more times with C₁-C₆ alkyl.

81. The method according to claim 63 or claim 64, wherein R¹ is pyridine optionally substituted one or more times with halogen.

82. The method according to claim 63 or claim 64 having the following structure:

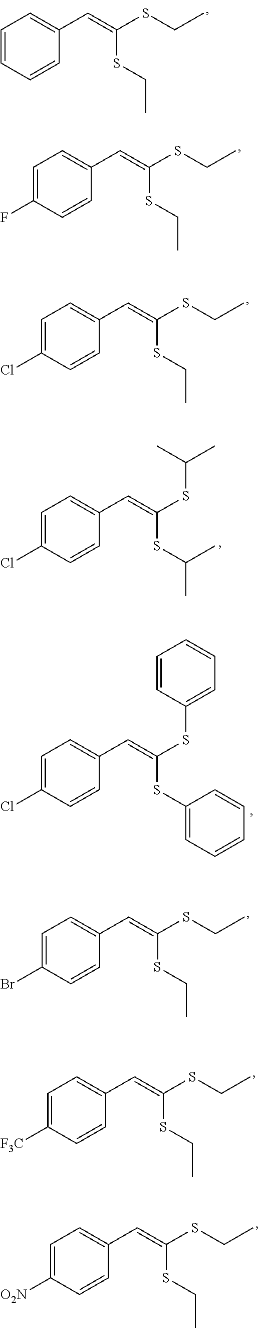

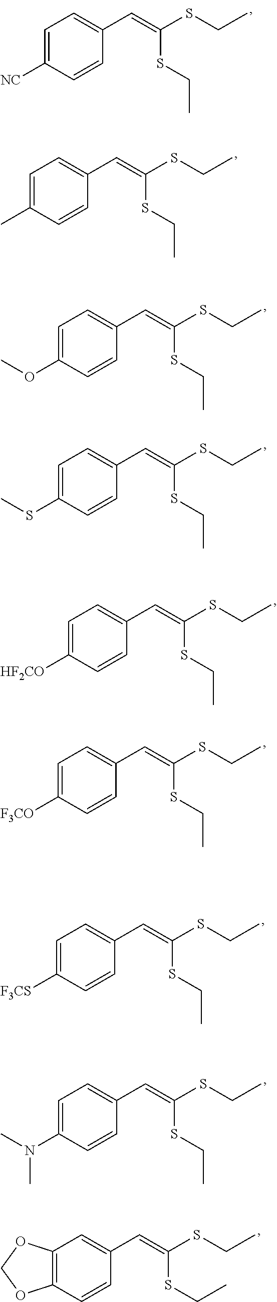

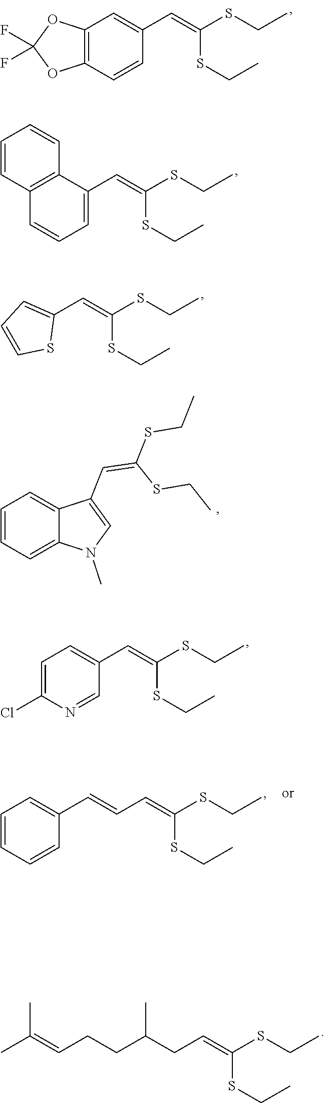

83. A method of treating a plant or growing media for a nematode, said method comprising:
contacting a plant or growing media with a compound of formula (V) having the following structure: 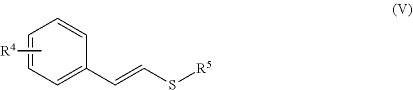
or a stereoisomer, salt, oxide, or solvate thereof, wherein
R⁴ is selected from C₁-C₆ alkyl, alkoxy, CF₃, and halogen and
R⁵ is C₁-C₆ alkyl to treat the plant or growing media for a nematode.

84. The method according to claim 83, wherein said contacting is carried out simultaneously with planting seed in the growing media.

85. The method according to claim 83, wherein the compound of formula (V) has the following structure:

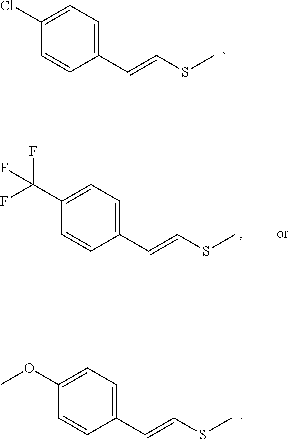

86. A method of forming a ketene dithioacetal compound having a structure of formula (VI) 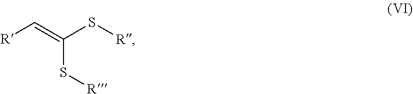
said method comprising:
providing a dimethyl methyl phosphonate compound having a structure of 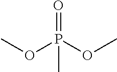
and
reacting the dimethyl methyl phosphonate compound with a disulfide compound and an aldehyde to produce the compound of formula (VI), wherein R′, R″, and R′″ are any compatible substituent.

87. The method according to claim 86, wherein said method is carried out in a single reaction container.

88. The method according to claim 86 or 87, wherein said reacting comprises:
combining the dimethyl methyl phosphonate with the disulfide in the presence of an amide base.

89. The method according to claim 88, wherein said amide base is selected from the group consisting of lithium diisopropylamide (LDA), lithium tetramethylpiperidine (LiTMP), and sodium or potassium analogs.

90. The method according to any one of claims 86-89, wherein the disulfide compound is selected from ethyl disulfide, dimethyl disulfide, diisopropyl disulfide, and diphenyl disulfide.

91. The method according to any one of claims 86-90, wherein said reacting the dimethyl methyl phosphonate compound with a disulfide compound produces a compound having a structure of formula (VII), as follows: 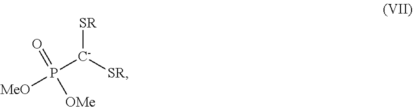
wherein R is any compatible substituent.

92. The method according to any one of claims 86-91, wherein the compound of formula (VII) is reacted with an aldehyde to produce the compound of formula (VI).

93. A composition comprising:
a compound of formula (IV) of any one of claims 45-62 and
an agriculturally acceptable carrier.
